SEQUENCE LISTING (100 sequences)

SEQ ID NO: 1 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 2 - DNA, 2511 bp, Homo sapiens
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgttttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggttcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggttcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatacaactaaaattataaccaaagttgtggaaccaaaaattaaagtgattgaaggcagtcttcagcctattatcaaaactgaaggacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggttctagaagacgattaagggaaggtcgttctcagtga

SEQ ID NO: 3 - protein, 779 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 4 - DNA, 2340 bp, Homo sapiens
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgtgttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggatcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggatcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctataagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggatctagaagacgattaagggaaggtcgttctcagtga

SEQ ID NO: 5 - protein, 809 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 6 - DNA, 2430 bp, Homo sapiens
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgtgttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggatcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggatcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatagacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggatctagaagacgattaagggaaggtcgttctcagtga

SEQ ID NO: 7 - protein, 808 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEDTPVRKLQANKKVQGSRRRLREGRSQ

SEQ ID NO: 8 - DNA, 2427 bp, Homo sapiens
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgtgttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggatcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggatcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatacaactaaaattataaccaaagttgtggaaccaaaaattaaagtgattgaaggcagtcttcagcctattatcaaaactgaaggacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagacacacccgtgaggaagttgcaagccaacaaaaaagttcaaggatctagaagacgattaagggaaggtcgttctcagtga

SEQ ID NO: 9 - protein, 721 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYGPEIKYTRISTGGGETEETLKKLLQEDTPVRKLQANKKVQGSRRRLREGRSQ

SEQ ID NO: 10 - DNA, 2166 bp, Homo sapiens
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgttttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggttcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggttcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatggtcctgaaataaaatacactaggatttctactggaggtggagaaacagaagaaactctgaagaaattgttacaagaagacacacccgtgaggaagttgcaagccaacaaaaaagttcaaggttctagaagacgattaagggaaggtcgttctcagtga

SEQ ID NO: 11 - protein, 752 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEEDTPVRKLQANKKVQGSRRRLREGRSQ

SEQ ID NO: 12 - DNA, 2259 bp, Homo sapiens
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgttttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggttcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggttcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctataaaccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggacacacccgtgaggaagttgcaagccaacaaaaaagttcaaggttctagaagacgattaagggaaggtcgttctcagtga

SEQ ID NO: 13 - protein, 749 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYGPEIKYTRISTGGGETEETLKKLLQEEVTKVTKFIEGGDGHLFEDEEIKRLLQGDTPVRKLQANKKVQGSRRRLREGRSQ

SEQ ID NO: 14 - DNA, 2250 bp, Homo sapiens
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgttttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggttcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggttcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggttctagaagacgattaagggaaggtcgttctcagtga

SEQ ID NO: 15 - protein, 809 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 16 - DNA, 2430 bp, Homo sapiens
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgttttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggttcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggttcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatagacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggttctagaagacgattaagggaaggtcgttctcagtga

SEQ ID NO: 17 - protein, 346 aa, Homo sapiens
MDTGPDQSYFSGNHWFVFSVYLLTFLVGLPLNLLALVVFVGKLRCRPVAVDVLLLNLTASDLLLLLFLPFRMVEAANGMHWPLPFILCPLSGFIFFTTIYLTALFLAAVSIERFLSVAHPLWYKTRPRLGQAGLVSVACWLLASAHCSVVYVIEFSGDISHSQGTNGTCYLEFWKDQLAILLPVRLEMAVVLFVVPLIITSYCYSRLVWILGRGGSHRRQRRVAGLVAATLLNFLVCFGPYNVSHVVGYICGESPVWRIYVTLLSTLNSCVDPFVYYFSSSGFQADFHELLRRLCGLWGQWQQESSMELKEQKGGEEQRADRPAERKTSEHSQGCGTGGQVACAEN

SEQ ID NO: 18 - DNA, 1041 bp, Homo sapiens
atggatacaggccccgaccagtcctacttctccggcaatcactggttcgtcttctcggtgtaccttctcactttcctggtggggctccccctcaacctgctggccctggtggtcttcgtgggcaagctgcggtgccgcccggtggccgtggacgtgctcctgctcaacctgaccgcctcggacctgctcctgctgctgttcctgcctttccgcatggtggaggcagccaatggcatgcactggcccctgcccttcatcctctgcccactctctggattcatcttcttcaccaccatctatctcaccgccctcttcctggcagctgtgagcattgaacgcttcctgagtgtggcccacccactgtggtacaagacccggccgaggctggggcaggcaggtctggtgagtgtggcctgctggctgttggcctctgctcactgcagcgtggtctacgtcatagaattctcaggggacatctcccacagccagggcaccaatgggacctgctacctggagttctggaaggaccagctagccatcctcctgcccgtgcggctggagatggctgtggtcctctttgtggtcccgctgatcatcaccagctactgctacagccgcctggtgtggatcctcggcagagggggcagccaccgccggcagaggagggtggcggggctggtggcggccacgctgctcaacttccttgtctgctttgggccctacaacgtgtcccatgtcgtgggctatatctgcggtgaaagcccggtgtggaggatctacgtgacgcttctcagcaccctgaactcctgtgtcgacccctttgtctactacttctcctcctccgggttccaagccgactttcatgagctgctgaggaggttgtgtgggctctggggccagtggcagcaggagagcagcatggagctgaaggagcagaagggaggggaggagcagagagcggaccgaccagctgaaagaaagaccagtgaacactcacagggctgtggaactggtggccaggtggcctgtgctgaaaactag

SEQ ID NO: 19 - protein, 346 aa, Homo sapiens
MDTGPDQSYFSGNHWFVFSVYLLTFLVGLPLNLLALVVFVGKLQRRPVAVDVLLLNLTASDLLLLLFLPFRMVEAANGMHWPLPFILCPLSGFIFFTTIYLTALFLAAVSIERFLSVAHPLWYKTRPRLGQAGLVSVACWLLASAHCSVVYVIEFSGDISHSQGTNGTCYLEFRKDQLAILLPVRLEMAVVLFVVPLIITSYCYSRLVWILGRGGSHRRQRRVAGLLAATLLNFLVCFGPYNVSHVVGYICGESPAWRIYVTLLSTLNSCVDPFVYYFSSSGFQADFHELLRRLCGLWGQWQQESSMELKEQKGGEEQRADRPAERKTSEHSQGCGTGGQVACAES

SEQ ID NO: 20 - DNA, 1041 bp, Homo sapiens
atggatacaggccccgaccagtcctacttctccggcaatcactggttcgtcttctcggtgtaccttctcactttcctggtggggctccccctcaacctgctggccctggtggtcttcgtgggcaagctgcagcgccgcccggtggccgtggacgtgctcctgctcaacctgaccgcctcggacctgctcctgctgctgttcctgcctttccgcatggtggaggcagccaatggcatgcactggcccctgcccttcatcctctgcccactctctggattcatcttcttcaccaccatctatctcaccgccctcttcctggcagctgtgagcattgaacgcttcctgagtgtggcccacccactgtggtacaagacccggccgaggctggggcaggcaggtctggtgagtgtggcctgctggctgttggcctctgctcactgcagcgtggtctacgtcatagaattctcaggggacatctcccacagccagggcaccaatgggacctgctacctggagttccggaaggaccagctagccatcctcctgcccgtgcggctggagatggctgtggtcctctttgtggtcccgctgatcatcaccagctactgctacagccgcctggtgtggatcctcggcagagggggcagccaccgccggcagaggagggtggcggggctgttggcggccacgctgctcaacttccttgtctgctttgggccctacaacgtgtcccatgtcgtgggctatatctgcggtgaaagcccggcatggaggatctacgtgacgcttctcagcaccctgaactcctgtgtcgacccctttgtctactacttctcctcctccgggttccaagccgactttcatgagctgctgaggaggttgtgtgggctctggggccagtggcagcaggagagcagcatggagctgaaggagcagaagggaggggaggagcagagagcggaccgaccagctgaaagaaagaccagtgaacactcacagggctgtggaactggtggccaggtggcctgtgctgaaagctag

SEQ ID NO: 21 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTAFRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 22 - DNA, 1479 bp, Homo sapiens
atggagcccagcagcaagaagctgacgggtcgcctcatgctggctgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcctttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgataagacacccgaggagctgttccatcccctgggggctgattcccaagtgtga

SEQ ID NO: 23 - protein, 3396 aa, Homo sapiens
MFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEATTIDLSILAETASPSLSKEPQMVSDRTTPIIPLVDELPVIPTEFPPVGNIVSFEQKATVQPQAITDSLATKLPTPTGSTKKPWDMDDYSPSASGPLGKLDISEIKEEVLQSTTGVSHYATDSWDGVVEDKQTQESVTQIEQIEVGPLVTSMEILKHIPSKEFPVTETPLVTARMILESKTEKKMVSTVSELVTTGHYGFTLGEEDDEDRTLTVGSDESTLIFDQIPEVITVSKTSEDTIHTHLEDLESVSASTTVSPLIMPDNNGSSMDDWEERQTSGRITEEFLGKYLSTTPFPSQHRTEIELFPYSGDKILVEGISTVIYPSLQTEMTHRRERTETLIPEMRTDTYTDEIQEEITKSPFMGKTEEEVFSGMKLSTSLSEPIHVTESSVEMTKSFDFPTLITKLSAEPTEVRDMEEDFTATPGTTKYDENITTVLLAHGTLSVEAATVSKWSWDEDNTTSKPLESTEPSASSKLPPALLTTVGMNGKDKDIPSFTEDGADEFTLIPDSTQKQLEEVTDEDIAAHGKFTIRFQPTTSTGIAEKSTLRDSTTEEKVPPITSTEGQVYATMEGSALGEVEDVDLSKPVSTVPQFAHTSEVEGLAFVSYSSTQEPTTYVDSSHTIPLSVIPKTDWGVLVPSVPSEDEVLGEPSQDILVIDQTRLEATISPETMRTTKITEGTTQEEFPWKEQTAEKPVPALSSTAWTPKEAVTPLDEQEGDGSAYTVSEDELLTGSERVPVLETTPVGKIDHSVSYPPGAVTEHKVKTDEVVTLTPRIGPKVSLSPGPEQKYETEGSSTTGFTSSLSPFSTHITQLMEETTTEKTSLEDIDLGSGLFEKPKATELIEFSTIKVTVPSDITTAFSSVDRLHTTSAFKPSSAITKKPPLIDREPGEETTSDMVIIGESTSHVPPTTLEDIVAKETETDIDREYFTTSSPPATQPTRPPTVEDKEAFGPQALSTPQPPASTKFHPDINVYIIEVRENKTGRMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVSVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPGPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 24 - DNA, 7230 bp, Homo sapiens
atgttcataaatataaagagcatcttatggatgtgttcaaccttaatagtaacccatgcgctacataaagtcaaagtgggaaaaagcccaccggtgaggggctccctctctggaaaagtcagcctaccttgtcatttttcaacgatgcctactttgccacccagttacaacaccagtgaatttctccgcatcaaatggtctaagattgaagtggacaaaaatggaaaagatttgaaagagactactgtccttgtggcccaaaatggaaatatcaagattggtcaggactacaaagggagagtgtctgtgcccacacatcccgaggctgtgggcgatgcctccctcactgtggtcaagctgctggcaagtgatgcgggtctttaccgctgtgacgtcatgtacgggattgaagacacacaagacacggtgtcactgactgtggatggggttgtgtttcactacagggcggcaaccagcaggtacacactgaattttgaggctgctcagaaggcttgtttggacgttggggcagtcatagcaactccagagcagctctttgctgcctatgaagatggatttgagcagtgtgacgcaggctggctggctgatcagactgtcagatatcccatccgggctcccagagtaggctgttatggagataagatgggaaaggcaggagtcaggacttatggattccgttctccccaggaaacttacgatgtgtattgttatgtggatcatctggatggtgatgtgttccacctcactgtccccagtaaattcaccttcgaggaggctgcaaaagagtgtgaaaaccaggatgccaggctggcaacagtgggggaactccaggcggcatggaggaacggctttgaccagtgcgattacgggtggctgtcggatgccagcgtgcgccaccctgtgactgtggccagggcccagtgtggaggtggtctacttggggtgagaaccctgtatcgttttgagaaccagacaggcttccctccccctgatagcagatttgatgcctactgctttaaacgtcgaatgagtgatttgagtgtaattggtcatccaatagattcagaatctaaagaagatgaaccttgtagtgaagaaacagatccagtgcatgatctaatggctgaaattttacctgaattccctgacataattgaaatagacctataccacagtgaagaaaatgaagaagaagaagaagagtgtgcaaatgctactgatgtgacaaccaccccatctgtgcagtacataaatgggaagcatctcgttaccactgtgcccaaggacccagaagctgcagaagctaggcgtggccagtttgaaagtgttgcaccttctcagaatttctcggacagctctgaaagtgatactcatccatttgtaatagccaaaacggaattgtctactgctgtgcaacctaatgaatctacagaaacaactgagtctcttgaagttacatggaagcctgagacttaccctgaaacatcagaacatttttcaggtggtgagcctgatgttttccccacagtcccattccatgaggaatttgaaagtggaacagccaaaaaaggggcagaatcagtcacagagagagatactgaagttggtcatcaggcacatgaacatactgaacctgtatctctgtttcctgaagagtcttcaggagagattgccattgaccaagaatctcagaaaatagcctttgcaagggctacagaagtaacatttggtgaagaggtagaaaaaagtacttctgtcacatacactcccactatagttccaagttctgcatcagcatatgtttcagaggaagaagcagttaccctaataggaaatccttggccagatgacctgttgtctaccaaagaaagctgggtagaagcaactcctagacaagttgtagagctctcagggagttcttcgattccaattacagaaggctctggagaagcagaagaagatgaagatacaatgttcaccatggtaactgatttatcacagagaaatactactgatacactcattactttagacactagcaggataatcacagaaagcttttttgaggttcctgcaaccaccatttatccagtttctgaacaaccttctgcaaaagtggtgcctaccaagtttgtaagtgaaacagacacttctgagtggatttccagtaccactgttgaggaaaagaaaaggaaggaggaggagggaactacaggtacggcttctacatttgaggtatattcatctacacagagatcggatcaattaattttaccctttgaattagaaagtccaaatgtagctacatctagtgattcaggtaccaggaaaagttttatgtccttgacaacaccaacacagtctgaaagggaaatgacagattctactcctgtctttacagaaacaaatacattagaaaatttgggggcacagaccactgagcacagcagtatccatcaacctggggttcaggaagggctgaccactctcccacgtagtcctgcctctgtctttatggagcagggctctggagaagctgctgccgacccagaaaccaccactgtttcttcattttcattaaacgtagagtatgcaattcaagccgaaaaggaagtagctggcactttgtctccgcatgtggaaactacattctccactgagccaacaggactggttttgagtacagtaatggacagagtagttgctgaaaatataacccaaacatccagggaaatagtgatttcagagcgattaggagaaccaaattatggggcagaaataaggggcttttccacaggttttcctttggaggaagatttcagtggtgactttagagaatactcaacagtgtctcatcccatagcaaaagaagaaacggtaatgatggaaggctctggagatgcagcatttagggacacccagacttcaccatctacagtacctacttcagttcacatcagtcacatatctgactcagaaggacccagtagcaccatggtcagcacttcagccttcccctgggaagagtttacatcctcagctgagggctcaggtgagcaactggtcacagtcagcagctctgttgttccagtgcttcccagtgctgtgcaaaagttttctggtacagcttcctccattatcgacgaaggattgggagaagtgggtactgtcaatgaaattgatagaagatccaccattttaccaacagcagaagtggaaggtacgaaagctccagtagagaaggaggaagtaaaggtcagtggcacagtttcaacaaactttccccaaactatagagccagccaaattatggtctaggcaagaagtcaaccctgtaagacaagaaattgaaagtgaaacaacatcagaggaacaaattcaagaagaaaagtcatttgaatcccctcaaaactctcctgcaacagaacaaacaatctttgattcacagacatttactgaaactgaactcaaaaccacagattattctgtactaacaacaaagaaaacttacagtgatgataaagaaatgaaggaggaagacacttctttagttaacatgtctactccagatccagatgcaaatggcttggaatcttacacaactctccctgaagctactgaaaagtcacattttttcttagctactgcattagtaactgaatctataccagctgaacatgtagtcacagattcaccaatcaaaaaggaagaaagtacaaaacattttccgaaaggcatgagaccaacaattcaagagtcagatactgagctcttattctctggactgggatcaggagaagaagttttacctactctaccaacagagtcagtgaattttactgaagtggaacaaatcaataacacattatatccccacacttctcaagtggaaagtacctcaagtgacaaaattgaagactttaacagaatggaaaatgtggcaaaagaagttggaccactcgtatctcaaacagacatctttgaaggtagtgggtcagtaaccagcacaacattaatagaaattttaagtgacactggagcagaaggacccacggtggcacctctccctttctccacggacatcggacatcctcaaaatcagactgtcaggtgggcagaagaaatccagactagtagaccacaaaccataactgaacaagactctaacaagaattcttcaacagcagaaattaacgaaacaacaacctcatctactgattttctggctagagcttatggttttgaaatggccaaagaatttgttacatcagcaccaaaaccatctgacttgtattatgaaccttctggagaaggatctggagaagtggatattgttgattcatttcacacttctgcaactactcaggcaaccagacaagaaagcagcaccacatttgtttctgatgggtccctggaaaaacatcctgaggtgccaagcgctaaagctgttactgctgatggattcccaacagtttcagtgatgctgcctcttcattcagagcagaacaaaagctcccctgatccaactagcacactgtcaaatacagtgtcatatgagaggtccacagacggtagtttccaagaccgtttcagggaattcgaggattccaccttaaaacctaacagaaaaaaacccactgaaaatattatcatagacctggacaaagaggacaaggatttaatattgacaattacagagagtaccatccttgaaattctacctgagctgacatcggataaaaatactatcatagatattgatcatactaaacctgtgtatgaagacattcttggaatgcaaacagatatagatacagaggtaccatcagaaccacatgacagtaatgatgaaagtaatgatgacagcactcaagttcaagagatctatgaggcagctgtcaacctttctttaactgaggaaacatttgagggctctgctgatgttctggctagctacactcaggcaacacatgatgaatcaatgacttatgaagatagaagccaactagatcacatgggctttcacttcacaactgggatccctgctcctagcacagaaacagaattagacgttttacttcccacggcaacatccctgccaattcctcgtaagtctgccacagttattccagagattgaaggaataaaagctgaagcaaaagccctggatgacatgtttgaatcaagcactttgtctgatggtcaagctattgcagaccaaagtgaaataataccaacattgggccaatttgaaaggactcaggaggagtatgaagacaaaaaacatgctggtccttcttttcagccagaattctcttcaggagctgaggaggcattagtagaccatactccctatctaagtattgctactacccaccttatggatcagagtgtaacagaggtgcctgatgtgatggaaggatccaatcccccatattacactgatacaacattagcagtttcaacatttgcgaagttgtcttctcagacaccatcatctcccctcactatctactcaggcagtgaagcctctggacacacagagatcccccagcccagtgctctgccaggaatagacgtcggctcatctgtaatgtccccacaggattcttttaaggaaattcatgtaaatattgaagcaactttcaaaccatcaagtgaggaataccttcacataactgagcctccctctttatctcctgacacaaaattagaaccttcagaagatgatggtaaacctgagttattagaagaaatggaagcttctcccacagaacttattgctgtggaaggaactgagattctccaagatttccaaaacaaaaccgatggtcaagtttctggagaagcaatcaagatgtttcccaccattaaaacacctgaggctggaactgttattacaactgccgatgaaattgaattagaaggtgctacacagtggccacactctacttctgcttctgccacctatggggtcgaggcaggtgtggtgccttggctaagtccacagacttctgagaggcccacgctttcttcttctccagaaataaaccctgaaactcaagcagctttaatcagagggcaggattccacgatagcagcatcagaacagcaagtggcagcgagaattcttgattccaatgatcaggcaacagtaaaccctgtggaatttaatactgaggttgcaacaccaccattttcccttctggagacttctaatgaaacagatttcctgattggcattaatgaagagtcagtggaaggcacggcaatctatttaccaggacctgatcgctgcaaaatgaacccgtgccttaacggaggcacctgttatcctactgaaacttcctacgtatgcacctgtgtgccaggatacagcggagaccagtgtgaacttgattttgatgaatgtcactctaatccctgtcgtaatggagccacttgtgttgatggttttaacacattcaggtgcctctgccttccaagttatgttggtgcactttgtgagcaagataccgagacatgtgactatggctggcacaaattccaagggcagtgctacaaatactttgcccatcgacgcacatgggatgcagctgaacgggaatgccgtctgcagggtgcccatctcacaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagttgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtggcaggagtcgaggcgctga

SEQ ID NO: 25 - protein, 292 aa, Homo sapiens
MGPPSACPHRECIPWQGLLLTASLLTFWNAPTTAWLFIASAPFEVAEGENVHLSVVYLPENLYSYGWYKGKTVEPNQLIAAYVIDTHVRTPGPAYSGRETISPSGDLHFQNVTLEDTGYYNLQVTYRNSQIEQASHHLRVYESVAQPSIQASSTTVTEKGSVVLTCHTNNTGTSFQWIFNNQRLQVTKRMKLSWFNHVLTIDPIRQEDAGEYQCEVSNPVSSNRSDPLKLTVKYDNTLGILIGVLVGSLLVAALVCFLLLRKTGRASDQSDFREQQPPASTPGHGPSDSSIS

SEQ ID NO: 26 - DNA, 879 bp, Homo sapiens
atggggcccccctcagcttgtccccacagagaatgcatcccctggcaggggctcttgctcacagcctcacttttaactttctggaacgcacccaccactgcctggctctttattgcatcagcgccctttgaagttgctgaaggggagaatgttcatctctctgtggtttatctgcccgagaatctttacagctatggctggtacaaagggaaaacggtggagcccaaccagctaatcgcagcatatgtaatagacactcacgttaggactccagggcctgcatacagcggtcgagagacaatatcacccagtggagatctgcatttccagaacgtcaccctagaggacacgggatactacaacctacaagtcacatacagaaattctcagattgaacaggcatctcaccatctccgtgtatacgagtcagtggctcagccctccatccaagccagcagcaccacagtcacagagaagggctccgtggtcctgacctgccacacaaataacactggaacctctttccagtggattttcaacaaccagcgtctgcaggtcacgaagaggatgaagctgtcctggtttaaccatgtgctcaccatagaccccatcaggcaggaggacgctggggagtatcagtgtgaggtctccaacccagtcagctccaacaggagcgaccccctcaagctgactgtaaaatatgacaacactctaggcatcctgatcggggtcctggttgggagtcttctggtggctgcacttgtgtgtttcctgctcctccgaaaaactggcagggccagcgatcagagtgacttcagggagcagcagcccccagcctccacccccggccatggaccctctgacagctccatctcctag

SEQ ID NO: 27 - protein, 293 aa, Homo sapiens
MGPPSACPHRECIPWQGLLLTASLLTFWNAPTTAWLFIASAPFEVAEGENVHLSVVYLPENLYSYGWYKGKTVEPNQLIAAYVIDTHVRTPGPAYSGRETISPSGDLHFQNVTLEDTGYYNLQVTYRNSQIEQASHHLRVYESVAQPSIQASSTTVTEKGSVVLTCHTNNTGTSFQWIFNNQRLQVTKRMKLSWFNHVLTIDPIRQEDAGEYQCEVSNPVSSNRSDPLKLTVKSDDNTLGILIGVLVGSLLVAALVCFLLLRKTGRASDQSDFREQQPPASTPGHGPSDSSIS

SEQ ID NO: 28 - DNA, 882 bp, Homo sapiens
atggggcccccctcagcttgtccccacagagaatgcatcccctggcaggggctcttgctcacagcctcacttttaactttctggaacgcacccaccactgcctggctctttattgcatcagcgccctttgaagttgctgaaggggagaatgttcatctctctgtggtttatctgcccgagaatctttacagctatggctggtacaaagggaaaacggtggagcccaaccagctaatcgcagcatatgtaatagacactcacgttaggactccagggcctgcatacagcggtcgagagacaatatcacccagtggagatctgcatttccagaacgtcaccctagaggacacgggatactacaacctacaagtcacatacagaaattctcagattgaacaggcatctcaccatctccgtgtatacgagtcagtggctcagccctccatccaagccagcagcaccacagtcacagagaagggctccgtggtcctgacctgccacacaaataacactggaacctctttccagtggattttcaacaaccagcgtctgcaggtcacgaagaggatgaagctgtcctggtttaaccatgtgctcaccatagaccccatcaggcaggaggacgctggggagtatcagtgtgaggtctccaacccagtcagctccaacaggagcgaccccctcaagctgactgtaaaatcagatgacaacactctaggcatcctgatcggggtcctggttgggagtcttctggtggctgcacttgtgtgtttcctgctcctccgaaaaactggcagggccagcgatcagagtgacttcagggagcagcagcccccagcctccacccccggccatggaccctctgacagctccatctcctag

SEQ ID NO: 29 - protein, 235 aa, Homo sapiens
MGPPSACPHRECIPWQGLLLTASLLTFWNAPTTAWLFIASAPFEVAEGENVHLSVVYLPENLYSYGWYKGKTVEPNQLIAAYVIDTHVRTPGPAYSGRETISPSGDLHFQNVTLEDTGYYTLQVTYRNSQIEQASHHLRVYESVAQPSIQASSTTVTEKGSVVLTCHTNNTGTSFQWIFNNQRLQVTKRMKLSWFNHMLTIDPIRQEDAGEYQCEVSNPVSSNRSDPLKLTVKCE

SEQ ID NO: 30 - DNA, 708 bp, Homo sapiens
atggggcccccctcagcttgtccccacagagaatgcatcccctggcaggggctcttgctcacagcctcacttttaactttctggaacgcacccaccactgcctggctctttattgcatcagcgccctttgaagttgctgaaggggagaatgttcatctctctgtggtttatctgcccgagaatctttacagctatggctggtacaaagggaaaacggtggagcccaaccagctaatcgcagcatatgtaatagacactcacgttaggactccagggcctgcatacagcggtcgagagacaatatcacccagtggagatctgcatttccagaacgtcaccctagaggacacgggatactacaccctacaagtcacatacagaaattctcagattgaacaggcatctcaccatctccgtgtatacgagtcagtggctcagccctccatccaagccagcagcaccacagtcacagagaagggctccgtggtcctgacctgccacacaaataacactggaacctctttccagtggattttcaacaaccagcgtctgcaggtcacgaagaggatgaagctgtcctggtttaaccatatgctcaccatagaccccatcaggcaggaggacgctggggagtatcagtgtgaggtctccaacccagtcagctccaacaggagcgaccccctcaagctgactgtaaaatgtgagtga

SEQ ID NO: 31 - protein, 376 aa, Homo sapiens
MQWTSLLLLAGLFSLSQAQYEDDPHWWFHYLRSQQSTYYDPYDPYPYETYEPYPYGVDEGPAYTYGSPSPPDPRDCPQECDCPPNFPTAMYCDNRNLKYLPFVPSRMKYVYFQNNQITSIQEGVFDNATGLLWIALHGNQITSDKVGRKVFSKLRHLERLYLDHNNLTRMPGPLPRSLRELHLDHNQISRVPNNALEGLENLTALYLQHNEIQEVGSSMRGLRSLILLDLSYNHLRKVPDGLPSALEQLYMEHNNVYTVPDSYFRGAPKLLYVRLSHNSLTNNGLASNTFNSSSLLELDLSYNQLQKIPPVNTNLENLYLQGNRINEFSISSFCTVVDVVNFSKLQVLRLDGNEIKRSAMPADAPLCLRLASLIEI

SEQ ID NO: 32 - DNA, 1131 bp, Homo sapiens
atgcagtggacctccctcctgctgctggcagggctcttctccctctcccaggcccagtatgaagatgaccctcattggtggttccactacctccgcagccagcagtccacctactacgatccctatgacccttacccgtatgagacctacgagccttacccctatggggtggatgaagggccagcctacacctacggctctccatcccctccagatccccgcgactgcccccaggagtgcgactgcccacccaacttccccacggccatgtactgtgacaatcgcaacctcaagtacctgcccttcgttccctcccgcatgaagtatgtgtacttccagaacaaccagatcacctccatccaggaaggcgtctttgacaatgccacagggctgctctggattgctctccacggcaaccagatcaccagtgataaggtgggcaggaaggtcttctccaagctgaggcacctggagaggctgtacctggaccacaacaacctgacccggatgcccggtcccctgcctcgatccctgagagagctccatctcgaccacaaccagatctcacgggtccccaacaatgctctggaggggctggagaacctcacggccttgtacctccaacacaatgagatccaggaagtgggcagttccatgaggggcctccggtcactgatcttgctggacctgagttataaccaccttcggaaggtgcctgatgggctgccctcagctcttgagcagctgtacatggagcacaacaatgtctacaccgtccccgatagctacttccggggggcgcccaagctgctgtatgtgcggctgtcccacaacagtctaaccaacaatggcctggcctccaacaccttcaattccagcagcctccttgagctagacctctcctacaaccagctgcagaagatccccccagtcaacaccaacctggagaacctctacctccaaggcaataggatcaatgagttctccatcagcagcttctgcaccgtggtggacgtcgtgaacttctccaagctgcaggtgctgcgcctggacgggaacgagatcaagcgcagcgccatgcctgccgacgcgcccctctgcctgcgccttgccagcctcatcgagatctga

SEQ ID NO: 33 - protein, 1496 aa, Homo sapiens
SRPWWLRASERPSAPSAMAKRSRGPGRRCLLALVLFCAWGTLAVVAQKPGAGCPSRCLCFRTTVRCMHLLLEAVPAVAPQTSILDLRFNRIREIQPGAFRRLRNLNTLLLNNNQIKRIPSGAFEDLENLKYLYLYKNEIQSIDRQAFKGLASLEQLYLHFNQIETLDPDSFQHLPKLERLFLHNNRITHLVPGTFNHLESMKRLRLDSNTLHCDCEILWLADLLKTYAESGNAQAAAICEYPRRIQGRSVATITPEELNCERPRITSEPQDADVTSGNTVYFTCRAEGNPKPEIIWLRNNNELSMKTDSRLNLLDDGTLMIQNTQETDQGIYQCMAKNVAGEVKTQEVTLRYFGSPARPTFVIQPQNTEVLVGESVTLECSATGHPPPRISWTRGDRTPLPVDPRVNITPSGGLYIQNVVQGDSGEYACSATNNIDSVHATAFIIVQALPQFTVTPQDRVVIEGQTVDFQCEAKGNPPPVIAWTKGGSQLSVDRRHLVLSSGTLRISGVALHDQGQYECQAVNIIGSQKVVAHLTVQPRVTPVFASIPSDTTVEVGANVQLPCSSQGEPEPAITWNKDGVQVTESGKFHISPEGFLTINDVGPADAGRYECVARNTIGSASVSMVLSVNVPDVSRNGDPFVATSIVEAIATVDRAINSTRTHLFDSRPRSPNDLLALFRYPRDPYTVEQARAGEIFERTLQLIQEHVQHGLMVDLNGTSYHYNDLVSPQYLNLIANLSGCTAHRRVNNCSDMCFHQKYRTHDGTCNNLQHPMWGASLTAFERLLKSVYENGFNTPRGINPHRLYNGHALPMPRLVSTTLIGTETVTPDEQFTHMLMQWGQFLDHDLDSTVVALSQARFSDGQHCSNVCSNDPPCFSVMIPPNDSRARSGARCMFFVRSSPVCGSGMTSLLMNSVYPREQINQLTSYIDASNVYGSTEHEARSIRDLASHRGLLRQGIVQRSGKPLLPFATGPPTECMRDENESPIPCFLAGDHRANEQLGLTSMHTLWFREHNRIATELLKLNPHWDGDTIYYETRKIVGAEIQHITYQHWLPKILGEVGMRTLGEYHGYDPGINAGIFNAFATAAFRFGHTLVNPLLYRLDENFQPIAQDHLPLHKAFFSPFRIVNEGGIDPLLRGLFGVAGKMRVPSQLLNTELTERLFSMAHTVALDLAAINIQRGRDHGIPPYHDYRVYCNLSAAHTFEDLKNEIKNPEIREKLKRLYGSTLNIDLFPALVVEDLVPGSRLGPTLMCLLSTQFKRLRDGDRLWYENPGVFSPAQLTQIKQTSLARILCDNADNITRVQSDVFRVAEFPHGYGSCDEIPRVDLRVWQDCCEDCRTRGQFNAFSYHFRGRRSLEFSYQEDKPTKKTRPRKIPSVGRQGEHLSNSTSAFSTRSDASGTNDFREFVLEMQKTITDLRTQIKKLESRLSTTECVDAGGESHANNTKWKKDACTICECKDGQVTCFVEACPPATCAVPVNIPGACCPVCLQKRAEEKP

SEQ ID NO: 34 - DNA, 4491 bp, Homo sapiens
agccggccgtggtggctccgtgcgtccgagcgtccgtccgcgccgtcggccatggccaagcgctccaggggccccgggcgccgctgcctgttggcgctcgtgctgttctgcgcctgggggacgctggccgtggtggcccagaagccgggcgcagggtgtccgagccgctgcctgtgcttccgcaccaccgtgcgctgcatgcatctgctgctggaggccgtgcccgccgtggcgccgcagacctccatcctagatcttcgctttaacagaatcagagagatccaacctggggcattcaggcggctgaggaacttgaacacattgcttctcaataataatcagatcaagaggatacctagtggagcatttgaagacttggaaaatttaaaatatctctatctgtacaagaatgagatccagtcaattgacaggcaagcatttaagggacttgcctctctagagcaactatacctgcactttaatcagatagaaactttggacccagattcgttccagcatctcccgaagctcgagaggctatttttgcataacaaccggattacacatttagttccagggacatttaatcacttggaatctatgaagagattgcgactggactcaaacacacttcactgcgactgtgaaatcctgtggttggcggatttgctgaaaacctacgcggagtcggggaacgcgcaggcagcggccatctgtgaatatcccagacgcatccagggacgctcagtggcaaccatcaccccggaagagctgaactgtgaaaggccccggatcacctccgagccccaggacgcagatgtgacctcggggaacaccgtgtacttcacctgcagagccgaaggcaaccccaagcctgagatcatctggctgcgaaacaataatgagctgagcatgaagacagattcccgcctaaacttgctggacgatgggaccctgatgatccagaacacacaggagacagaccagggtatctaccagtgcatggcaaagaacgtggccggagaggtgaagacgcaagaggtgaccctcaggtacttcgggtctccagctcgacccacttttgtaatccagccacagaatacagaggtgctggttggggagagcgtcacgctggagtgcagcgccacaggccaccccccgccgcggatctcctggacgagaggtgaccgcacacccttgccagttgacccgcgggtgaacatcacgccttctggcgggctttacatacagaacgtcgtacagggggacagcggagagtatgcgtgctctgcgaccaacaacattgacagcgtccatgccaccgctttcatcatcgtccaggctcttcctcagttcactgtgacgcctcaggacagagtcgttattgagggccagaccgtggatttccagtgtgaagccaagggcaacccgccgcccgtcatcgcctggaccaagggagggagccagctctccgtggaccggcggcacctggtcctgtcatcgggaacacttagaatctctggtgttgccctccacgaccagggccagtacgaatgccaggctgtcaacatcatcggctcccagaaggtcgtggcccacctgactgtgcagcccagagtcaccccagtgtttgccagcattcccagcgacacaacagtggaggtgggcgccaatgtgcagctcccgtgcagctcccagggcgagcccgagccagccatcacctggaacaaggatggggttcaggtgacagaaagtggaaaatttcacatcagccctgaaggattcttgaccatcaatgacgttggccctgcagacgcaggtcgctatgagtgtgtggcccggaacaccattgggtcggcctcggtgagcatggtgctcagtgtgaacgttcctgacgtcagtcgaaatggagatccgtttgtagctacctccatcgtggaagcgattgcgactgttgacagagctataaactcaacccgaacacatttgtttgacagccgtcctcgttctccaaatgatttgctggccttgttccggtatccgagggatccttacacagttgaacaggcacgggcgggagaaatctttgaacggacattgcagctcattcaggagcatgtacagcatggcttgatggtcgacctcaacggaacaagttaccactacaacgacctggtgtctccacagtacctgaacctcatcgcaaacctgtcgggctgtaccgcccaccggcgcgtgaacaactgctcggacatgtgcttccaccagaagtaccggacgcacgacggcacctgtaacaacctgcagcaccccatgtggggcgcctcgctgaccgccttcgagcgcctgctgaaatccgtgtacgagaatggcttcaacacccctcggggcatcaacccccaccgactgtacaacgggcacgcccttcccatgccgcgcctggtgtccaccaccctgatcgggacggagaccgtcacacccgacgagcagttcacccacatgctgatgcagtggggccagttcctggaccacgacctcgactccacggtggtggccctgagccaggcacgcttctccgacggacagcactgcagcaacgtgtgcagcaacgaccccccctgcttctctgtcatgatcccccccaatgactcccgggccaggagcggggcccgctgcatgttcttcgtgcgctccagccctgtgtgcggcagcggcatgacttcgctgctcatgaactccgtgtacccgcgggagcagatcaaccagctcacctcctacatcgacgcatccaacgtgtacgggagcacggagcatgaggcccgcagcatccgcgacctggccagccaccgcggcctgctgcggcagggcatcgtgcagcggtccgggaagccgctgctccccttcgccaccgggccgcccacggagtgcatgcgggacgagaacgagagccccatcccctgcttcctggccggggaccaccgcgccaacgagcagctgggcctgaccagcatgcacacgctgtggttccgcgagcacaaccgcattgccacggagctgctcaagctgaacccgcactgggacggcgacaccatctactatgagaccaggaagatcgtgggtgcggagatccagcacatcacctaccagcactggctcccgaagatcctgggggaggtgggcatgaggacgctgggagagtaccacggctacgaccccggcatcaatgctggcatcttcaacgccttcgccaccgcggccttcaggtttggccacacgcttgtcaacccactgctttaccggctggacgagaacttccagcccattgcacaagatcacctcccccttcacaaagctttcttctctcccttccggattgtgaatgagggcggcatcgatccgcttctcagggggctgttcggggtggcggggaaaatgcgtgtgccctcgcagctgctgaacacggagctcacggagcggctgttctccatggcacacacggtggctctggacctggcggccatcaacatccagcggggccgggaccacgggatcccaccctaccacgactacagggtctactgcaatctatcggcggcacacacgttcgaggacctgaaaaatgagattaaaaaccctgagatccgggagaaactgaaaaggttgtatggctcgacactcaacatcgacctgtttccggcgctcgtggtggaggacctggtgcctggcagccggctgggccccaccctgatgtgtcttctcagcacacagttcaagcgcctgcgagatggggacaggttgtggtatgagaaccctggggtgttctccccggcccagctgactcagatcaagcagacgtcgctggccaggatcctatgcgacaacgcggacaacatcacccgggtgcagagcgacgtgttcagggtggcggagttccctcacggctacggcagctgtgacgagatccccagggtggacctccgggtgtggcaggactgctgtgaagactgtaggaccagggggcagttcaatgccttttcctatcatttccgaggcagacggtctcttgagttcagctaccaggaggacaagccgaccaagaaaacaagaccacggaaaatacccagtgttgggagacagggggaacatctcagcaacagcacctcagccttcagcacacgctcagatgcatctgggacaaatgacttcagagagtttgttctggaaatgcagaagaccatcacagacctcagaacacagataaagaaacttgaatcacggctcagtaccacagagtgcgtggatgccgggggcgaatctcacgccaacaacaccaagtggaaaaaagatgcatgcaccatttgtgaatgcaaagacgggcaggtcacctgcttcgtggaagcttgcccccctgccacctgtgctgtccccgtgaacatcccaggggcctgctgtccagtctgcttacagaagagggcggaggaaaagccctag

SEQ ID NO: 35 - protein, 613 aa, Homo sapiens
MRAPGALLARMSRLLLLLLLKVSASSALGVAPASRNETCLGESCAPTVIQRRGRDAWGPGNSARDVLRARAPREEQGAAFLAGPSWDLPAAPGRDPAAGRGAEASAAGPPGPPTRPPGPWRWKGARGQEPSETLGRGNPTALQLFLQISEEEEKGPRGAGISGRSQEQSVKTVPGASDLFYWPRRAGKLQGSHHKPLSKTANGLAGHEGWTIALPGRALAQNGSLGEGIHEPGGPRRGNSTNRRVRLKNPFYPLTQESYGAYAVMCLSVVIFGTGIIGNLAVMCIVCHNYYMRSISNSLLANLAFWDFLIIFFCLPLVIFHELTKKWLLEDFSCKIVPYIEVASLGVTTFTLCALCIDRFRAATNVQMYYEMIENCSSTTAKLAVIWVGALLLALPEVVLRQLSKEDLGFSGRAPAERCIIKISPDLPDTIYVLALTYDSARLWWYFGCYFCLPTLFTITCSLVTARKIRKAEKACTRGNKRQIQLESQMNCTVVALTILYGFCIIPENICNIVTAYMATGVSQQTMDLLNIISQFLLFFKSCVTPVLLFCLCKPFSRAFMECCCCCCEECIQKSSTVTSDDNDNEYTTELELSPFSTIRREMSTFASVGTHC

SEQ ID NO: 36 - DNA, 1842 bp, Homo sapiens
atgcgagccccgggcgcgcttctcgcccgcatgtcgcggctactgcttctgctactgctcaaggtgtctgcctcttctgccctcggggtcgcccctgcgtccagaaacgaaacttgtctgggggagagctgtgcacctacagtgatccagcgccgcggcagggacgcctggggaccgggaaattctgcaagagacgttctgcgagcccgagcacccagggaggagcagggggcagcgtttcttgcgggaccctcctgggacctgccggcggccccgggccgtgacccggctgcaggcagaggggcggaggcgtcggcagccggacccccgggacctccaaccaggccacctggcccctggaggtggaaaggtgctcggggtcaggagccttctgaaactttggggagagggaaccccacggccctccagctcttccttcagatctcagaggaggaagagaagggtcccagaggcgctggcatttccgggcgtagccaggagcagagtgtgaagacagtccccggagccagcgatcttttttactggccaaggagagccgggaaactccagggttcccaccacaagcccctgtccaagacggccaatggactggcggggcacgaagggtggacaattgcactcccgggccgggcgctggcccagaatggatccttgggtgaaggaatccatgagcctgggggtccccgccggggaaacagcacgaaccggcgtgtgagactgaagaaccccttctacccgctgacccaggagtcctatggagcctacgcggtcatgtgtctgtccgtggtgatcttcgggaccggcatcattggcaacctggcggtgatgtgcatcgtgtgccacaactactacatgcggagcatctccaactccctcttggccaacctggccttctgggactttctcatcatcttcttctgccttccgctggtcatcttccacgagctgaccaagaagtggctgctggaggacttctcctgcaagatcgtgccctatatagaggtcgcttctctgggagtcaccactttcaccttatgtgctctgtgcatagaccgcttccgtgctgccaccaacgtacagatgtactacgaaatgatcgaaaactgttcctcaacaactgccaaacttgctgttatatgggtgggagctctattgttagcacttccagaagttgttctccgccagctgagcaaggaggatttggggtttagtggccgagctccggcagaaaggtgcattattaagatctctcctgatttaccagacaccatctatgttctagccctcacctacgacagtgcgagactgtggtggtattttggctgttacttttgtttgcccacgcttttcaccatcacctgctctctagtgactgcgaggaaaatccgcaaagcagagaaagcctgtacccgagggaataaacggcagattcaactagagagtcagatgaactgtacagtagtggcactgaccattttatatggattttgcattattcctgaaaatatctgcaacattgttactgcctacatggctacaggggtttcacagcagacaatggacctccttaatatcatcagccagttccttttgttctttaagtcctgtgtcaccccagtcctccttttctgtctctgcaaacccttcagtcgggccttcatggagtgctgctgctgttgctgtgaggaatgcattcagaagtcttcaacggtgaccagtgatgacaatgacaacgagtacaccacggaactcgaactctcgcctttcagtaccatacgccgtgaaatgtccacttttgcttctgtcggaactcattgctga

SEQ ID NO: 37 - protein, 1471 aa, Homo sapiens
PEMTGVTVSGLTPARTYQFRVCAVNEVGRGQYSAETSRLMLPEEPPSAPPKNIVASGRTNQSIMVQWQPPPETEHNGVLRGYILRYRLAGLPGEYQQRNITSPEVNYCLVTDLIIWTQYEIQVAAYNGAGLGVFSRAVTEYTLQGVPTAPPQNVQTEAVNSTTIQFLWNPPPQQFINGINQGYKLLAWPADAPEAVTVVTIAPDFHGVHHGHITNLKKFTAYFTSVLCFTTPGDGPPSTPQLVWTQEDKPGAVGHLSFTEILDTSLKVSWQEPLEKNGIITGYQISWEVYGRNNSRLTHTLNSTTHEYKIQGLSSLTTYTIDVAAVTAVGTGLVTSSTISSGVPPDLPGAPSNLVISNISPRSATLQFRPGYDGKTSISRWIVEGQVGAIGDEEEWVTLYEEENEPDAQMLEIPNLTPYTHYRFRMKQVNIVGPSPYSPSSRVIQTLQAPPDVAPTSVTVRTASETSLRLRWVPLPDSQYNGNPESVGYRIKYWRSDLQSSAVAQVVSDRLEREFTIEELEEWMEYELQMQAFNAVGAGPWSEVVRGRTRESVPSAAPENVSAEAVSSTQILLTWTSVPEQDQNGLILGYKILFRAKDLDPEPRSHIVRGNHTQSALLAGLRKFVLYELQVLAFTRIGNGVPSTPLILERTKDDAPGPPVRLVFPEVRLTSVRIVWQPPEEPNGIILGYQIAYRLASSSPHTFTTVEVGATVRQFTATDLAPESAYIFRLSAKTRQGWGEPLEATVITTEKRERPAPPRELLVPQAEVTARSLRLQWVPGSDGASPIRYFTMQVRELPRGEWQTYSSSISHEATACVVDRLRPFTSYKLRLKATNDIGDSDFSSETEAVTTLQDVPGEPPGSVSATPHTTSSVLIQWQPPRDESLNGLLQGYRIYYRELEYEAGSGTEAKTLKNPIALRAELTDLKKYRRYEVIMTAYNIIGESPASAPVEVFVGEAAPAMAPQNVQVTPLTASQLEVTWDPPPPESQNGNIQGYKIYYWEADSQNETEKMKVLFLPEPVVRLKNLTSHTKYLVSISAFNAAGDGPKSDPQQGRTHQAAPGAPSFLAFSEITSTTLNVSWGEPAAANGILQGYRVVYEPLAPVQGVSKVVTVEVRGNWQRWLKVRDLTKGVTYFFRVQARTITYGPELQANITAGPAEGSPGSPRDVLVTKSASELTLQWTEGHSGDTPTTGYVIEARPSDEGLWDMFVKDIPRSATSYTLSLDKLRQGVTYEFRVVAVNEAGYGEPSNPSTAVSAQVEAPFYEEWWFLLVMALSSLIVILLVVFALVLHGQNKKYKNCSTGKGISTMEESVTLDNGGFAALELSSRHLNVKSTFSKKNGTRSPPRPSPGGLHYSDEDICNKYNGAVLTESVSLKEKSADASESEATDSDYEDALPKHSFVNHYMSDPTYYNSWKRRAQGRAPAPHRYEAVAGSEAGAQLHPVITTQSAGGVYTPAGPGARTPLTGFSSFV

SEQ ID NO: 38 - DNA, 4417 bp, Homo sapiens
ccctgagatgacaggcgtcaccgtgagtggcctgactccggctcgtacctatcaattccgggtgtgcgcggtgaatgaagtgggcaggggccagtacagtgccgagacaagcaggttgatgctacctgaagaaccacccagtgctcccccgaaaaatatagtggccagtgggcggactaatcagtccattatggtccagtggcagccacccccagaaacagagcacaacggggtgttgcgtggatacatcctcaggtaccgcctggctggccttcccggagagtaccagcagcggaacatcaccagcccggaggtgaactactgcctggtgacagacctgatcatctggacacagtatgagatacaggtggcggcgtacaacggggccggtctgggcgtcttcagcagggcagtgaccgagtacaccttgcagggagtgcccaccgcgcccccgcagaacgtgcagacggaagccgtgaactccaccaccattcagttcctgtggaaccctccgcctcagcagtttatcaatggcatcaaccagggatacaagcttctggcatggccggcagatgcccccgaggctgtcactgtggtcactattgccccagatttccacggagtccaccatggacacataacgaacctgaagaagtttaccgcctacttcacttccgttctgtgcttcaccacccctggggacgggcctcccagcacacctcagctggtctggactcaggaagacaaaccaggagctgtgggacatctgagtttcacagagatcttggacacatctctcaaggtcagctggcaggagcccctggagaaaaatggcatcattactggctatcagatctcttgggaagtgtacggcaggaacaactctcgtctcacgcacaccctgaacagcacgacgcacgagtacaagatccaaggcctctcatctctcaccacctacaccatcgacgtggccgctgtgactgccgtgggcactggcctggtgacttcatccaccatttcttctggagtgcccccagaccttcctggtgccccatccaacctggtcatttccaacatcagccctcgctccgccacccttcagttccggccaggctatgacgggaaaacgtccatctccaggtggattgttgaggggcaggtgggagctatcggcgacgaggaggagtgggtcaccctctatgaagaggagaatgagcctgatgcccagatgctggagatcccaaacctcacaccctacactcactacagatttcgaatgaagcaagtgaacattgttgggccgagcccctacagtccgtcttcccgggtcatccagaccctgcaggccccacccgacgtggctccaaccagcgtcacggtccgtactgccagtgagaccagcctgcggcttcgctgggtgcccctgccggattctcagtacaacgggaaccccgagtccgtgggctacaggattaagtactggcgctcagacctccagtcctcagcagtggcccaagtcgtcagtgaccggctggagagagaattcaccatcgaggagctggaggagtggatggaatacgagctgcagatgcaggccttcaacgccgtcggggctgggccgtggagcgaggtggtgcggggccggacgcgggagtcagttccttcagccgcccctgagaacgtgtcagccgaggctgtcagctcgacccagattttactgacatggacatccgtgccggaacaggaccagaatgggctcatactgggctacaagatcctgttccgggccaaagacctggatcccgagcccaggagccacatcgtgcgagggaaccacacgcagtcggccctgctggcaggcctgcgcaagttcgtgctctacgagctccaggtgctggcgttcacccgcatcgggaacggggtccccagcacgcccctcatcctggagcgcaccaaagacgatgccccaggcccaccagtgaggctcgtgttccccgaagtgagactcacctctgtgcggatagtgtggcaacctccggaggagcccaacggcatcatcctggggtaccagattgcctaccgcctggccagcagcagcccccacaccttcaccaccgtggaggtcggcgccacagtgaggcagttcacagccaccgacctggccccggagtccgcatacatcttcaggctgtccgccaagacgaggcagggctggggggagccactggaggccaccgtcatcaccaccgagaagagagagcggccggcaccccccagagagctcctggtgccccaggcagaagtgaccgcacgcagcctccggctccagtgggtcccgggcagcgacggggcctcccccatccggtacttcaccatgcaggtgcgagagctgcctcggggtgagtggcagacctactcctcgtccatcagccatgaggccacagcatgcgtcgttgacagactgaggcccttcacctcctacaagctgcgcctgaaagccaccaacgacattggggacagtgacttcagttcagagacagaggcggtgaccacgctgcaggatgttccaggagagcccccgggatctgtctcagcgacgccacacaccacgtcctctgtcctgatacagtggcagcctccgagggacgaaagcctgaacggccttcttcagggatacaggatctactacagggagctggagtatgaagccgggtcaggcaccgaggccaagacgctcaaaaaccctatagctttacgtgctgagctcacagatttaaagaagtaccggcgctatgaagtaataatgaccgcctataacatcatcggcgagagcccagccagcgcgcccgtggaggtctttgtcggcgaggctgccccggccatggccccgcagaacgtgcaggtgaccccactcacggccagccagctggaggtcacgtgggacccaccacccccggagagccagaatgggaacatccaaggctacaagatttactactgggaggcagacagccagaacgaaacggagaaaatgaaggtcctcttcctccccgagcccgtggtgaggctgaagaacctgaccagccataccaagtacctggtcagcatatcagccttcaacgccgccggagatggacctaagagtgacccccagcaggggcgcacccaccaggccgcccctggggcccccagctttctggcgttctcagaaataacctccaccacgctcaacgtgtcctggggcgagcctgcggcggccaacggcatcctgcagggctatcgggtggtgtacgagcccttggcccctgtacaaggggtgagcaaggtggtgaccgtggaagtgagagggaactggcagcgctggctgaaggtgcgggacctcaccaagggagtgacctatttcttccgtgtccaagcgcggaccatcacctacgggcccgagctccaagccaatatcacagccgggccagccgagggatccccgggctcgcctagagatgtcctggtcaccaagtccgcctctgaactgacgctgcagtggactgagggacactctggcgacacacctaccacgggctatgtgatcgaggcccggccctcagatgaaggcttatgggacatgtttgtgaaggacatcccgcggagcgccacatcctacaccctcagcctggataagctccggcaaggagtgacttacgagttccgggtggtggctgtgaatgaggcgggctacggggagcccagcaacccctccacggctgtgtcagctcaagtggaagccccattctacgaggagtggtggttcctcctggtgatggctctgtccagcctgatcgtcatcctgctggtggtgttcgccctcgtcctgcacgggcagaataagaagtataagaactgcagcacaggaaaggggatctccaccatggaggagtctgtgaccctggacaacggaggatttgctgccctggagctcagcagccgccacctcaatgtcaagagcaccttctccaagaagaacgggaccaggtccccaccccggcctagccccggcggcctgcactactcagacgaggacatctgcaacaagtacaacggcgccgtgctgaccgagagcgtgagcctcaaggagaagtcggcagatgcatcagaatctgaggccacggactctgactacgaggacgcgctgcccaagcactccttcgtgaaccactacatgagcgaccccacctactacaactcatggaagcgcagggcccagggccgcgcacctgcgccgcacaggtacgaggcggtggcgggctccgaggcgggcgcgcagctgcacccggtcatcaccacgcagagcgcgggcggcgtctacacccccgctggccccggcgcgcgaactccgctcaccggcttctcctccttcgtgtga

SEQ ID NO: 39 - protein, 2506 aa, Homo sapiens
MARFGDEMPARYGGGGSGAAAGVVVGSGGGRGAGGSRQGGQPGAQRMYKQSMAQRARTMALYNPIPVRQNCLTVNRSLFLFSEDNVVRKYAKKITEWPPFEYMILATIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREDPYGRESDHHAREGSLEQPGFWEGEAERGKAGDPHRRHVHRQGGSRESRSGSPRTGANGEHRRHRAHRSPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPSPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAAWGRMPYLDMYQMLRHMSPPLGLGKKCPARVAYKRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQRRRGRPRGNNLSTISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQGSSSVSGSPAPSTSGTSTPRRGRRQLPQTPSTPRPHVSYSPVIRKAGGSGPPQQQQQQQQQQQQQAVARPGRAATSGPRRYPGPTAEPLAGDRPPTGGHSSGRSPRMERRVPGPARSESPRACRHGGARWPASGPHVSEGPPGPRHHGYYRGSDYDEADGPGSGGGEEAMAGAYDAPPPVRHASSGATGRSPRTPRASGPACASPSRHGRRLPNGYYPAHGLARPRGPGSRKGLHEPYSESDDDWC

SEQ ID NO: 40 - DNA, 7521 bp, Homo sapiens
atggcccgcttcggagacgagatgccggcccgctacgggggaggaggctccggggcagccgccggggtggtcgtgggcagcggaggcgggcgaggagccgggggcagccggcagggcgggcagcccggggcgcaaaggatgtacaagcagtcaatggcgcagagagcgcggaccatggcactctacaaccccatccccgtccgacagaactgcctcacggttaaccggtctctcttcctcttcagcgaagacaacgtggtgagaaaatacgccaaaaagatcaccgaatggcctccctttgaatatatgattttagccaccatcatagcgaattgcatcgtcctcgcactggagcagcatctgcctgatgatgacaagaccccgatgtctgaacggctggatgacacagaaccatacttcattggaattttttgtttcgaggctggaattaaaatcattgcccttgggtttgccttccacaaaggctcctacttgaggaatggctggaatgtcatggactttgtggtggtgctaacgggcatcttggcgacagttgggacggagtttgacctacggacgctgagggcagttcgagtgctgcggccgctcaagctggtgtctggaatcccaagtttacaagtcgtcctgaagtcgatcatgaaggcgatgatccctttgctgcagatcggcctcctcctattttttgcaatccttatttttgcaatcatagggttagaattttatatgggaaaatttcataccacctgctttgaagaggggacagatgacattcagggtgagtctccggctccatgtgggacagaagagcccgcccgcacctgccccaatgggaccaaatgtcagccctactgggaggggcccaacaacgggatcactcagttcgacaacatcctgtttgcagtgctgactgttttccagtgcataaccatggaagggtggactgatctcctctacaatagcaacgatgcctcagggaacacttggaactggttgtacttcatccccctcatcatcatcggctccttttttatgctgaaccttgtgctgggtgtgctgtcaggggagtttgccaaagaaagggaacgggtggagaaccggcgggcttttctgaagctgaggcggcaacaacagattgaacgtgagctcaatgggtacatggaatggatctcaaaagcagaagaggtgatcctcgccgaggatgaaactgacggggagcagaggcatccctttgatgctctgcggagaaccaccataaagaaaagcaagacagatttgctcaaccccgaagaggctgaggatcagctggctgatatagcctctgtgggttctcccttcgcccgagccagcattaaaagtgccaagctggagaactcgaccttttttcacaaaaaggagaggaggatgcgtttctacatccgccgcatggtcaaaactcaggccttctactggactgtactcagtttggtagctctcaacacgctgtgtgttgctattgttcactacaaccagcccgagtggctctccgacttcctttactatgcagaattcattttcttaggactctttatgtccgaaatgtttataaaaatgtacgggcttgggacgcggccttacttccactcttccttcaactgctttgactgtggggttatcattgggagcatcttcgaggtcatctgggctgtcataaaacctggcacatcctttggaatcagcgtgttacgagccctcaggttattgcgtattttcaaagtcacaaagtactgggcatctctcagaaacctggtcgtctctctcctcaactccatgaagtccatcatcagcctgttgtttctccttttcctgttcattgtcgtcttcgcccttttgggaatgcaactcttcggcggccagtttaatttcgatgaagggactcctcccaccaacttcgatacttttccagcagcaataatgacggtgtttcagatcctgacgggcgaagactggaacgaggtcatgtacgacgggatcaagtctcaggggggcgtgcagggcggcatggtgttctccatctatttcattgtactgacactctttgggaactacaccctcctgaatgtgttcttggccatcgctgtggacaatctggccaacgcccaggagctcaccaaggacgagcaagaggaagaagaagcagcgaaccagaaacttgccctacagaaagccaaggaggtggcagaagtgagtcctctgtccgcggccaacatgtctatagctgtgaaagagcaacagaagaatcaaaagccagccaagtccgtgtgggagcagcggaccagtgagatgcgaaagcagaacttgctggccagccgggaggccctgtataacgaaatggacccggacgagcgctggaaggctgcctacacgcggcacctgcggccagacatgaagacgcacttggaccggccgctggtggtggacccgcaggagaaccgcaacaacaacaccaacaagagccgggcggccgagcccaccgtggaccagcgcctcggccagcagcgcgccgaggacttcctcaggaaacaggcccgctaccacgatcgggcccgggaccccagcggctcggcgggcctggacgcacggaggccctgggcgggaagccaggaggccgagctgagccgggaggacccctacggccgcgagtcggaccaccacgcccgggagggcagcctggagcaacccgggttctgggagggcgaggccgagcgaggcaaggccggggacccccaccggaggcacgtgcaccggcaggggggcagcagggagagccgcagcgggtccccgcgcacgggcgcgaacggggagcatcgacgtcatcgcgcgcaccgcagccccggggaggagggtccggaggacaaggcggagcggagggcgcggcaccgcgagggcagccggccggcccggggcggcgagggcgagggcgagggccccgacgggggcgagcgcaggagaaggcaccggcatggcgctccagccacgtacgagggggacgcgcggagggaggacaaggagcggaggcatcggaggaggaaagagaaccagggctccggggtccctgtgtcgggccccaacctgtcaaccacccggccaatccagcaggacctgggccgccaagacccacccctggcagaggatattgacaacatgaagaacaacaagctggccaccgcggagtcggccgctccccacggcagccttggccacgccggcctgccccagagcccagccaagatgggaaacagcaccgaccccagccccatgctggccatccctgccatggccaccaacccccagaacgccgccagccgccggacgcccaacaacccggggaacccatccaatcccggcccccccaagacccccgagaatagccttatcgtcaccaaccccagcggcacccagaccaattcagctaagactgccaggaaacccgaccacaccacagtggacatccccccagcctgcccaccccccctcaaccacaccgtcgtacaagtgaacaaaaacgccaacccagacccactgccaaaaaaagaggaagagaagaaggaggaggaggaagacgaccgtggggaagacggccctaagccaatgcctccctatagctccatgttcatcctgtccacgaccaacccccttcgccgcctgtgccattacatcctgaacctgcgctactttgagatgtgcatcctcatggtcattgccatgagcagcatcgccctggccgccgaggaccctgtgcagcccaacgcacctcggaacaacgtgctgcgatactttgactacgtttttacaggcgtctttacctttgagatggtgatcaagatgattgacctggggctcgtcctgcatcagggtgcctacttccgtgacctctggaatattctcgacttcatagtggtcagtggggccctggtagcctttgccttcactggcaatagcaaaggaaaagacatcaacacgattaaatccctccgagtcctccgggtgctacgacctcttaaaaccatcaagcggctgccaaagctcaaggctgtgtttgactgtgtggtgaactcacttaaaaacgtcttcaacatcctcatcgtctacatgctattcatgttcatcttcgccgtggtggctgtgcagctcttcaaggggaaattcttccactgcactgacgagtccaaagagtttgagaaagattgtcgaggcaaatacctcctctacgagaagaatgaggtgaaggcgcgagaccgggagtggaagaagtatgaattccattacgacaatgtgctgtgggctctgctgaccctcttcaccgtgtccacgggagaaggctggccacaggtcctcaagcattcggtggacgccacctttgagaaccagggccccagccccgggtaccgcatggagatgtccattttctacgtcgtctactttgtggtgttccccttcttctttgtcaatatctttgtggccttgatcatcatcaccttccaggagcaaggggacaagatgatggaggaatacagcctggagaaaaatgagagggcctgcattgatttcgccatcagcgccaagccgctgacccgacacatgccgcagaacaagcagagcttccagtaccgcatgtggcagttcgtggtgtctccgcctttcgagtacacgatcatggccatgatcgccctcaacaccatcgtgcttatgatgaagttctatggggcttctgttgcttatgaaaatgccctgcgggtgttcaacatcgtcttcacctccctcttctctctggaatgtgtgctgaaagtcatggcttttgggattctgaattatttccgcgatgcctggaacatcttcgactttgtgactgttctgggcagcatcaccgatatcctcgtgactgagtttgggaataacttcatcaacctgagctttctccgcctcttccgagctgcccggctcatcaaacttctccgtcagggttacaccatccgcattcttctctggacctttgtgcagtccttcaaggccctgccttatgtctgtctgctgatcgccatgctcttcttcatctatgccatcattgggatgcaggtgtttggtaacattggcatcgacgtggaggacgaggacagtgatgaagatgagttccaaatcactgagcacaataacttccggaccttcttccaggccctcatgcttctcttccggagtgccaccggggaagcttggcacaacatcatgctttcctgcctcagcgggaaaccgtgtgataagaactctggcatcctgactcgagagtgtggcaatgaatttgcttatttttactttgtttccttcatcttcctctgctcgtttctgatgctgaatctctttgtcgccgtcatcatggacaactttgagtacctcacccgagactcctccatcctgggcccccaccacctggatgagtacgtgcgtgtctgggccgagtatgaccccgcagcttggggccgcatgccttacctggacatgtatcagatgctgagacacatgtctccgcccctgggtctggggaagaagtgtccggccagagtggcttacaagcggcttctgcggatggacctgcccgtcgcagatgacaacaccgtccacttcaattccaccctcatggctctgatccgcacagccctggacatcaagattgccaagggaggagccgacaaacagcagatggacgctgagctgcggaaggagatgatggcgatttggcccaatctgtcccagaagacgctagacctgctggtcacacctcacaagtccacggacctcaccgtggggaagatctacgcagccatgatgatcatggagtactaccggcagagcaaggccaagaagctgcaggccatgcgcgaggagcaggaccggacacccctcatgttccagcgcatggagcccccgtccccaacgcaggaagggggacctggccagaacgccctcccctccacccagctggacccaggaggagccctgatggctcacgaaagcggcctcaaggagagcccgtcctgggtgacccagcgtgcccaggagatgttccagaagacgggcacatggagtccggaacaaggcccccctaccgacatgcccaacagccagcctaactctcagtccgtggagatgcgagagatgggcagagatggctactccgacagcgagcactacctccccatggaaggccagggccgggctgcctccatgccccgcctccctgcagagaaccagaggagaaggggccggccacgtgggaataacctcagtaccatctcagacaccagccccatgaagcgttcagcctccgtgctgggccccaaggcccgacgcctggacgattactcgctggagcgggtcccgcccgaggagaaccagcggcaccaccagcggcgccgcgaccgcagccaccgcgcctctgagcgctccctgggccgctacaccgatgtggacacaggcttggggacagacctgagcatgaccacccaatccggggacctgccgtcgaaggagcgggaccaggagcggggccggcccaaggatcggaagcatcgacagcaccaccaccaccaccaccaccaccaccatcccccgccccccgacaaggaccgctatgcccaggaacggccggaccacggccgggcacgggctcgggaccagcgctggtcccgctcgcccagcgagggccgagagcacatggcgcaccggcagggcagtagttccgtaagtggaagcccagccccctcaacatctggtaccagcactccgcggcggggccgccgccagctcccccagaccccctccaccccccggccacacgtgtcctattcccctgtgatccgtaaggccggcggctcggggcccccgcagcagcagcagcagcagcagcagcagcagcagcagcaggcggtggccaggccgggccgggcggccaccagcggccctcggaggtacccaggccccacggccgagcctctggccggagatcggccgcccacggggggccacagcagcggccgctcgcccaggatggagaggcgggtcccaggcccggcccggagcgagtcccccagggcctgtcgacacggcggggcccggtggccggcatctggcccgcacgtgtccgaggggcccccgggtccccggcaccatggctactaccggggctccgactacgacgaggccgatggcccgggcagcgggggcggcgaggaggccatggccggggcctacgacgcgccaccccccgtacgacacgcgtcctcgggcgccaccgggcgctcgcccaggactccccgggcctcgggcccggcctgcgcctcgccttctcggcacggccggcgactccccaacggctactacccggcgcacggactggccaggccccgcgggccgggctccaggaagggcctgcacgaaccctacagcgagagtgacgatgattggtgctaa

SEQ ID NO: 41 - protein, 330 aa, Homo sapiens
RGALVGAPRSARASCLRGRRPGRRQPCGRCPDPPRSGPGQAGRARCARDVAAQTALAPRALALGAGAAGPGWRGAVPRRPAARVSRAAQRQEQELVRLHREQECELQPEPAPPRPSGPATAEDPGRRPVLPQRPPEERPPQPPGSTGVIAETGQAGPPAGAGAPGYPKSPPVASPGAPVPSLVSFSAGLTQKPFPSDGGVVLFNKVLVNDGDVYNPSTGVFTAPYDGRYLITATLTPERDAYVEAVLSVSNASVAQLHTAGYRREFLEYHRPPGALHTCGGPGAFHLIVHLKAGDAVNVVVTGGKLAHTDFDEMYSTFSGVFLYPFLSHL

SEQ ID NO: 42 - DNA, 994 bp, Homo sapiens
ccgcggagcactggttggagcgccgcgaagcgcccgagcctcttgccttcgcgggcggcgccctggccgccggcagccttgtggccggtgccccgatccgccgcgctccggacccgggcaggcggggcgcgcccgctgcgcgcgggatgtggcagcccagacggccctggccccgcgtgccctggcgctgggcgctggcgctgctggccctggttggcgcggggctgtgccacgccggcccgcagcccgggtatcccgcgcggcccagcgccaggaacaagaactggtgcgcctacatcgtgaacaagaatgtgagctgcagcccgagcccgccccgccgaggcccagcggccccgcaaccgcagaggaccctgggcgacggcccgtcctgccccagcggccccccgaggagaggccgccccagccgccaggctccaccggggtcatcgcggagacgggccaggccgggccccccgcaggcgcaggcgcaccaggatacccgaagtcacctcctgtagcttccccaggagctccggtgccttctctggtgtctttttctgcggggctcacccagaagcctttccccagtgatgggggcgttgtcctctttaacaaagtgctggtgaacgacggggatgtttacaaccccagcaccggggtcttcacggctccttatgatgggcgctacctgatcacggccaccctcacccccgagagagacgcctacgtggaagcagtgctgtcggtctccaacgccagcgtggcccagctgcataccgctgggtacaggagagagttcctggaataccaccgccctccaggagctttgcatacctgcgggggcccgggggcattccacctcatcgtgcacctgaaggcgggagatgcagtcaacgtcgtggtgactgggggcaagctggctcacacagactttgatgaaatgtactccacatttagtggggttttcttatatcctttcctttcccacctctaa

SEQ ID NO: 43 - protein, 97 aa, Homo sapiens
MKEPGPNFVTVRKGLHSFKMAFVKHLLLFLSPRLECSGSITDHCSLHLPVQEILMSQPPEQLGLQTNLGNQESSGMMKLFMPRPKVLAQYESIQFMP

SEQ ID NO: 44 - DNA, 294 bp, Homo sapiens
atgaaggagcctggacccaactttgttactgtgagaaagggtcttcattcattcaagatggcatttgttaagcacctacttctctttctgtcacctaggctggagtgcagtggttcaatcacggatcactgcagcctccacctcccagttcaagaaattctcatgtctcagcctcctgagcagctaggattacagacaaaccttggaaatcaagaaagttctggaatgatgaagctgttcatgccaagaccgaaagtgctggcccagtatgagtccattcagttcatgccgtga

SEQ ID NO: 45 - protein, 47 aa, Homo sapiens
MHNIMMVKLKKKKSTTNLGNQESSGMMKLFMPRPKVLAQYESIQFMP

SEQ ID NO: 46 - protein, 43 aa, Homo sapiens
MSQPPEQLGLQTNLGNQESSGMMKLFMPRPKVLAQYESIQFMP

SEQ ID NO: 47 - protein, 70 aa, Homo sapiens
MYSYWRLECSGSITDHCSLHLPVQEILMSQPPEQLGLQTNLGNQESSGMMKLFMPRPKVLAQYESIQFMP

SEQ ID NO: 48 - protein, 91 aa, Homo sapiens
MKEPGPNFVTVRKGLHSFKMAFVKHLLLECSGSITDHCSLHLPVQEILMSQPPEQLGLQTNLGNQESSGMMKLFMPRPKVLAQYESIQFMP

SEQ ID NO: 49 - protein, 37 aa, Homo sapiens
MKEPGPNFVTVRKGLHSFKMAFVKHLLQTLEIKKVLE

SEQ ID NO: 50 - protein, 1079 aa, Homo sapiens
MVCSAAPLLLLATTLPLLGSPVAQASQPVSETGVRPREGLQRRQWGPLIGRDKAWNERIDRPFPACPIPLSSSFGRWPKGQTMWAQTSTLTLTEEELGQSQAGGESGSGQLLDQENGAGESALVSVYVHLDFPDKTWPPELSRTLTLPAASASSSPRPLLTGLRLTTECNVNHKGNFYCACLSGYQWNTSICLHYPPCQSLHNHQPCGCLVFSHPEPGYCQLLPPGSPVTCLPAVPGILNLNSQLQMPGDTLSLTLHLSQEATNLSWFLRHPGSPSPILLQPGTQVSVTSSHGQAALSVSNMSHHWAGEYMSCFEAQGFKWNLYEVVRVPLKATDVARLPYQLSISCATSPGFQLSCCIPSTNLAYTAAWSPGEGSKASSFNESGSQCFVLAVQRCPMADTTYACDLQSLGLAPLRVPISITIIQDGDITCPEDASVLTWNVTKAGHVAQAPCPESKRGIVRRLCGADGVWGPVHSSCTDARLLALFTRTKLLQAGQGSPAEEVPQILAQLPGQAAEASSPSDLLTLLSTMKYVAKVVAEARIQLDRRALKNLLIATDKVLDMDTRSLWTLAQARKPWAGSTLLLAVETLACSLCPQDHPFAFSLPNVLLQSQLFGPTFPADYSISFPTRPPLQAQIPRHSLAPLVRNGTEISITSLVLRKLDHLLPSNYGQGLGDSLYATPGLVLVISIMAGDRAFSQGEVIMDFGNTDGSPHCVFWDHSLFQGRGGWSKEGCQAQVASASPTAQCLCQHLTAFSVLMSPHTVPEEPALALLTQVGLGASILALLVCLGVYWLVWRVVVRNKISYFRHAALLNMVFCLLAADTCFLGAPFLSPGPRSPLCLAAAFLCHFLYLATFFWMLAQALVLAHQLLFVFHQLAKHRVLPLMVLLGYLCPLGLAGVTLGLYLPQGQYLREGECWLDGKGGALYTFVGPVLAIIGVNGLVLAMAMLKLLRPSLSEGPPAEKRQALLGVIKALLILTPIFGLTWGLGLATLLEEVSTVPHYIFTILNTLQGVFILLFGCLMDRKIQEALRKRFCRAQAPSSTISLVSCCLQILSCASKSMSEGIPWPSSEDMGTARS

SEQ ID NO: 51 - DNA, 3240 bp, Homo sapiens
atggtctgttcggctgccccactgctgctcctggccacaactcttcccctgctggggtcaccagttgcccaagcatcccaacctgtaagtgagactggggtgagacccagggaaggtctgcagaggcgacaatggggacccctgattgggagagacaaagcatggaatgaaaggatagacagacccttccctgcctgccccatccccctatcttctagctttggccgatggcccaagggccagacaatgtgggcccagacctccaccctcaccctgacagaggaggagttgggacagagtcaggctggaggggaatctggatctgggcagctcctggaccaagagaatggagcaggggaatcagcgctggtctccgtctatgtacatctggactttccagataagacctggccccctgaactctccaggacactgactctccctgctgcctcagcttcctcttccccaaggcctcttctcactggcctcagactcacaacagagtgtaatgtcaaccacaaggggaatttctattgtgcttgcctctctggctaccagtggaacaccagcatctgcctccattaccctccttgtcaaagcctccacaaccaccagccttgtggctgccttgtcttcagccatcccgaacccgggtactgccagttgctgccacctgggtcccctgtcacctgcctccctgcagtccccgggatcctcaacctgaactcccagctgcagatgcctggtgacacgctgagcctgactctccatctgagccaggaggccaccaacctgagctggttcctgaggcacccagggagccccagtcccatcctcctgcagccagggacacaggtgtctgtgacttccagccacggccaggctgccctcagcgtctccaacatgtcccatcactgggcaggtgagtacatgagctgcttcgaggcccagggcttcaagtggaacctgtatgaggtggtgagggtgcccttgaaggcgacagatgtggctcgacttccataccagctgtccatctcctgtgccacctcccctggcttccagctgagctgctgcatccccagcacaaacctggcctacaccgcggcctggagccctggagagggcagcaaagcttcctccttcaacgagtcaggctctcagtgctttgtgctggctgttcagcgctgcccgatggctgacaccacgtacgcttgtgacctgcagagcctgggcctggctccactcagggtccccatctccatcaccatcatccaggatggagacatcacctgccctgaggacgcctcggtgctcacctggaatgtcaccaaggctggccacgtggcacaggccccatgtcctgagagcaagaggggcatagtgaggaggctctgtggggctgacggagtctgggggccggtccacagcagctgcacagatgcgaggctcctggccttgttcactagaaccaagctgctgcaggcaggccagggcagtcctgctgaggaggtgccacagatcctggcacagctgccagggcaggcggcagaggcaagttcaccctccgacttactgaccctgctgagcaccatgaaatacgtggccaaggtggtggcagaggccagaatacagcttgaccgcagagccctgaagaatctcctgattgccacagacaaggtcctagatatggacaccaggtctctgtggaccctggcccaagcccggaagccctgggcaggctcgactctcctgctggctgtggagaccctggcatgcagcctgtgcccacaggaccaccccttcgccttcagcttacccaatgtgctgctgcagagccagctgtttggacccacgtttcctgctgactacagcatctccttccctactcggcccccactgcaggctcagattcccaggcactcactggccccattggtccgtaatggaactgaaataagtattactagcctggtgctgcgaaaactggaccaccttctgccctcaaactatggacaagggctgggggattccctctatgccactcctggcctggtccttgtcatttccatcatggcaggtgaccgggccttcagccagggagaggtcatcatggactttgggaacacagatggttcccctcactgtgtcttctgggatcacagtctcttccagggcagggggggttggtccaaagaagggtgccaggcacaggtggccagtgccagccccactgctcagtgcctctgccagcacctcactgccttctccgtcctcatgtccccacacactgttccggaagaacccgctctggcgctgctgactcaagtgggcttgggagcttccatactggcgctgcttgtgtgcctgggtgtgtactggctggtgtggagagtcgtggtgcggaacaagatctcctatttccgccacgccgccctgctcaacatggtgttctgcttgctggccgcagacacttgcttcctgggcgccccattcctctctccagggccccgaagcccgctctgccttgctgccgccttcctctgtcatttcctctacctggccacctttttctggatgctggcgcaggccctggtgttggcccaccagctgctctttgtctttcaccagctggcaaagcaccgagttctccccctcatggtgctcctgggctacctgtgcccactggggttggcaggtgtcaccctggggctctacctacctcaagggcaatacctgagggagggggaatgctggttggatgggaagggaggggcgttatacaccttcgtggggccagtgctggccatcataggcgtgaatgggctggtactagccatggccatgctgaagttgctgagaccttcgctgtcagagggacccccagcagagaagcgccaagctctgctgggggtgatcaaagccctgctcattcttacacccatctttggcctcacctgggggctgggcctggccactctgttagaggaagtctccacggtccctcattacatcttcaccattctcaacaccctccagggcgtcttcatcctattgtttggttgcctcatggacaggaagatacaagaagctttgcgcaaacgcttctgccgcgcccaagcccccagctccaccatctccctggtgagttgctgccttcagatcctcagctgtgcatccaagagcatgtcagaaggcattccatggccctcctcagaggacatgggcacagccagaagctaa

SEQ ID NO: 52 - protein, 321 aa, Homo sapiens
MAMATKGGTVKAASGFNAMEDAQTLRKAMKGLGTDEDAIISVLAYRNTAQRQEIRTAYKSTIGRDLIDDLKSELSGNFEQVIVGMMTPTVLYDVQELRRAMKGAGTDEGCLIEILASRTPEEIRRISQTYQQQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAEKLYKSMKGLGTDDNTLIRVMVSRAEIDMLDIRAHFKRLYGKSLYSFIKGDTSGDYRKVLLVLCGGDD

SEQ ID NO: 53 - DNA, 963 bp, Homo sapiens
atggccatggcaaccaaaggaggtactgtcaaagctgcttcaggattcaatgccatggaagatgcccagaccctgaggaaggccatgaaagggctcggcaccgatgaagacgccattattagcgtccttgcctaccgcaacaccgcccagcgccaggagatcaggacagcctacaagagcaccatcggcagggacttgatagacgacctgaagtcagaactgagtggcaacttcgagcaggtgattgtggggatgatgacgcccacggtgctgtatgacgtgcaagagctgcgaagggccatgaagggagccggcactgatgagggctgcctaattgagatcctggcctcccggacccctgaggagatccggcgcataagccaaacctaccagcagcaatatggacggagccttgaagatgacattcgctctgacacatcgttcatgttccagcgagtgctggtgtctctgtcagctggtgggagggatgaaggaaattatctggacgatgctctcgtgagacaggatgcccaggacctgtatgaggctggagagaagaaatgggggacagatgaggtgaaatttctaactgttctctgttcccggaaccgaaatcacctgttgcatgtgtttgatgaatacaaaaggatatcacagaaggatattgaacagagtattaaatctgaaacatctggtagctttgaagatgctctgctggctatagtaaagtgcatgaggaacaaatctgcatattttgctgaaaagctctataaatcgatgaagggcttgggcaccgatgataacaccctcatcagagtgatggtttctcgagcagaaattgacatgttggatatccgggcacacttcaagagactctatggaaagtctctgtactcgttcatcaagggtgacacatctggagactacaggaaagtactgcttgttctctgtggaggagatgat

SEQ ID NO: 54 - protein, 318 aa, Homo sapiens
ATKGGTVKAASGFNAMEDAQTLRKAMKGLGTDEDAIISVLAYRNTAQRQEIRTAYKSTIGRDLIDDLKSELSGNFEQVIVGMMTPTVLYDVQELRRAMKGAGTDEGCLIEILASRTPEEIRRISQTYQQQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAEKLYKSMKGLGTDDNTLIRVMVSRAEIDMLDIRAHFKRLYGKSLYSFIKGDTSGDYRKVLLVLCGGDD

SEQ ID NO: 55 - DNA, 966 bp, Homo sapiens
atggccatggcaaccaaaggaggtactgtcaaagctgcttcaggattcaatgccatggaagatgcccagaccctgaggaaggccatgaaagggctcggcaccgatgaagacgccattattagcgtccttgcctaccgcaacaccgcccagcgccaggagatcaggacagcctacaagagcaccatcggcagggacttgatagacgacctgaagtcagaactgagtggcaacttcgagcaggtgattgtggggatgatgacgcccacggtgctgtatgacgtgcaagagctgcgaagggccatgaagggagccggcactgatgagggctgcctaattgagatcctggcctcccggacccctgaggagatccggcgcataagccaaacctaccagcagcaatatggacggagccttgaagatgacattcgctctgacacatcgttcatgttccagcgagtgctggtgtctctgtcagctggtgggagggatgaaggaaattatctggacgatgctctcgtgagacaggatgcccaggacctgtatgaggctggagagaagaaatgggggacagatgaggtgaaatttctaactgttctctgttcccggaaccgaaatcacctgttgcatgtgtttgatgaatacaaaaggatatcacagaaggatattgaacagagtattaaatctgaaacatctggtagctttgaagatgctctgctggctatagtaaagtgcatgaggaacaaatctgcatattttgctgaaaagctctataaatcgatgaagggcttgggcaccgatgataacaccctcatcagagtgatggtttctcgagcagaaattgacatgttggatatccgggcacacttcaagagactctatggaaagtctctgtactcgttcatcaagggtgacacatctggagactacaggaaagtactgcttgttctctgtggaggagatgattaa

SEQ ID NO: 56 - protein, 1318 aa, Homo sapiens
MLRTSGLALLALVSAVGPSQASGFTEKGLSLLGYQLCSHRVTHTVQKVEAVQTSYTSYVSCGGWIPWRRCPKMVYRTQYLVVEVPESRNVTDCCEGYEQLGLYCVLPLNQSGQFTSRPGACPAEGPEPSTSPCSLDIDCPGLEKCCPWSGGRYCMAPAPQAPERDPVGSWYNVTILVKMDFKELQQVDPRLLNHMRLLHSLVTSALQPMASTVHHLHSAPGNASTTVSRLLLGLPRPLPVADVSTLLGDIAKRVYEVISVQVQDVNECFYEELNACSGRELCANLEGSYWCVCHQEAPATSPRKLNLEWEDCPPVSDYVVLNVTSDSFQVSWRLNSTQNHTFHVRVYRGMELLRSARTQSQALAVAGLEAGVLYRVKTSYQGCGADVSTTLTIKTNAQVFEVTIKIVNHNLTEKLLNRSSVEYQDFSRQLLHEVESSFPPVVSDLYRSGKLRMQIVSLQAGSVVVRLKLTVQDPGFPMGISTLAPILQPLLASTVFQIDRQGTRVQDWDECVDSAEHDCSPAAWCINLEGSYTCQCRTTRDATPSRAGRACEGDLVSPMGGGLSAATGVTVPGLGTGTAALGLENFTLSPSPGYPQGTPAAGQAWTPEPSPRRGGSNVVGYDRNNTGKGVEQELQGNSIMEPPSWPSPTEDPTGHFLWHATRSTRETLLNPTWLRNEDSGPSGSVDLPLTSTLTALKTPACVPVSIGRIMVSNVTSTGFHLAWEADLAMDSTFQLTLTSMWSPAVVLETWNTSVTLSGLEPGVLHLVEIMAKACGKEGARAHLKVRTAARKLIGKVRIKNVRYSESFRNASSQEYRDFLELFFRMVRGSLPATMCQHMDAGGVRMEVVSVTNGSIVVEFHLLIIADVDVQEVSAAFLTAFQTVPLLEVIRGDTFIQDYDECERKEDDCVPGTSCRNTLGSFTCSCEGGAPDFPVEYSERPCEGDSPGNETWATSPERPLTTAGTKAAFVQGTSPTPQGLPQRLNLTGAVRVLCEIEKVVVAIQKRFLQQESIPESSLYLSHPSCNVSHSNGTHVLLEAGWSECGTLMQSNMTNTVVRTTLRNDLSQEGIIHHLKILSPIYCAFQNDLLTSSGFTLEWGVYTIIEDLHGAGNFVTEMQLFIGDSPIPQNYSVSASDDVRIEVGLYRQKSNLKVVLTECWATPSSNARDPITFSFINNSCPVPNTYTNVIENGNSNKAQFKLRIFSFINDSIVYLHCKLRVCMESPGATCKINCNNFRLLQNSETSATHQMSWGPLIRSEGEPPHAEAGLGAGYVVLIVVAIFVLVAGTATLLIVRYQRMNGRYNFKIQSNNFSYQVFYE

SEQ ID NO: 57 - DNA, 3957 bp, Homo sapiens
atgctcaggacctcggggctggcactgctggctctggtcagtgctgtgggcccaagccaggccagcggcttcacagaaaaaggcctctccctgttgggctaccagctatgcagccaccgtgtgacccacactgtacagaaggtggaggccgtgcagacgtcctacacgtcctatgtgtcctgcggcggctggatcccctggaggcggtgccctaagatggtttaccggacacagtacctggtagtggaggtccccgagtccaggaacgtgactgactgctgtgagggctatgaacagctcggcctctactgtgtcttgcccctgaatcagtccgggcagttcacgtcaagacccggggcctgccccgcagaggggcctgaaccatccacctccccctgcagcttggacatcgactgtcctggacttgagaagtgctgcccctggtcaggggggcgctactgcatggcccctgcaccccaagctccagagagggaccctgtgggctcctggtacaacgtcaccatactggtgaaaatggacttcaaggaactccagcaagtggaccccaggctcctgaaccacatgcgccttctgcattccttggtcaccagcgccctgcaaccaatggcctccaccgtccaccacctgcactcagcccctgggaacgcctccaccacagtgtcgcggctgctactgggcctgccacggccactgcctgtggctgacgtctccaccctgctgggtgacattgcgaagcgtgtctatgaagtgatcagcgtccaggtgcaagatgtcaatgagtgtttctatgaggagctcaatgcctgctctggaagggaactgtgcgcaaacctggagggctcgtactggtgcgtctgtcaccaggaagctccagcgacgtctccacggaagctgaacctggagtgggaagattgtcctccagtcagtgactacgtggtcctcaacgtcaccagtgacagttttcaagtatcctggcgtttaaattctacacagaaccacactttccatgtccgggtttaccggggtatggagttgctcaggagcgccaggacacagagccaggcactggcagtggctgggctggaggctggagtgctgtacagggtgaagaccagctaccaggggtgcggggccgacgtctccaccacgctgaccatcaaaaccaatgcccaggtatttgaagtcacaataaagattgtaaaccacaacctgacggagaagttactcaaccgcagcagcgtggagtaccaggacttttctagacaactgcttcacgaggtcgagagctccttcccaccagtggtgtctgacttgtaccgaagtgggaagctgagaatgcagatcgtgtctctccaggcgggaagtgtggtcgtgaggctcaagctcaccgtgcaggaccccgggtttcccatgggcatctccacgctggcccccatactccagcccctgttggcaagcacagtgttccagattgaccggcaggggacacgcgtgcaagactgggacgagtgtgtggacagcgcggaacacgactgctcaccggctgcctggtgcatcaacctggagggctcctacacctgccagtgccgtaccaccagggacgccaccccctcccgcgcaggccgggcctgtgagggtgacctggtgagccccacgggcggtggactgtctgcggcaacaggggtaacggtcccaggtcttggcacgggaacagcagccctcggcctagagaacttcaccttgtcacccagtcctgggtaccctcagggcaccccggcagcaggccaggcctggaccccagagccctcacccagaagagggggcagcaatgtggtcgggtatgacaggaacaacacaggaaaaggcgtggagcaggagctacagggaaactccaccatggagccaccctcctggccttcccctactgaggaccccaccggccacttcctgtggcatgccacccgttccacccgggaaacacttctgaatcccacgtggctgcgaaatgaggacagtggaccctccggttctgtagacctgccattgacctccaccctcacagctctgaagacccccgcctgtgttcctgtctccattgggaggatcatggtctccaatgtgaccagcaccggcttccacctggcatgggaggcggatcttgctatggactccaccttccagctcactctgacttccatgtggagccctgctgtggtcctagagacctggaacacgagtgtgacactgtcggggctggagcctggggtcttgcacctggttgagatcatggccaaagcatgtgggaaagaaggtgccagagctcatctgaaagtgaggacagcagcccggaagctcattggaaaggtcagaatcaaaaatgtcaggtactcagaatcctttcgcaacgcaagcagccaggagtatcgagatttcctagaactattcttcaggatggtgcggggctccctgccagccaccatgtgtcagcacatggacgctggtggggtcaggatggaagtcgtcagcgtcaccaacggcagcatcgtggtggagtttcacttgctgataatcgcagatgtggatgtccaggaggtgtcagctgcatttctcaccgccttccagaccgtgcctctgctggaggtgatcagaggcgacaccttcatacaggattacgatgagtgtgaaaggaaggaggacgactgtgtgccggggacatcctgtcgaaacaccctcgggtctttcacttgtagctgcgagggaggagcccccgacttccctgtggaatattctgagagaccctgtgaaggtgactctcctggcaatgaaacctgggccaccagcccagagaggcctctcaccacagcagggaccaaggctgcctttgtgcaaggcaccagccccaccccccaaggcctgccccagcggctgaacctgaccggagcagtcagggtgctctgtgagatcgagaaggtggttgtcgccatccagaagcgcttcctgcagcaggaatccatccccgagtcctcgttgtacctcagccacccctcctgcaacgtgagccacagcaatggcacacacgtgctcctggaggccggctggagcgagtgtgggaccctcatgcagagcaacatgacgaacaccgtggtgaggaccacgctgaggaacgacctgtcccaggagggcatcatccaccacctgaagatcctgagccccatctactgcgccttccagaatgacctgctgacatcctccggcttcaccctggagtggggggtttacaccatcatcgaggacctccacggcgctgggaattttgttaccgaaatgcagttgtttatcggagactctcccatacctcagaattatagcgtgtctgccagtgacgatgtcaggatcgaagtggggctctacaggcagaaaagcaacctcaaggtggtcctgacggagtgctgggcaaccccgtctagcaacgcccgggaccccatcaccttcagcttcattaacaacagctgccccgtgcccaacacatacaccaacgtgattgagaacggcaactccaataaggcccagttcaagctgaggatcttttcctttatcaacgactccatcgtctacctgcactgcaaactccgcgtctgcatggaatcccccggagccacgtgcaaaatcaattgcaataactttcggttgctgcaaaatagtgaaacctctgccacacaccagatgtcctggggacccctcatccggtctgaaggtgagcctccacatgcagaagcaggcctgggtgccggttatgtggtccttattgtggtggccatcttcgtgctggtggcgggaacagccacccttctgatcgtgcgctaccagagaatgaatgggagatacaactttaaaatccagtcccacaacttcagctaccaggtgttctacgaatag

SEQ ID NO: 58 - protein, 1446 aa, Homo sapiens
MLRTSGLALLALVSAVGPSQASGFTEKGLSLLGYQLCSHRVTHTVQKVEAVQTSYTSYVSCGGWIPWRRCPKMVYRTQYLVVEVPESRNVTDCCEGYEQLGLYCVLPLNQSGQFTSRPGACPAEGPEPSTSPCSLDIDCPGLEKCCPWSGGRYCMAPAPQAPERDPVGSWYNVTILVKMDFKELQQVDPRLLNHMRLLHSLVTSALQPMASTVHHLHSAPGNASTTVSRLLLGLPRPLPVADVSTLLGDIAKRVYEVISVQVQDVNECFYEELNACSGRELCANLEGSYWCVCHQEAPATSPRKLNLEWEDCPPVSDYVVLNVTSDSFQVSWRLNSTQNHTFHVRVYRGMELLRSARTQSQALAVAGLEAGVLYRVKTSYQGCGADVSTTLTIKTNAQVFEVTIKIVNHNLTEKLLNRSSVEYQDFSRQLLHEVESSFPPVVSDLYRSGKLRMQIVSLQAGSVVVRLKLTVQDPGFPMGISTLAPILQPLLASTVFQIDRQGTRVQDWDECVDSAEHDCSPAAWCINLEGSYTCQCRTTRDATPSRAGRACEGDLVSPMGGGLSAATGVTVPGLGTGTAALGLENFTLSPSPGYPQGTPAAGQAWTPEPSPRRGGSNVVGYDRNNTGKGVEQEVPSTAPGLGMDQGSPSQVNPSQGSPSQGSLRQESTSQASPSQRSTSQGSPSQVNPSQGSTSHANSSQGSPSQGSPSQESPSQGSTSQASPSHRNTIGVIGTTSSPKATGSTHSFPPGATDGPLALPGQLQGNSIMEPPSWPSPTEDPTGHFLWHATRSTRETLLNPTWLRNEDSGPSGSVDLPLTSTLTALKTPACVPVSIGRIMVSNVTSTGFHLAWEADLAMDSTFQLTLTSMWSPAVVLETWNTSVTLSGLEPGVLHLVEIMAKACGKEGARAHLKVRTAARKLIGKVRIKNVRYSESFRNASSQEYRDFLELFFRMVRGSLPATMCQHMDAGGVRMEVVSVTNGSIVVEFHLLIIADVDVQEVSAAFLTAFQTVPLLEVIRGDTFIQDYDECERKEDDCVPGTSCRNTLGSFTCSCEGGAPDFPVEYSERPCEGDSPGNETWATSPERPLTTAGTKAAFVQGTSPTPQGLPQRLNLTGAVRVLCEIEKVVVAIQKRFLQQESIPESSLYLSHPSCNVSHSNGTHVLLEAGWSECGTLMQSNMTNTVVRTTLRNDLSQEGIIHHLKILSPIYCAFQNDLLTSSGFTLEWGVYTIIEDLHGAGNFVTEMQLFIGDSPIPQNYSVSASDDVRIEVGLYRQKSNLKVVLTECWATPSSNARDPITFSFINNSCPVPNTYTNVIENGNSNKAQFKLRIFSFINDSIVYLHCKLRVCMESPGATCKINCNNFRLLQNSETSATHQMSWGPLIRSEGEPPHAEAGLGAGYVVLIVVAIFVLVAGTATLLIVRYQRMNGRYNFKIQSNNFSYQVFYE

SEQ ID NO: 59 - protein, 1246 aa, Homo sapiens
MVYRTQYLVVEVPESRNVTDCCEGYEQLGLYCVLPLNQSGQFTSRPGACPAEGPEPSTSPCSLDIDCPGLEKCCPWSGGRYCMAPAPQAPERDPVGSWYNVTILVKMDFKELQQVDPRLLNHMRLLHSLVTSALQPMASTVHHLHSAPGNASTTVSRLLLGLPRPLPVADVSTLLGDIAKRVYEVISVQVQDVNECFYEELNACSGRELCANLEGSYWCVCHQEAPATSPRKLNLEWEDCPPVSDYVVLNVTSDSFQVSWRLNSTQNHTFHVRVYRGMELLRSARTQSQALAVAGLEAGVLYRVKTSYQGCGADVSTTLTIKTNAQVFEVTIKIVNHNLTEKLLNRSSVEYQDFSRQLLHEVESSFPPVVSDLYRSGKLRMQIVSLQAGSVVVRLKLTVQDPGFPMGISTLAPILQPLLASTVFQIDRQGTRVQDWDECVDSAEHDCSPAAWCINLEGSYTCQCRTTRDATPSRAGRACEGDLVSPMGGGLSAATGVTVPGLGTGTAALGLENFTLSPSPGYPQGTPAAGQAWTPEPSPRRGGSNVVGYDRNNTGKGVEQELQGNSIMEPPSWPSPTEDPTGHFLWHATRSTRETLLNPTWLRNEDSGPSGSVDLPLTSTLTALKTPACVPVSIGRIMVSNVTSTGFHLAWEADLAMDSTFQLTLTSMWSPAVVLETWNTSVTLSGLEPGVLHLVEIMAKACGKEGARAHLKVRTAARKLIGKVRIKNVRYSESFRNASSQEYRDFLELFFRMVRGSLPATMCQHMDAGGVRMEVVSVTNGSIVVEFHLLIIADVDVQEVSAAFLTAFQTVPLLEVIRGDTFIQDYDECERKEDDCVPGTSCRNTLGSFTCSCEGGAPDFPVEYSERPCEGDSPGNETWATSPERPLTTAGTKAAFVQGTSPTPQGLPQRLNLTGAVRVLCEIEKVVVAIQKRFLQQESIPESSLYLSHPSCNVSHSNGTHVLLEAGWSECGTLMQSNMTNTVVRTTLRNDLSQEGIIHHLKILSPIYCAFQNDLLTSSGFTLEWGVYTIIEDLHGAGNFVTEMQLFIGDSPIPQNYSVSASDDVRIEVGLYRQKSNLKVVLTECWATPSSNARDPITFSFINNSCPVPNTYTNVIENGNSNKAQFKLRIFSFINDSIVYLHCKLRVCMESPGATCKINCNNFRLLQNSETSATHQMSWGPLIRSEGEPPHAEAGLGAGYVVLIVVAIFVLVAGTATLLIVRYQRMNGRYNFKIQSNNFSYQVFYE

SEQ ID NO: 60 - protein, 1374 aa, Homo sapiens
MVYRTQYLVVEVPESRNVTDCCEGYEQLGLYCVLPLNQSGQFTSRPGACPAEGPEPSTSPCSLDIDCPGLEKCCPWSGGRYCMAPAPQAPERDPVGSWYNVTILVKMDFKELQQVDPRLLNHMRLLHSLVTSALQPMASTVHHLHSAPGNASTTVSRLLLGLPRPLPVADVSTLLGDIAKRVYEVISVQVQDVNECFYEELNACSGRELCANLEGSYWCVCHQEAPATSPRKLNLEWEDCPPVSDYVVLNVTSDSFQVSWRLNSTQNHTFHVRVYRGMELLRSARTQSQALAVAGLEAGVLYRVKTSYQGCGADVSTTLTIKTNAQVFEVTIKIVNHNLTEKLLNRSSVEYQDFSRQLLHEVESSFPPVVSDLYRSGKLRMQIVSLQAGSVVVRLKLTVQDPGFPMGISTLAPILQPLLASTVFQIDRQGTRVQDWDECVDSAEHDCSPAAWCINLEGSYTCQCRTTRDATPSRAGRACEGDLVSPMGGGLSAATGVTVPGLGTGTAALGLENFTLSPSPGYPQGTPAAGQAWTPEPSPRRGGSNVVGYDRNNTGKGVEQEVPSTAPGLGMDQGSPSQVNPSQGSPSQGSLRQESTSQASPSQRSTSQGSPSQVNPSQGSTSHANSSQGSPSQGSPSQESPSQGSTSQASPSHRNTIGVIGTTSSPKATGSTHSFPPGATDGPLALPGQLQGNSIMEPPSWPSPTEDPTGHFLWHATRSTRETLLNPTWLRNEDSGPSGSVDLPLTSTLTALKTPACVPVSIGRIMVSNVTSTGFHLAWEADLAMDSTFQLTLTSMWSPAVVLETWNTSVTLSGLEPGVLHLVEIMAKACGKEGARAHLKVRTAARKLIGKVRIKNVRYSESFRNASSQEYRDFLELFFRMVRGSLPATMCQHMDAGGVRMEVVSVTNGSIVVEFHLLIIADVDVQEVSAAFLTAFQTVPLLEVIRGDTFIQDYDECERKEDDCVPGTSCRNTLGSFTCSCEGGAPDFPVEYSERPCEGDSPGNETWATSPERPLTTAGTKAAFVQGTSPTPQGLPQRLNLTGAVRVLCEIEKVVVAIQKRFLQQESIPESSLYLSHPSCNVSHSNGTHVLLEAGWSECGTLMQSNMTNTVVRTTLRNDLSQEGIIHHLKILSPIYCAFQNDLLTSSGFTLEWGVYTIIEDLHGAGNFVTEMQLFIGDSPIPQNYSVSASDDVRIEVGLYRQKSNLKVVLTECWATPSSNARDPITFSFINNSCPVPNTYTNVIENGNSNKAQFKLRIFSFINDSIVYLHCKLRVCMESPGATCKINCNNFRLLQNSETSATHQMSWGPLIRSEGEPPHAEAGLGAGYVVLIVVAIFVLVAGTATLLIVRYQRMNGRYNFKIQSNNFSYQVFYE

SEQ ID NO: 61 - protein, 866 aa, Homo sapiens
MEGAGPRGAGPARRRGAGGPPSPLLPSLLLLLLLWMLPDTVAPQELNPRGRNVCRAPGSQVPTCCAGWRQQGDECGIAVCEGNSTCSENEVCVRPGECRCRHGYFGANCDTKCPRQFWGPDCKELCSCHPHGQCEDVTGQCTCHARRWGARCEHACQCQHGTCHPRSGACRCEPGWWGAQCASACYCSATSRCDPQTGACLCHAGWWGRSCNNQCACNSSPCEQQSGRCQCRERTFGARCDRYCQCFRGRCHPVDGTCACEPGYRGKYCREPCPAGFYGLGCRRRCGQCKGQQPCTVAEGRCLTCEPGWNGTKCDQPCATGFYGEGCSHRCPPCRDGHACNHVTGKCTRCNAGWIGDRCETKCSNGTYGEDCAFVCADCGSGHCDFQSGRCLCSPGVHGPHCNVTCPPGLHGADCAQACSCHEDTCDPVTGACHLETNQRKGVMGAGALLVLLVCLLLSLLGCCCACRGKDPTRRELSLGRKKAPHRLCGRFSRISMKLPRIPLRRQKLPKVVVAHHDLDNTLNCSFLEPPSGLEQPSPSWSSRASFSSFDTTDEGPVYCVPHEEAPAESRDPEVPTVPAEAPAPSPVPLTTPASAEEAIPLPASSDSERSASSVEGPGGALYARVARREARPARARGEIGGLSLSPSPERRKPPPPDPATKPKVSWIHGKHSAAAAGRAPSPPPPGSEAAPSPSKRKRTPSDKSAHTVEHGSPRTRDPTPRPPGLPEEATALAAPSPPRARARGRGPGLLEPTDAGGPPRSAPEAASMLAAELRGKTRSLGRAEVALGAQGPREKPAPPQKAKRSVPPASPARAPPATETPGPEKAATDLPAPETPRKKTPIQKPPRKKSREAAGELGRAGAPTL

SEQ ID NO: 62 - DNA, 2601 bp, Homo sapiens
atggagggcgcagggccccggggggccgggccggcgcggcgccggggagccggggggccgccgtcaccgctgctgccgtcgctgctgctgctgctgctgctctggatgctgccggacaccgtggcgcctcaggaactgaaccctcgcggccgcaacgtgtgccgtgctcccggctcccaggtgcccacgtgctgcgctggctggaggcagcaaggggacgagtgtgggattgcggtgtgcgaaggcaactccacgtgctcagagaacgaggtgtgcgtgaggcctggcgagtgccgctgccgccacggctacttcggtgccaactgcgacaccaagtgcccgcgccagttctggggccccgactgcaaggagctgtgtagctgccacccacacgggcagtgcgaggacgtgacaggccagtgtacttgtcacgcgcggcgctggggcgcgcgctgcgagcatgcgtgccagtgccagcacggcacgtgccacccgcggagcggcgcgtgccgctgtgagcccggctggtggggcgcgcagtgcgccagcgcgtgctactgcagcgccacgtcgcgctgcgacccacagaccggcgcctgcctgtgccacgcaggctggtggggccgcagctgcaacaaccagtgcgcctgcaactcgtctccctgcgagcagcagagcggccgctgtcagtgccgcgagcgtacgttcggcgcgcgctgcgatcgctactgccagtgcttccgcggccgctgccaccctgtggacggcacgtgtgcctgcgagccgggctaccgcggcaagtactgtcgcgagccgtgccccgccggcttctacggcttgggctgtcgccgccggtgtggccagtgcaagggccagcagccgtgcacggtggccgagggccgctgcttgacgtgcgagcccggctggaacggaaccaagtgcgaccagccttgcgccaccggtttctatggcgagggctgcagccaccgctgtccgccatgccgcgacgggcatgcctgtaaccatgtcaccggcaagtgtacgcgctgcaacgcgggctggatcggcgaccggtgcgagaccaagtgtagcaatggcacttacggcgaggactgcgccttcgtgtgcgccgactgcggcagcggacactgcgacttccagtcggggcgctgcctgtgcagccctggcgtccacgggccccactgtaacgtgacgtgcccgcccggactccacggcgcggactgtgctcaggcctgcagctgccacgaggacacgtgcgacccggtcactggtgcctgccacctagaaaccaaccagcgcaagggcgtgatgggcgcgggcgcgctgctcgtcctgctcgtctgcctgctgctctcgctgctcggctgctgctgcgcttgccgcggcaaggaccctacgcgccgggagctttcgcttgggaggaagaaggcgccgcaccgactatgcgggcgcttcagtcgcatcagcatgaagctgccccggatcccgctccggaggcagaaactacccaaagtcgtagtggcccaccacgacctggataacacactcaactgcagcttcctggagccaccctcagggctggagcagccctcaccatcctggtcctctcgggcctccttctcctcgtttgacaccactgatgaaggccctgtgtactgtgtaccccatgaggaggcaccagcggagagccgggaccccgaagtccccactgtccctgccgaggcgccggcgccgtcccctgtgcccttgaccacgccagcctccgccgaggaggcgatacccctccccgcgtcctccgacagcgagcggtcggcgtccagcgtggaggggcccggaggggctctgtacgcgcgcgtggcccgacgcgaggcccggccggcccgggcccggggcgagattgggggcctgtcgctgtcgccatcgcccgagcgcaggaaaccgccgccacctgaccccgccaccaagcctaaggtgtcctggatccacggcaagcacagcgccgctgcagctggccgtgcgccctcaccaccgccgccaggctccgaggccgcgcccagccccagcaagaggaaacggacgcccagcgacaaatcggcgcatacggtcgaacacggcagcccccggacccgcgacccaacgccgcggccccccgggctgcccgaggaggcgacagccctcgctgcgccctcgccgcccagggcccgagcgcggggccgcggccccggcctcttggagcccacggacgccggcggtcccccgcgaagcgcgcccgaggctgcctccatgttggccgctgagctgcgcggcaagactcgcagcctgggccgcgccgaggtggccctgggcgcgcagggccccagggaaaagccggcgcccccacagaaagccaagcgctccgtgccgccagcctcgcccgcccgcgcgcccccagcgaccgaaaccccggggcctgagaaggcggcgaccgacttgcccgcgcctgagaccccccggaagaagacccccatccagaagccgccgcgcaagaagagccgggaggcggcgggcgagctgggcagggcgggcgcacccaccctgtag

SEQ ID NO: 63 - protein, 822 aa, Homo sapiens
MKPALLPWALLLLATALGPGPGPTADAQESCSMRCGALDGPCSCHPTCSGLGTCCLDFRDFCLEILPYSGSMMGGKDFVVRHFKMSSPTDASVICRFKDSIQTLGHVDSSGQVHCVSPLLYESGRIPFTVSLDNGHSFPRAGTWLAVHPNKVSMMEKSELVNETRWQYYGTANTSGNLSLTWHVKSLPTQTITIELWGYEETGMPYSQEWTAKWSYLYPLATHIPNSGSFTFTPKPAPPSYQRWRVGALRIIDSKNYAGQKDVQALWTNDHALAWHLSDDFREDPVAWARTQCQAWEELEDQLPNFLEELPDCPCTLTQARADSGRFFTDYGCDMEQGSVCTYHPGAVHCVRSVQASLRYGSGQQCCYTADGTQLLTADSSGGSTPDRGHDWGAPPFRTPPRVPSMSHWLYDVLSFYYCCLWAPDCPRYMQRRPSNDCRNYRPPRLASAFGDPHFVTFDGTNFTFNGRGEYVLLEAALTDLRVQARAQPGTMSNGTETRGTGLTAVAVQEGNSDVVEVRLANRTGGLEVLLNQEVLSFTEQSWMDLKGMFLSVAAGDRVSIMLASGAGLEVSVQGPFLSVSVLLPEKFLTHTHGLLGTLNNDPTDDFTLHSGRVLPPGTSPQELFLFGANWTVHNASSLLTYDSWFLVHNFLYQPKHDPTFEPLFPSETTLNPSLAQEAAKLCGDDHFCNFDVAATGSLSTGTATRVAHQLHQRRMQSLQPVVSCGWLAPPPNGQKEGNRYLAGSTIYFHCDNGYSLAGAETSTCQADGTWSSPTPKCQPGRSYAVLLGIIFGGLAVVAAVALVYVLLRRRKGNTHVWGAQP

SEQ ID NO: 64 - DNA, 2469 bp, Homo sapiens
atgaagccagccctcctgccctgggccctgctgctgctggcgacagccctcggcccgggccccggacccacagcagatgcccaagagagctgctccatgcgctgtggcgccctggacgggccatgttcctgccacccgacgtgctctggccttggcacctgctgcttggatttccgggacttctgcctggagatattgccctactcaggatccatgatgggcggcaaggactttgtggtgcggcacttcaagatgtccagccccacagacgccagtgtgatctgcaggtttaaggacagcatccagaccctcggccatgtggactcctccgggcaagtgcactgtgtgtcacctctgctctatgagagcggccgcatccccttcactgtgtcactggacaacggccactccttccctcgtgcgggcacctggctggctgtgcaccccaacaaagtgtcaatgatggagaagagcgagttggtgaacgagacgcgttggcaatactacggcaccgccaacacctcaggcaacctcagcctgacctggcatgtcaagtcgctgcccacgcagaccatcaccatcgaactgtggggctacgaggagacaggaatgccctactcacaggagtggactgcaaagtggtcgtacctgtaccccctggccacacacatccccaactccggctctttcactttcaccccaaaacctgctcctcccagctaccagagatggcgagtgggtgcacttcggatcatcgacagcaaaaattacgcagggcagaaggacgtgcaggcgctctggaccaacgaccacgcactggcctggcacctgagcgatgacttccgagaggaccctgtggcctgggcacgaactcagtgccaggcctgggaggagctggaggatcagctgcccaacttcctggaggagctgccggactgcccctgcaccctgacccaggcccgggctgactccggccgcttcttcacggactacggctgtgacatggagcagggcagcgtgtgcacctaccaccccggggccgtgcactgtgtgcgttctgtgcaggccagcctccggtacggctcaggtcagcagtgctgctacacagcggacgggacgcagctcctgacagctgactccagcggcggcagcactcccgaccgcggccatgactggggcgcacccccgttccgcacgccaccccgagtgcccagcatgtcccactggctctacgatgtcctcagcttctattactgctgcctctgggcacccgactgcccccgctacatgcaacggcggccctccaatgactgccgcaactaccggcccccaagactggcctccgccttcggagacccacactttgtgaccttcgacggcaccaacttcacattcaatgggcgcggagagtacgtgctgctggaggcagcgctgaccgacctgagggtgcaggcgcgggcccagcccgggacgatgtccaacggcacggagacccgtggcactgggctgaccgcagtggccgtccaggagggcaactcagatgtggtggaagtcaggctggccaacaggaccggaggtctggaggtgctgctgaaccaggaggtgctgagcttcaccgagcagagctggatggacctgaaaggaatgttcctgtcggtggctgccggggacagggtctccatcatgctggcatcaggggccggcctggaggtcagcgtgcagggcccgttcctgagtgtgtccgtcctgctgcctgagaagttcctcacccacacccacggcctcctcgggacactcaacaacgaccccaccgacgacttcaccctgcacagcgggcgcgtcctgcccccaggcaccagtccccaggagctgttcctgtttggggccaactggaccgtgcacaatgcgtcctccctgctcacctacgattcctggttcctggtccacaacttcctgtaccaacccaagcacgaccccaccttcgagcccctcttccccagtgagaccaccctcaaccccagcctggcacaagaggcagccaaactatgtggggacgatcatttctgcaactttgatgtggcagccactgggagcctgagcacgggcactgccactcgggtggcccaccagctgcaccagcgtcgcatgcagagcctgcagccagtggtgtcctgtggctggctggccccacctcccaacggacaaaaggagggcaacaggtacctggcgggttccaccatctacttccactgtgacaacggctacagcctggccggggcagagaccagcacctgccaggctgacggcacctggtcctcacccaccccgaagtgccagccaggacgcagctacgcggtgctgttgggcatcatctttgggggcctcgcggtggtggcggcggttgcgctcgtctatgtgctgctgcgccgcaggaagggcaacacgcacgtctggggtgcacagccctga

SEQ ID NO: 65 - protein, 188 aa, Homo sapiens
MIIYRDLISHDEMFSDIYKIREIADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKCVSTRKWVKINNVKKTF

SEQ ID NO: 66 - DNA, 567 bp, Homo sapiens
atgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgtgtaagtacaaggaagtgggttaaaataaataatgtaaaaaagacattttag

SEQ ID NO: 67 - protein, 401 aa, Homo sapiens [sequence omitted]

SEQ ID NO: 68 - DNA, 1206 bp, Homo sapiens
atggttctctgttgtagaggttctctgttgtggatggttctctgttgtggatggttctctgttatagagggtctctgttgtggagggtctctgttgtggatggttctctgttgtgggtgggtctctgttgtgggtgggtctctgttgtggagggttctctgttgtggagggttctctgttgtggagggttctctgttgtggagggtctctgttggggatggttctctgttgtggaggctctctgttgtggaggttctctgttgtggaggatctctgttgtggatggttctgtgttgtggagggttctgtgttgtggagggttctctgttgtggagggtctctgttgtggatggttctctgtcgtggaggctctctgtcgtggaggctccctgtcgtggaggctctctgtcgtggagggtctctgtcgtgggtggttctttgttgtggagggtctctgttgtggatggttctctgttgtggagggtctctgttgtggagggtctctgttgtggagggttctctgttgtggagggtctctgttgtggagggttctctgttgtggagggttgtctgttgtggagggttctctgttgtggagcgtctctgttgtggagagtctctgttgtggagggttctctgttgtggagagtctgttgtggagggttctctgttgtggagagtctctgttgtggatggttctctgttgtggagggttctctgttgtggagggttctctgttgtggatggttctctgttatggagggttctctgttgtggagggttctctcttgtggatggttctctgttgtggatggttctctgttatggagggtctctgttgtggagggtctctgttgtggagagtctgttgtggatggttctctgttgtggagagtctctgttgtggagggtctctgttgtgggtggttctttgttgtgggtggttctctgttgtggatggttctttgttgtgggtggttctctgttgtgaatggttctttgttgtggatgggtctctgttgtggagggttctctgttgtggatgggtctctgttgtggatgggtctctgttgtggagggtctctgttgtggatggttctctgttttggatgggtctctgttgtggaggttctctgttgtggaagttctttgttgtatttgctgcaaatatgcatgataagatgcttaggagcatctacccactag

SEQ ID NO: 69 - protein, 290 aa, Homo sapiens
DRSRWRGRAGQGFGLRRREMAAGGRMEDGSLDITQSIEDDPLLDAQLLPHHSLQAHFRPRFHPLPTVIIVNLLWFIHLVFVVLAFLTGVLCSYPNPNEDKCPGNYTNPLKVQAVIILGKVILWILHLLLECYIQYHHSKIRNRGYNLIYRSTRHLKRLALMIQSSGNTVLLLILCMQHSFPKPGRLYLDLILAILALELICSLICLLIYTVKIRRFNKAKPEPDILEEEKIYAYPSNITSETGFRTISSLEEIVEKQGDTIEYLKRHNALLSKRLLALTSSDLGCQPSRT

SEQ ID NO: 70 - DNA, 873 bp, Homo sapiens
gacaggtccaggtggagaggccgggctggccagggcttcggcctccggcgtcgggaaatggcggcggggggcaggatggaggacggttccttggatatcacccagagtattgaagacgacccacttctggatgcccagcttctcccacaccactcattacaagctcactttagaccccgattccatcctcttcctacagtcatcatagtgaatcttctgtggtttattcatctcgtgtttgttgttttagcatttttaacaggtgtgctttgttcttatcctaatccaaatgaggacaagtgcccaggaaattacacaaacccattgaaagttcaggcggttataatccttgggaaagttattttgtggattctccatttactccttgaatgctacatccagtatcaccacagcaaaatcagaaaccgaggctataacttgatctaccgatcaacaaggcatctcaagagacttgcgttgatgatacagtcctctggcaacacagtgcttctcctcatactgtgcatgcagcactccttcccaaagcctggcagattgtatcttgacctcattctggccatcttggcactggaactcatctgttccctgatatgtctcctcatttacacagtgaaaatccggagatttaataaagctaaaccagagcctgatatacttgaagaagaaaaaatctatgcttaccccagcaatattacctcggagactggattcagaactatttcaagcctagaagaaattgttgaaaagcaaggagacaccattgaatacctgaagcgacacaatgcgctgctgagtaagcgattgttggctctcacttcctcagacctgggctgtcagccaagtagaacgtga

SEQ ID NO: 71 - protein, 257 aa, Homo sapiens
MAADGVDERSPLLSASHSGNVTPTAPPYLQESSPRAELPPPYTAIASPDASGIPVINCRVCQSLINLDGKLHQHVVKCTVCNEATPIKNPPTGKKYVRCPCNCLLICKDTSRRIGCPRPNCRRIINLGPVMLISEEQPAQPALPIQPEGTRVVCGHCGNTFLWMELRFNTLAKCPHCKKISSVGSALPRRRCCAYITIGMICIFIGVGLTVGTPDFARRFRATYVSWAIAYLLGLICLIRACYWGAIRVSYPEHSFA

SEQ ID NO: 72 - DNA, 774 bp, Homo sapiens
atggctgctgatggggtggacgaacgctcgcctctgctgtcagcatcccactccggaaatgtcactcccaccgccccaccgtacttgcaagaaagcagccccagagcggagctcccacctccatatacagccattgccagtccagacgccagtggtattccagtaataaactgccgtgtgtgccaatcactaatcaatttggatggcaagcttcaccagcatgtggttaagtgcacagtttgcaatgaagctacgccaatcaaaaaccccccaacaggcaagaaatatgttagatgcccttgtaattgtcttctcatttgtaaggacacatctcggcgaataggatgcccaagacccaactgtagacggataattaaccttggcccagtaatgcttatttctgaagaacaaccagctcagcctgcattgccaatccaaccagaaggtacaagggtcgtgtgtgggcactgtggaaacacattcctgtggatggaactgaggttcaacactctggcaaaatgcccacactgcaaaaaaatctcctcagtgggtagtgcacttccacgaagacgctgctgtgcatatattaccattggaatgatatgtattttcattggagttgggttaactgttggcaccccagattttgcaaggcgatttcgagcaacctatgtttcttgggcaattgcttatctcctaggattgatctgccttatccgagcttgttattggggagccataagagtcagttatccagaacacagttttgcataa

SEQ ID NO: 73 - protein, 118 aa, Homo sapiens [sequence omitted]

SEQ ID NO: 74 - DNA, 357 bp, Homo sapiens
atggtgcttacagccatggtgggaaaaatccataggaaaagactcacatacacaaatgcaggcagaataaaaaaactcacacaaacaaacgttgcagatgttgttaagttacataaaggagatatgcatggctgtgcatggtggctcgtgcctgtaatcttagcacttgggggggccggggcgggtggatcacttgaggccaggagctcaagaccagcctggccaacatggcgaagccctgtctctactaaaaatacaagagttggccaggcatggtggtccatgcctgtgatctcggctacttgggagactgaggtgggaggatcgcttgggcccaggaggcagagggtacagtga

SEQ ID NO: 75 - protein, 608 aa, Homo sapiens
MALRGPAGLGPGSRRPLDEAVAGAEGREAPALVAAGGAPEDDEEDDGRGRGLLRWDSFSAWLHCVCVVGFDLELGQAVEVIYPQHSKLTDREKTNICYLSFPDSNSGCLGDTQFCFRFRQSSGRRVSLHCLLDQFDKDLPVYLKKDPAYFYGYVYFRQVRDKTLKRGYFQKSLVLISKLPYIHFFHTVLKQIAPEYFEKNEPYLEAACNDVDRWPAPVPGKTLHLPIMGVVMKVRIPTCHDKPGTTQIVQLTQQVDTNISVILPTVHEVDIFRCFCPVFLHSQMLWELVLLGEPLVVMAPSPSESSETVLALVNCISPLKYFSDFRPYFTIHDSEFKEYTTRTQAPPSVILGVTNPFFAKTLQHWPHIIRIGDLKPTGEIPKQVKVKKLKNLKTLDSKPGVYTSYKPYLNRDEEIIKQLQKGVQQKRPSEAQSVILRRYFLELTQSFIIPLERYVASLMPLQKSISPWKSPPQLRQFLPEEFMKTLEKTGPQLTSRIKGDWIGLYRHFLKSPNFDGWFKTRRKEMTQKLEALHLEALCEEDLLLWIQKHTEVETVDLVLKLKNKLLQADREHLPVKPDTMEKLRTHIDAIILALPEDLQGILLKTGMT

SEQ ID NO: 76 - DNA, 1827 bp, Homo sapiens
atggctttgaggggccctgcgggcttggggcccggctctcgaaggccgttggacgaagcggtggcaggggccgagggccgcgaggcgccggcccttgtggcggcgggaggcgcgccagaggacgatgaagaggacgatggccgtggccggggcctgctgcgctgggacagcttctccgcctggctgcactgcgtgtgtgtggtgggcttcgacctggagctgggccaggccgtggaggtaatttatcctcagcattccaaacttactgacagagaaaaaaccaatatttgctatttgtcttttccagattcaaattcaggttgtcttggagatacccagttttgttttagatttcgacagtcttctgggaggagggtgtcgctgcattgtctcctggatcaatttgacaaagatttaccagtttacttaaagaaggatcctgcttatttttatggatatgtgtatttccgacaagttcgagataaaactctaaaaagaggctactttcagaagtccttggttttgatcagcaaactaccttatattcatttttttcacactgtgctcaaacagatagcaccagagtattttgaaaagaatgaaccttatttggaagcagcttgtaatgatgttgatcgatggcctgccccagtgccagggaaaacattacacctgccaatcatgggggtggtaatgaaggtacggattcccacatgtcatgacaagcctgggacaactcaaatagtgcagttaactcagcaggtggacacaaatatatctgttattttacctactgttcatgaggtggatattttcaggtgtttctgcccagttttccttcatagtcagatgctctgggagctggtgctgttgggggagccccttgtggttatggcgccatcaccatcggaatcatcagagactgtattggcacttgttaactgtatttctccattaaagtacttcagtgatttccgaccttatttcactattcatgatagtgaattcaaagaatatactacccgtacgcaagctccgccctcagttatattaggagtaaccaaccctttttttgctaagacactccagcactggccacacattattcgaataggagaccttaaacctacaggtgaaattcctaagcaggttaaagtgaaaaaactgaagaatctaaagactctggattccaaacctggagtttatacttcatataagccatatttaaatagagatgaagagatcataaaacaattacagaagggtgtacaacagaaacgtccttctgaggctcaaagtgttattcttcgacgctattttttggaactgacacaaagtttcatcattccattagaaagatatgtggcaagcttgatgcctttgcagaaaagtatttccccatggaagagtccacctcaattaagacagtttcttccagaagaatttatgaaaacacttgagaaaacaggacctcagctaacctctagaataaaaggcgattggattggactttaccggcatttcctaaagtctccaaattttgatggctggtttaagacccggaggaaggaaatgacccaaaaattggaggcactccatctagaagctctttgtgaagaggacttacttctctggatccagaaacacacagaagtagaaacagtagaccttgtcttgaagctgaaaaataagctgttgcaggctgatcgagagcacttacctgtgaaacctgacactatggaaaagttacggacacacatagatgccattatcttagcattgccagaggacttgcaaggcatactgctcaaaacgggcatgacatga

SEQ ID NO: 77 - protein, 175 aa, Homo sapiens
MGALVIRGIRNFNLENRAEREISKMKPSVAPRHPSTNSLLREQISLYPEVKGEIARKDEKLLSFLKDVYVDSKDPVSSLQVKAAETCQEPKEFRLPKDHHFDMINIKSIPKGKISIVEALTLLNNHKLFPETWTAEKIMQEYQLEQKDVNSLLKYFVTFEVEIFPPEDKKAIRSK

SEQ ID NO: 78 - DNA, 528 bp, Homo sapiens
atgggagcactagtgattcgcggtatcaggaatttcaacctagagaaccgagcggaacgggaaatcagcaagatgaagccctctgtcgctcccagacacccctctaccaacagcctcctgcgagagcagattagtctctatccagaagttaaaggagagattgctcgtaaagatgaaaagctgctgtcgtttctaaaagatgtgtatgttgattccaaagatcctgtgtcttccttgcaggtaaaagctgctgaaacatgtcaagagccgaaggaattcagattgccgaaagaccatcattttgatatgataaatattaagagcattcccaaaggcaaaatttccattgtagaagcattgacacttctcaataatcataagcttttcccagaaacctggactgctgagaaaataatgcaagaataccagttagaacagaaagatgtgaattctcttcttaaatattttgttacttttgaagtcgaaatcttccctcctgaagacaagaaagcaatacgatcaaaatga

SEQ ID NO: 79 - protein, 132 aa, Homo sapiens [sequence omitted]

SEQ ID NO: 80 - DNA, 399 bp, Homo sapiens
atggtattttattgtatgcacttaaagtattattctgagaaggctccaaaggggccacagggcaaaaacaattacgaccctattgggctcggaacacaataccccaaagtatggcactttggcatgctgagtgctttaaaccaaaggagattggagggcctcagacgcaaagtctctctgaacctctcctactctcctgtctctgacttctttttcccctacaaaggaagtcatagaaaccaaaattcctcctcagtctattaccatgagatcatacccttttgtccagtcacatttctacaaggctgcccattcttcatggaatgtaagcataaaaatagacagttttggctgggcggggtggctcgggcctgtaatcccagcactttgggtggctga

SEQ ID NO: 81 - protein, 186 aa, Homo sapiens
MAGAEWKSLEECLEKHLPLPDLQEVKRVLYGKELRKLDLPREAFEAASREDFELQGYAFEAAEEQLRRPRIVHVGLVQNRIPLPANAPVAEQVSALHRRIKAIVEVAAMCGVNIICFQEAWILRPHHQEPRPPCCYAPSCCLIPSVFPHRSESIRSSPSLYHHLVQAPGSSPLAFVIPSSWSPVPS

SEQ ID NO: 82 - DNA, 561 bp, Homo sapiens
atggcgggcgctgagtggaagtcgctggaggaatgcttggagaagcacctgccgctccccgacttgcaggaagtgaagcgcgttctctatggcaaggaactcaggaagcttgatctgcccagggaagctttcgaagctgcctccagagaagactttgaactgcagggatatgcctttgaagcagcggaggagcagctgagacgaccccgcattgtgcacgtggggctggttcagaacagaatccccctccccgcaaatgcccctgtggcagaacaggtctctgcccttcatagacgcataaaggctatcgtagaggtggctgcaatgtgtggagtcaacatcatctgtttccaggaagcatggattctccgtccacatcaccaggaaccccggcctccctgctgttacgcaccgagctgttgtctgattccttcagttttcccacatcgttctgaatccattcgctcctctccatctctctaccaccacctggtccaagcccctggcagctcacctctggcttttgtaattccctccagctggtctcctgtgccctcctga

SEQ ID NO: 83 - protein, 384 aa, Homo sapiens
MAGAEWKSLEECLEKHLPLPDLQEVKRVLYGKELRKLDLPREAFEAASREDFELQGYAFEAAEEQLRRPRIVHVGLVQNRIPLPANAPVAEQVSALHRRIKAIVEVAAMCGVNIICFQEAWTMPFAFCTREKLPWTEFAESAEDGPTTRFCQKLAKNHDMVVVSPILERDSEHGDVLWNTAVVISNSGAVLGKTRKNHIPRVGDFNESTYYMEGNLGHPVFQTQFGRIAVNICYGRHHPLNWLMYSINGAEIIFNPSATIGALSESLWPIEARNAAIANHCFTCAINRVGTEHFPNEFTSGDGKKAHQDFGYFYGSSYVAAPDSSRTPGLSRSRDGLLVAKLDLNLCQQVNDVWNFKMTGRYEMYARELAEAVKSNYSPTIVKE

SEQ ID NO: 84 - DNA, 1155 bp, Homo sapiens
atggcgggcgctgagtggaagtcgctggaggaatgcttggagaagcacctgccgctccccgacttgcaggaagtgaagcgcgttctctatggcaaggaactcaggaagcttgatctgcccagggaagctttcgaagctgcctccagagaagactttgaactgcagggatatgcctttgaagcagcggaggagcagctgagacgaccccgcattgtgcacgtggggctggttcagaacagaatccccctccccgcaaatgcccctgtggcagaacaggtctctgcccttcatagacgcataaaggctatcgtagaggtggctgcaatgtgtggagtcaacatcatctgtttccaggaagcatggactatgccctttgccttctgtacgagagagaagcttccttggacagaatttgctgagtcagcagaggatgggcccaccaccagattctgtcagaagctggcgaagaaccatgacatggtggtggtgtctcccatcctggaacgagacagcgagcatggggatgttttgtggaatacagccgtggtgatctccaattccggagcagtcctgggaaagaccaggaaaaaccacatccccagagtgggtgatttcaacgagtcaacttactacatggagggaaacctgggccaccccgtgttccagacgcagttcggaaggatcgcggtgaacatttgctacgggcggcaccaccccctcaactggcttatgtacagcatcaacggggctgagatcatcttcaacccctcggccacgataggagcactcagcgagtccctgtggcccatcgaggccagaaacgcagccattgccaatcactgcttcacctgcgccatcaatcgagtgggcaccgagcacttcccgaacgagtttacctcgggagatggaaagaaagctcaccaggactttggctacttttatggctcgagctatgtggcagcccctgacagcagccggactcctgggctgtcccgtagccgggatggactgctagttgctaagctcgacctaaacctctgccagcaggtgaatgatgtctggaacttcaagatgacgggcaggtatgagatgtacgcacgggagctcgccgaagctgtcaagtccaactacagccccaccatcgtgaaagagtag

SEQ ID NO: 85 - protein, 254 aa, Homo sapiens
MNSRQAWRLFLSQGRGDRWVSRPRGHFSPALRREFFTTTTKEGYDRRPVDITPLEQRKLTFDTHALVQDLETHGFDKTQAETIVSALTALSNVSLDTIYKEMVTQAQQEITVQQLMAHLDAIRKDMVILEKSEFANLRAENEKMKIELDQVKQQLMHETSRIRADNKLDINLERSRVTDMFTDQEKQLMETTTEFTKKDTQTKSIISETSNKIDAEIASLKTLMESNKLETIRYLAASVFTCLAIALGFYRFWK

SEQ ID NO: 86 - DNA, 765 bp, Homo sapiens
atgaatagtcgccaggcttggcggctctttctctcccaaggcagaggagatcgttgggtttcaaggccccgcgggcatttctcgccggccctgcggagagagttcttcactaccacaaccaaggagggatatgataggcggccagtggatataactcctttagaacaaaggaaattaacttttgatacccatgcattggttcaggacttggaaactcatggatttgacaaaacacaagcagaaacaattgtatcagcgttaactgctttatcaaatgtcagcctggatactatctataaagagatggtcactcaagctcaacaggaaataacagtacaacagctaatggctcatttggatgctatcaggaaagacatggtcatcctagagaaaagtgaatttgcaaatctgagagcagagaatgagaaaatgaaaattgaattagaccaagttaagcaacaactaatgcatgaaaccagtcgaatcagagcagataataaactggatatcaacttagaaaggagcagagtaacagatatgtttacagatcaagaaaagcaacttatggaaacaactacagaatttacaaaaaaggatactcaaaccaaaagtattatttcagagaccagtaataaaattgacgctgaaattgcttccttaaaaacactgatggaatctaacaaacttgagacaattcgttatcttgcagcttcggtgtttacttgcctggcaatagcattgggattttatagattctggaagtag

SEQ ID NO: 87 - protein, 254 aa, Homo sapiens
MNSRQAWRLLLSQGRGDRWVSRPRGHFSPALRREFFTTTTKEGYDRRPVDITPLEQRKLTFDTHALVQDLETHGFDKTQAETIVSALTALSNVSLDTIYKEMVTQAQQEITVQQLMAHLDAIRKDMVILEKSEFANLRAENEKMKIELDQVKQQLMHETSRIRADNKLDINLERSRVTDMFTDQEKQLMETTTEFTKKDTQTKSIISETSNKIDAEIASLKTLMESNKLETIRYLAASVFTCLAIALGFYRFWK

SEQ ID NO: 88 - DNA, 765 bp, Homo sapiens
atgaatagtcgccaggcttggcggctcttgctctcccaaggcagaggagatcgttgggtttcaaggccccgcgggcatttctcgccggccctgcggagagagttcttcactaccacaaccaaggagggatatgataggcggccagtggatataactcctttagaacaaaggaaattaacttttgatacccatgcattggttcaggacttggaaactcatggatttgacaaaacacaagcagaaacaattgtatcagcgttaactgctttatcaaatgtcagcctggatactatctataaagagatggtcactcaagctcaacaggaaataacagtacaacagctaatggctcatttggatgctatcaggaaagacatggtcatcctagagaaaagtgaatttgcaaatctgagagcagagaatgagaaaatgaaaattgaattagaccaagttaagcaacaactaatgcatgaaaccagtcgaatcagagcagataataaactggatatcaacttagaaaggagcagagtaacagatatgtttacagatcaagaaaagcaacttatggaaacaactacagaatttacaaaaaaggatactcaaaccaaaagtattatttcagagaccagtaataaaattgacgctgaaattgcttccttaaaaacactgatggaatctaacaaacttgagacaattcgttatcttgcagcttcggtgtttacttgcctggcaatagcattgggattttatagattctggaagtag

SEQ ID NO: 89 - protein, 353 aa, Homo sapiens
MRLPRRAALGLLPLLLLLPPAPEAAKKPTPCHRCRGLVDKFNQGMVDTAKKNFGGGNTAWEEKTLSKYESSEIRLLEILEGLCESSDFECNQMLEAQEEHLEAWWLQLKSEYPDLFEWFCVKTLKVCCSPGTYGPDCLACQGGSQRPCSGNGHCSGDGSRQGDGSCRCHMGYQGPLCTDCMDGYFSSLRNETHSICTACDESCKTCSGLTNRDCGECEVGWVLDEGACVDVDECAAEPPPCSAAQFCKNANGSYTCEECDSSCVGCTGEGPGNCKECISGYAREHGQCADVDECSLAEKTCVRKNENCYNTPGSYVCVCPDGFEETEDACVPPAEAEATEGESPTQLPSREDL

SEQ ID NO: 90 - DNA, 1062 bp, Homo sapiens
atgcgcctgccgcgccgggccgcgctggggctcctgccgcttctgctgctgctgccgcccgcgccggaggccgccaagaagccgacgccctgccaccggtgccgggggctggtggacaagtttaaccaggggatggtggacaccgcaaagaagaactttggcggcgggaacacggcttgggaggaaaagacgctgtccaagtacgagtccagcgagattcgcctgctggagatcctggaggggctgtgcgagagcagcgacttcgaatgcaatcagatgctagaggcgcaggaggagcacctggaggcctggtggctgcagctgaagagcgaatatcctgacttattcgagtggttttgtgtgaagacactgaaagtgtgctgctctccaggaacctacggtcccgactgtctcgcatgccagggcggatcccagaggccctgcagcgggaatggccactgcagcggagatgggagcagacagggcgacgggtcctgccggtgccacatggggtaccagggcccgctgtgcactgactgcatggacggctacttcagctcgctccggaacgagacccacagcatctgcacagcctgtgacgagtcctgcaagacgtgctcgggcctgaccaacagagactgcggcgagtgtgaagtgggctgggtgctggacgagggcgcctgtgtggatgtggacgagtgtgcggccgagccgcctccctgcagcgctgcgcagttctgtaagaacgccaacggctcctacacgtgcgaagagtgtgactccagctgtgtgggctgcacaggggaaggcccaggaaactgtaaagagtgtatctctggctacgcgagggagcacggacagtgtgcagatgtggacgagtgctcactagcagaaaaaacctgtgtgaggaaaaacgaaaactgctacaatactccagggagctacgtctgtgtgtgtcctgacggcttcgaagaaacggaagatgcctgtgtgccgccggcagaggctgaagccacagaaggagaaagcccgacacagctgccctcccgcgaagacctgtaa

SEQ ID NO: 91 - protein, 321 aa, Homo sapiens
MRLPRRAALGLLPLLLLLPPAPEAAKKPTPCHRCRGLVDKFNQGMVDTAKKNFGGGNTAWEEKTLSKYESSEIRLLEILEGLCESSDFECNQMLEAQEEHLEAWWLQLKSEYPDLFEWFCVKTLKVCCSPGTYGPDCLACQGGSQRPCSGNGHCSGDGSRQGDGSCRCHMGYQGPLCTDCMDGYFSSLRNETHSICTACDESCKTCSGLTNRDCGECEVGWVLDEGACVDVDECAAEPPPCSAAQFCKNANGSYTCEDVDECSLAEKTCVRKNENCYNTPGSYVCVCPDGFEETEDACVPPAEAEATEGESPTQLPSREDL

SEQ ID NO: 92 - protein, 284 aa, Homo sapiens
MRLPRRAALGLLPLLLLLPPAPEAAKKPTPCHRCRGLVDKFNQGMVDTAKKNFGGGNTAWEEKTLSKYESSEIRLLEILEGLCESSDFECNQMLEAQEEHLEAWWLQLKSEYPDLFEWFCVKTLKVCCSPGTYGPDCLACQGGSQRPCSGNGHCSGDGSRQGDGSCRCHMGYQGPLCTDCMDGYFSSLRNETHSICTACDESCKTCSGLTNRDCGECEVGWVLDEGACVDVDECAAEPPPCSAAQFCKNANGSYTCEGGPGGRVCTPGPAGFRCCLCQHSFMAS

SEQ ID NO: 93 - protein, 325 aa, Homo sapiens
MRLPRRAALGLLPLLLLLPPAPEAAKKPTPCHRCRGLVDKFNQGMVDTAKKNFGGGNTAWEEKTLSKYESSEIRLLEILEGLCESSDFECNQMLEAQEEHLEAWWLQLKSEYPDLFEWFCVKTLKVCCSPGTYGPDCLACQGGSQRPCSGNGHCSGDGSRQGDGSCRCHMGYQGPLCTDCMDGYFSSLRNETHSICTACDESCKTCSGLTNRDCGECEVGWVLDEGACVECDSSCVGCTGEGPGNCKECISGYAREHGQCADVDECSLAEKTCVRKNENCYNTPGSYVCVCPDGFEETEDACVPPAEAEATEGESPTQLPSREDL

SEQ ID NO: 94 - protein, 402 aa, Homo sapiens
MRLPRRAALGLLPLLLLLPPAPEAAKKPTPCHRCRGLVDKFNQGMVDTAKKNFGGGNTAWEEKTLSKYESSEIRLLEILEGLCESSDFECNQMLEAQEEHLEAWWLQLKSEYPDLFEWFCVKTLKVCCSPGTYGPDCLACQGGSQRPCSGNGHCSGDGSRQGDGSCRCHMGYQGPLCTDCMDGYFSSLRNETHSICTVRTGLSDSYPPCCLSLGCWRGVGHAWIRGRNTHTQPGYSSRVWIAAFSPACDESCKTCSGLTNRDCGECEVGWVLDEGACVDVDECAAEPPPCSAAQFCKNANGSYTCEECDSSCVGCTGEGPGNCKECISGYAREHGQCADVDECSLAEKTCVRKNENCYNTPGSYVCVCPDGFEETEDACVPPAEAEATEGESPTQLPSREDL

SEQ ID NO: 95 - protein, 373 aa, Homo sapiens
MRLPRRAALGLLPLLLLLPPAPEAAKKPTPCHRCRGLVDKFNQGMVDTAKKNFGGGNTAWEEKTLSKYESSEIRLLEILEGLCESSDFECNQMLEAQEEHLEAWWLQLKSEYPDLFEWFCVKTLKVCCSPGTYGPDCLACQGGSQRPCSGNGHCSGDGSRQGDGSCRCHMGYQGPLCTDCMDGYFSSLRNETHSICTACDESCKTCSGLTNRDCGECEVGWVLDEGACVDVDECAAEPPPCSAAQFCKNANGSYTCEECDSSCVGCTGEGPGNCKECISGYAREHGQCADVDECSLAEKTCVRKNENCYNTPGSYVCVCPDGFEETEDACVPPAEAGEWHGCPPHRLPSPGPQGLHVDWLLGLKSTQMVALRW

SEQ ID NO: 96 - protein, 2204 aa, Homo sapiens [sequence omitted]

SEQ ID NO: 97 - DNA, 6615 bp, Homo sapiens
atgcctctgttcttctccgcgctgttggtcttgctgctagttgcgcttagcgccctctttctaggccggtggcttgtggtccggttggccaccaagtggtgtcagcggaagctgcaggcggagctaaagattggctccttccgctttttttggatccagaatgtcagtcttaagtttcagcaacaccagcaaacagtggaaattgataacctgtggatttccagcaaaccccttagccatgatcttccacactatgtggcattgtgctttggagaagtgcgtatcagaacggacctacagaaagtttctgacctgtctgccccattctcccagagcgctggggtggatcaaaaggaactgtccttcagcccatccttattgaagatcttctgccaactattctccattcatgtagatgctataaacatcatggttctcaaggtggatacctctgagtccttatggcatattcagatcagtagaagcagatttcttttggatagtgatgggaaaaggctaatctgtgaggtgagcttatgtaagatcaacagcaaagttctaaagagtggtcagctggaggacacctgcctagtggagctttcactggccctggacctgtgtctaaaggtgggcattagcagtcggcatctcactgctatcactgtggatgtgtggacactccatgctgaactgcatgagggcctcttccagagccaactgctgtgccagggcccaagcctagcatctaagcctgttccctgttcagaggtgacagaaaacttagttgagccaactctgcctggcctattccttctccagcagctgccagaccaggtcaaggttaagatggagaacacaagcgtggtattgtccatgaatagtcaaaagaggcacctgacttggactctgaagctgctgcagttcctgtaccaccgtgatgaggatcagctgccccttcgaagcttcacagcaaactctgatatggcacagatgagcactgaactgctgctggaagatgggttgttgttgtcccagagtcgccaacgcattgtctgcctcaactccctcaaggctagtgtgcaggtgaccaccattgacctctcagcctccctagttctgaacacttgcatcattcactaccggcaccaggaattctctcactggctgcacctgctagcactggaaacccaagggtctagttcacctgttctaaagcaaaggaaaaaaagaaccttcccccaaatcctggctcccatcatctttagcacctccatctccaatgtcaacatttccattcaacttggagatacaccaccttttgccttgggattcaattctatctctctggattaccagcacctcaggccacaaagcatccatcagcggggcgtcctaactgtggaccacctctgctggcgtgtgggcagtgactcccacattcagcgggcgccacacccacccaatatgcatgtttggggtgaggcacttgttctggactccttcacactacagggtagctataaccagcctctggtcctgtccagcacccagtcagataccctttttcttgattgtaccattcgaggacttcaggtggaagcatcagatacctgtgcccaatgtctgtctcgtatcttatccctgatgggtccacaatctgggaagtcagctgtctctaggcactcttcatttggggaatctgtgtcattactgtggaaggtggacttgaaggtcgaagacatgaacttgtttaccctttctgccttggttggtgcttcagaggtacgactggacaccctagctatcctgggcagtgcagagacgtccactgtggggattcaaggacttgtgttagcgctggtgaaatcagtcacggagaagatgcaaccctgttgcaaggcccctgacatccctaccccagtgctcagcctttccatgctctccatcacctatcacagcagcatccgctctctggaggttcagtgtggtgcagggctgaccttactttggagccccccagatcacatgtacctgtaccagcatgtcctggccactctacagtgccgagacctactaagagccactgtgtttcctgagactgtaccatcccttgcactagagacttcaggaactacttctgagctagaaggccgtgcccctgagccattacccccaaagcggctgctaaacctaaccctggaggtgagcacagccaagctcacagcttttgtagctgaggacaagttcattaccctggctgcagagagtgtgtcactgagccggcatggaggttccctgcaggcatactgtccagagctggctgctggctttgatggcaatagtatcttcaacttcaaggaggtggaggtgcagctgctacctgagctggagtttccttatcagtatgacttttctcgaactctagatgaggctgtgggagttcagaagtggctgaagggactacatcaagggactcgtgcttgggcctctccaagccctgtcccactcccacctgatctactcttaaaggttgagcacttctcatgggttttcttggatgatgtttttgaggtgaaacttcatgataactacgagctgatgaaggatgaaagtaaggagagtgccaaaagactacagctactggatgctaaagtggccgcccttcggaagcagcatggggagttgttgcctgcccgcaaaattgaggagctctatgcctctttggaacgcaaaaacattgaaatctacatccagcgttcccgtcgtctctatggcaacacacccatgcgccgggcactgcttacttggagcttagcagggctagaactggtagctctggcagatgcctccttccatggtcctgagcatgtggtagaacaggttcaagagcttgatccaggcagcccttttccccctgagggattagatcttgtcattcagtggtgtcgaatgctcaagtgcaatgtcaagagctttctggttcggatcagggactatccacggtacctgtttgagatccgtgactggcggctaatgggtcgacttgtgggcaccgagcagagtggtcagccttgctcccgtcggcgtcagatcttgcacttggggcttccgtggggtaacgtggcagtggagaggaacatgcccccactcaaattctaccatgactttcactcggaaatattccagtacacagtggtgtggggcccatgctgggatccagcctggacactaattggccagtgtgtggacctcttgaccaagccctcagctgaccccagcccacctttgccctggtgggacaagagccgtcttctgttccatggagactggcacatggacattgaacaggcgaacctgcaccagctggccactgaggatccatacaacacaactgaaaatatgcactgggagtggagccacctgtcttttcattggaaacctggtcagtttgtgttcaagggtgacttggatatcaacgtgagaacagcctctaagtatgacgactgctgcttccttcacctgcctgacctctgcatgacactggacctgcagtggctgtgccatgggaacccccatgatcaccatagtgtcactctgcgggccccagagttcctgcctgaggtgcccttgggccagcttcatgactcctaccgggcctttcgctcggagaacctcaatctctccatcaagatggatctggctcggcacagtggaacaatatcccagccccgaattctgctatatagtagtaccctgcgctggatgcaaaacttctgggcaacttggacaagtgtcacaaggcctatctgcaggggaaagctcttcaataacctgaaacccagcaagaagaaacttggtcagcactacaagcaactttcctatacagccctctttccccagctgcaggtacattattgggcctcatttgcccagcaacggggcatccagattgagtgcagtcagggccatgtcttcactcgggggactcagcggcttatacctcaagcaggcacagtgatgcggcgccttatctctgattggagtgttacccagatggtgagtgacctaagtcaggtgaccgttcacctgatggcctcacccactgaagagaatgctgatcactgtcttgatcccttggtaacaaagacccacctgctgagcttgtcctccctcacctaccaacggcatagcaatcgcacagctgaggaggagctctctgctcgtgatggggatcctacctttcatacacatcagctgcacttagtagatttacggatttcctggacaactaccaatcgagacattgcctttgggttatatgatgggtacaaaaaggcagctgtactcaaacgtaatctttctactgaggccctgaaggggttaaagattgatccacagatgccagccaaaaagccaaagcggggtgtcccaactagtgcctcagccccacctcgtgttaacactcccagcttcagtggacaacctgataaggggtcatcaggaggtgcttacatgttgcagaagctaattgaagagacagataggcttgtagtgttcacagaagaggaatcaggcatgagtgaccagttgtgtggcattgctgcctgccagacggatgacatatacaaccgaaactgccttattgaattggtcaactgtcagatggttcttcgtggagcagagacagaaggctgtgtcattgtgtcagctgccaaagcccaactgctgcagtgccagcaccatccagcctggtatggtgatacattgaagcaaaagacatcctggacttgcctcttggatggcatgcagtactttgccaccactgaaagcagccccacagagcaggatggccgacagctctggttagaggtgaagaatatcgaggagcaccggcagcgtagtctggactctgtgcaggagctgatggagagtgggcaggcagtgggcggcatggttaccacaaccacagattggaaccagccagctgaggcacagcaagcccagcaagtccagcggatcatttcgcgttgcaactgccgaatgtactatattagttacagccatgacattgatcctgaactagcaactcagattaagccacctgaagttcttgagaaccaggaaaaggaagatctcctaaagaagcaggaaggggctgtggataccttcacacttatccaccatgagctggaaatttccaacccagctcagtatgccatgatcctggacattgtcaacaacctgctgctccatgtagaacctaagcggaaggaacatagtgagaagaagcaacgggtcaggttccagcttgagatctctagcaatccagaggagcaacgcagcagcatactgcatttgcaggaggctgtgcggcagcatgtggcccaaatacgacagctggagaagcagatgtattctaccatgaagtctttgcaggatgacagcaagaatgagaatctgcttgacctgaaccagaagcttcagttgcagctaaaccaggagaaggccaacctgcagctggaaagtgaagaactgaatatcctcatcaggtgttttaaggatttccaactgcagcgggctaacaagatggagctgcgaaagcagcaagaagatgtgagtgtggtccgtcgcactgagttttactttgctcaggcacggtggcgcctgacagaggaagatggacagctgggaattgctgaattagaactgcagaggttcctctacagcaaggtgaataagtctgatgacacagcagaacatcttctggagttgggctggtttaccatgaacaacctcctccccaatgctgtctataaggtagtactgcggccccagagctcctgccagtctgggcgacagctagctctccgcctcttcagcaaagttcggccccctgttgggggtatctctgttaaggagcattttgaggtaaatgtggtgcctctcaccatccagctgacacaccagttcttccacagaatgatgggctttttctttcctggccgaagtgtggaagatgatgaagttggtgatgaagaggataagtccaaactggtgactactggaataccagtggtgaagcctcggcagctgattgcaacagatgatgcagtaccactgggccctgggaagggtgtggcacagggtttgactcggagttctggggtcagaaggtcatttcgcaaatcgccagagcaccctgtggatgacattgacaagatgaaagagcgagctgccatgaacaactccttcatctacataaagattccacaggttccactgtgtgtcagctacaagggtgagaagaacagtgtggactggggcgaccttaacctggtgctgccctgtctggagtaccacaacaacacatggacatggctagactttgccatggctgtcaaaagggacagccgcaaagccctggttgcccaggtaatcaaagagaagctaaggctgaagtctgcaacgggctctgaggtccggggaaagctagaaactaaatcggacctgaacatgcaacagcaggaagaggaggagaaagcccggctcctcattggtttaagtgtgggcgacaagaaccctggcaagaagtccatctttggcaggcgcaaatga

SEQ ID NO: 98 - protein, 989 aa, Homo sapiens
MTMAGGRRGLVAPQNTFLENIVRRSNDTNFVLGNAQIVDWPIVYSNDGFCKLSGYHRAEVMQKSSTCSFMYGELTDKDTIEKVRQTFENYEMNSFEILMYKKNRTPVWFFVKIAPIRNEQDKVVLFLCTFSDITAFKQPIEDDSCKGWGKFARLTRALTSSRGVLQQLAPSVQKGENVHKHSRLAEVLQLGSDILPQYKQEAPKTPPHIILHYCVFKTTWDWIILILTFYTAILVPYNVSFKTRQNNVAWLVVDSIVDVIFLVDIVLNFHTTFVGPAGEVISDPKLIRMNYLKTWFVIDLLSCLPYDVINAFENVDEVSAFMGDPGKIGFADQIPPPLEGRESQGISSLFSSLKVVRLLRLGRVARKLDHYIEYGAAVLVLLVCVFGLAAHWMACIWYSIGDYEIFDEDTKTIRNNSWLYQLAMDIGTPYQFNGSGSGKWEGGPSKNSVYISSLYFTMTSLTSVGFGNIAPSTDIEKIFAVAIMMIGSLLYATIFGNVTTIFQQMYANTNRYHEMLNSVRDFLKLYQVPKGLSERVMDYIVSTWSMSRGIDTEKVLQICPKDMRADICVHLNRKVFKEHPAFRLASDGCLRALAMEFQTVHCAPGDLIYHAGESVDSLCFVVSGSLEVIQDDEVVAILGKGDVFGDVFWKEATLAQSCANVRALTYCDLHVIKRDALQKVLEFYTAFSHSFSRNLILTYNLRKRIVFRKISDVKREEEERMKRKNEAPLILPPDHPVRRLFQRFRQQKEARLAAERGGRDLDDLDVEKGNVLTEHASANHSLVKASVVTVRESPATPVSFQAASTSGVPDHAKLQAPGSECLGPKGGGGDCAKRKSWARFKDACGKSEDWNKVSKAESMETLPERTKASGEATLKKTDSCDSGITKSDLRLDNVGEARSPQDRSPILAEVKHSFYPIPEQTLQATVLEVRHELKEDIKALNAKMTNIEKQLSEILRILTSRRSSQSPQELFEISRPQSPESERDIFGAS

SEQ ID NO: 99 - DNA, 2970 bp, Homo sapiens
atgaccatggctgggggcaggaggggactagtggcccctcaaaacacgtttctggagaatattgttcggcggtccaatgatactaattttgtgttggggaatgctcagatagtggactggcctattgtgtacagcaatgatggattttgcaagctgtctggctatcacagggcagaagtgatgcaaaaaagcagcacctgcagttttatgtatggggagctgactgataaagacacgattgaaaaagtgcggcaaacatttgagaactatgagatgaattcctttgaaattctgatgtacaagaagaacaggacacctgtgtggttctttgtgaaaattgctccaattcgaaacgaacaggataaagtggttttatttctttgcactttcagtgacataacagctttcaaacagccaattgaggatgattcatgtaaaggctgggggaagtttgctcggctgacaagagcactgacaagcagcaggggtgtcctgcagcagctggctccaagcgtgcaaaaaggcgagaatgtccacaagcactcccgcctggcagaggtcctacagctgggctcagacatccttccccagtacaagcaagaggcaccaaagactccccctcacatcatcttacattattgtgtttttaagaccacgtgggattggatcatcttgatcttgaccttctatacagccatcttggtcccttataatgtctccttcaaaaccaggcagaataatgtggcctggctggttgttgatagcatcgtggatgttatctttttggtggacattgtgctcaattttcataccacctttgttggaccagcaggggaggtgatttctgaccccaaacttatccgcatgaactacctgaagacgtggtttgtgattgaccttctgtcctgtttgccatatgatgtcatcaacgcttttgagaacgtggatgaggttagtgcctttatgggtgatccagggaagattggttttgctgatcagattccaccaccactggaggggagagagagtcagggcatcagcagcctgttcagctctctaaaagttgtccggctgctccgtcttgggcgagtggcccgtaagctggaccactacattgaatatggagctgctgtgctggtcctgctggtgtgtgtgtttgggctggctgcacactggatggcctgcatctggtacagcattggggactatgagatctttgacgaggacaccaagacaatccgcaacaacagctggctgtaccaactagcgatggacattggcaccccttaccagtttaatgggtctggctcagggaagtgggaaggtggtcccagcaagaattctgtctacatctcctcgttgtatttcacaatgaccagcctcaccagtgtgggctttgggaacatcgccccatccacagacattgagaagatctttgcagtggccatcatgatgattggctcacttctctatgccaccatcttcgggaatgtgacgactattttccaacagatgtatgccaacaccaacagataccatgagatgctcaacagtgttcgggacttcctgaagctctaccaggtgccaaaaggattgagtgagcgagtaatggattatattgtgtccacttggtccatgtccagaggcattgacacagagaaggtcctgcagatctgccccaaggacatgagagccgacatctgcgtgcacctgaaccgcaaggtgttcaaggagcacccggccttccggctggccagtgatggctgcctccgggcactggccatggagttccagacggtgcactgtgccccaggggacctcatctaccatgcaggagagagcgttgacagcctctgctttgtggtttctggctccctggaggtgatccaagatgatgaggtggtggccattctaggaaaaggagacgtgtttggagatgtgttctggaaggaagccacccttgcccagtcctgtgccaatgttagggccttgacctactgtgatctgcatgtgatcaagcgggatgccctgcagaaagtgctggaattctacacggccttctcccattccttctcccggaacctgattctgacgtacaacttgaggaagaggattgtgttccggaagatcagcgatgtgaaacgtgaagaggaagaacgcatgaaacgaaagaatgaggcccccctgatcttgcccccggaccaccctgtccggcgcctcttccagagattccgacagcagaaagaggccaggctggcagctgagagagggggccgggacctggatgacctagatgtggagaagggcaatgtccttacagagcatgcctccgccaaccacagcctcgtgaaggccagcgtggtcaccgtgcgtgagagtcctgccacgcccgtatccttccaggcagcctccacctccggggtgccagaccacgcaaagctacaggcgccagggtccgagtgcctgggccccaaggggggcgggggcgattgtgccaagcgcaaaagctgggcccgcttcaaagatgcttgcgggaagagtgaggactggaacaaggtgtccaaggctgagtcgatggagacacttcccgagaggacaaaagcgtcaggcgaggccacactgaagaagacagactcgtgtgacagtggcatcaccaagagcgacttgcgcctggacaacgtgggtgaggccaggagtccccaggatcggagtcccatcctggcagaggtcaagcattcgttctaccccatccctgagcagacgctgcaggccacagtcctggaggtgaggcacgagctgaaggaggacatcaaggccttaaacgccaaaatgaccaatattgagaaacagctctctgagatactcaggatattaacttccagaagatcctctcagtctcctcaggagttgtttgaaatatcgaggccacagtccccagaatcagagagagacatttttggagccagctga

SEQ ID NO: 100 - protein, 962 aa, Homo sapiens
MTMAGGRRGLVAPQNTFLENIVRRSNDTNFVLGNAQIVDWPIVYSNDGFCKLSGYHRAEVMQKSSTCSFMYGELTDKDTIEKVRQTFENYEMNSFEILMYKKNRTPVWFFVKIAPIRNEQDKVVLFLCTFSDITAFKQPIEDDSCKGWGKFARLTRALTSSRGVLQQLAPSVQKGENVHKHSRLAEVLQLGSDILPQYKQEAPKTPPHIILHYCVFKTTWDWIILILTFYTAILVPYNVSFKTRQNNVAWLVVDSIVDVIFLVDIVLNFHTTFVGPAGEVISDPKLIRMNYLKTWFVIDLLSCLPYDVINAFENVDEGISSLFSSLKVVRLLRLGRVARKLDHYIEYGAAVLVLLVCVFGLAAHWMACIWYSIGDYEIFDEDTKTIRNNSWLYQLAMDIGTPYQFNGSGSGKWEGGPSKNSVYISSLYFTMTSLTSVGFGNIAPSTDIEKIFAVAIMMIGSLLYATIFGNVTTIFQQMYANTNRYHEMLNSVRDFLKLYQVPKGLSERVMDYIVSTWSMSRGIDTEKVLQICPKDMRADICVHLNRKVFKEHPAFRLASDGCLRALAMEFQTVHCAPGDLIYHAGESVDSLCFVVSGSLEVIQDDEVVAILGKGDVFGDVFWKEATLAQSCANVRALTYCDLHVIKRDALQKVLEFYTAFSHSFSRNLILTYNLRKRIVFRKISDVKREEEERMKRKNEAPLILPPDHPVRRLFQRFRQQKEARLAAERGGRDLDDLDVEKGNVLTEHASANHSLVKASVVTVRESPATPVSFQAASTSGVPDHAKLQAPGSECLGPKGGGGDCAKRKSWARFKDACGKSEDWNKVSKAESMETLPERTKASGEATLKKTDSCDSGITKSDLRLDNVGEARSPQDRSPILAEVKHSFYPIPEQTLQATVLEVRHELKEDIKALNAKMTNIEKQLSEILRILTSRRSSQSPQELFEISRPQSPESERDIFGAS

(19) United States
(12) Patent Application Publication
(10) Pub. No.: US 2009/0028793 A1
(43) Pub. Date: Jan. 29, 2009

(54) Vascular Tumor Markers

(75) Inventors: Dario Neri, Buchs (CH); Christoph Rosli, Zurich (CH); Jascha Rybak, Zurich (CH)

Correspondence Address: THE NATH LAW GROUP, 112 South West Street, Alexandria, VA, 22314, US

(73) Assignee: PHILOGEN SPA, Siena (IT)

(21) Appl. No.: 12/223,698

(22) PCT Filed: Feb. 21, 2007
(86) PCT No.: PCT/EP2007/001490

§ 371 Date: Aug. 7, 2008

(30) Foreign Application Priority Data
Feb. 22, 2006 (EP) 06003644.9

(51) Int. Cl. A61K 49/00; C07K 14/00; C12N 15/11; C12N 15/00; C12N 5/06; A61P 31/00; C07K 16/18; A61K 39/395; G01N 33/566; G01N 33/574

(52) U.S. Cl. 424/9.1; 530/350; 536/23.1; 435/320.1; 435/367; 530/387.9; 530/387.3; 424/139.1; 436/501; 435/7.23

(57) ABSTRACT

The present invention relates to a method for identifying neovascular structures in mammalian tissue, wherein said neovascular structures are identified by the detection of at least one specific protein in said tissue. It also relates to a method for identifying diseases or conditions associated with neovascularization, methods for targeting and/or imaging neovascular structures and methods for targeting diseases or conditions associated with neovascularization. Furthermore, the present invention is directed to the use of novel and/or known ligands, preferably antibodies, directed against novel and/or known target proteins for identifying tumor cells in mammalian tissue, preferably mammalian kidney tissue, more preferably mammalian vascular kidney tissue. The present invention also relates to novel ligands, preferably antibodies, fusion proteins comprising said ligands or antibodies, pharmaceutical and diagnostic compositions comprising said ligands, antibodies or fusion proteins, diagnostic and therapeutic methods as well as novel proteins and corresponding polynucleotides, vectors and host cells.

The present invention relates to a method for identifying neovascular structures in mammalian tissue, wherein said neovascular structures are identified by the detection of at least one specific protein in said tissue. It also relates to a method for identifying diseases or conditions associated with neovascularization, methods for targeting and/or imaging neovascular structures and methods for targeting diseases or conditions associated with neovascularization. Furthermore, the present invention is directed to the use of novel and/or known ligands, preferably antibodies, directed against novel and/or known target proteins for identifying tumor cells in mammalian tissue, preferably mammalian kidney tissue, more preferably mammalian vascular kidney tissue. The present invention also relates to novel ligands, preferably antibodies, fusion proteins comprising said ligands or antibodies, pharmaceutical and diagnostic compositions comprising said ligands, antibodies or fusion proteins, diagnostic and therapeutic methods as well as novel proteins and corresponding polynucleotides, vectors and host cells.

### THE PRIOR ART

It is well known in the field of oncology that the growth of solid tumors depends on their capacity to acquire a supporting blood supply. Anti-angiogenics that prevent vascularization at an early stage have been a promising anti-tumor approach. A more recent therapeutic concept is the targeted destruction of established tumor vasculature. Vascular targeting has already been shown to be an effective antitumor strategy in animal models (Neri, D. and Bicknell, R., Nature reviews. Cancer, vol. 5, 436-446, June 2005) and clinical testing for a number of promising compounds has started. Targeting the established tumor vasculature presents an alternative, possibly complementary and certainly wide-ranging therapy.

It has long been known that the endothelium and surrounding stroma in tumors differs from that in normal tissue, but only recently have these differences begun to be characterized at the molecular level. Proteins that are expressed on the endothelial cells or in the surrounding stroma of tumors have been suggested for therapeutic targeting. (Neri and Bicknell, 2005, supra). For example, the toxin ricin was conjugated to high-affinity antibodies directed to a mouse MHC class 11 antigen in solid tumors. The conjugate was injected into mice intravenously and the antibody delivered the ricin specifically to the tumor endothelium, where it was internalized, eliciting cell death with a subsequent collapse of the vasculature and eradication of the solid tumor (Burrows, F. J. and Thorpe, P. E., PNAS USA 90, 8996-9000 (1993). Proteins expressed specifically on the tumor vasculature but not on the vasculature of normal tissues can not only be used for antitumor targeting but also for diagnostic in particular imaging purposes.

For identifying tumor vascular targets most studies are based on in vitro endothelial cell isolates, that are exposed to culture conditions thought to mimic those in normal and tumor tissues and a range of molecular techniques were then employed to identify differentially expressed genes. Although differences in gene expression were apparent, it proved difficult to identify the differentially expressed proteins on the molecular level. Another popular approach has been to raise antibodies to different endothelial structures leading to the identification of new endothelial markers but failed to identify differentially expressed genes, possibly because such proteins are a minor component of the abundant components on the cell surface.

In another recent approach the vasculature has also been targeted in vivo with antibodies directed to vascular antigens. In another recent in vivo targeting approach the present inventors identified accessible antigens in normal organs and in tumors based on the terminal perfusion of tumor-bearing mice with reactive ester derivatives of biotin (Rybak et al., Nat. Methods 2, 291, April 2005).

Tumor-specific vascular targets provide important tumor-diagnostic information and also allow for specifically targeting antitumor compounds. The specific accumulation at the tumor vasculature actively reduces the toxic side effects that are typically associated with the anti-tumor compounds at other locations in the normal tissue and, consequently, allows for the reduction of the concentration of the toxic agents. Moreover, tumor vasculature-specific antitumor agents can be micro-injected in the arterial in-flow of blood into a solid tumor, attach to the vasculature and, thereby, provide a minimum of toxic outflow.

In summary, vascular targets for tumors in general and, in particular, for specific tumors, organ-specific tumors, etc. provide an important tool for the diagnosis and therapy of tumors.

It is the object of the present invention to identify neovascular structures in mammalian tissues, in particular, in mature tissues. Another object is the identification of a disease or condition related to neovascularization in a mammal. A further object is the provision of methods for targeting and/or imaging neovascular structures in mammalian tissues, in particular mature tissues, more particular in tissues affected by a disease. Also, it is the object of the present invention to provide specific tumor targets and uses therefore. Another object underlying the present invention is the provision of kidney-specific tumor targets, in particular vascular kidney tumor targets.

The present invention provides novel polypeptide targets for identifying neovascular structures, in particular neovascular structures in diseases associated with neovascularisation in mammalian tissue such as tumors, macular degeneration, arthritis and atherosclerosis.

Neovasculature structures, as defined herein, are endothelial cells, extracellular matrix, pericytes, other components of the stroma and/or diseased cells in the close proximity of vessels. Such neo-vasculature structures can be found in tumors but also in other angiogenesis-related disorders such as, for example, macular degeneration, arteriosclerosis, rheumatoid arthritis etc.

These new vascular polypeptide targets are selected from the group consisting of:

(1) Periostin [precursor] including isoforms thereof and new splice variants A, B, D, E, (2) putative G-protein coupled receptor 42 including isoforms thereof (3) solute carrier family 2, facilitated glucose transporter member 1, (4) Versican core protein [precursor], (5) CEACAM3 including isoforms thereof, (6) Fibromodulin, (7) Peroxidasin homolog [fragment], (8) probable G-protein coupled receptor 37 [precursor], (9) Protein sidekick-1 [precursor], (10) Alpha1A-voltage-dependent calcium channel, (11) EMILIN2 protein [fragment], (12) Down syndrome critical region protein 8 including isoforms thereof, (13) probable G-protein coupled receptor 113 [precursor], (14) ANXA4 protein [fragment] including isoforms thereof, (15) uromodulin-like 1′ [precursor] including isoforms thereof, (16) scavenger receptor class F member 2 [precursor], (17) Sushi domain-containing protein 2 [precursor], (18) tumor protein, translationally controlled 1, (19) putative G-protein coupled receptor Q8TDUO, (20) hypothetical protein DKFZp686K0275 [fragment], (21) Transmembrane protein TMEM55A, (22) hypothetical protein Q8WYY4, (23) Family with sequence similarity 116, member A, (24) UPF0240 protein C6orf66, (25) CDNA FLJ45811 fis, clone NT2RP7014778, (26) hypothetical protein DKFZp77901248, (27) Beta-ureidopropionase, (28) hypothetical protein DKFZp434F1919 including isoforms thereof, (29) Cysteine-rich with EGF-like domain protein 2 [precursor] including isoforms thereof, (30) UPF0378 family protein KIAA0100 [precursor] (31) potassium voltage-gated channel subfamily H member 1 including isoforms thereof.

Some of the above vascular targets are known proteins, whereas others have been postulated to be proteins from the identification of nucleotide sequences that may code such a protein. A list of (i) the above thirty-one proteins and (ii) the corresponding accession numbers of available amino acid and nucleotide sequences encoding them (Swiss. Prot.) as well as (iii) sequence identification numbers (SEQ ID NOs) relating to the sequences listed further below are provided in the following Table 1.

TABLE 1

| | | Swiss Prot. | SEQ ID No.: |
|---|---|---|---|
| # | Name (synonyms) (comments) | Acc. No. | AA, NA** |
| 1 | Periostin [precursor] (PN, Osteoblast-specific | Q15063 | 1, 2 |
| | factor 2, OSF-2) | | |
| | (Other isoforms:) | | |
| | OTTHUMP00000018269 (Periostin, osteoblast | Q5VSY8 | 3, 4 |
| | specific factor, isoform CRA_a, POSTN protein) | | |
| | OTTHUMP00000018270 | Q5VSY7 | 5, 6 |
| | OTTHUMP00000018271 | Q5VSY6 | 7, 8 |
| | Variant A | | 9, 10 |
| | Variant B | | 11, 12 |
| | Variant D | | 13, 14 |
| | Variant E | | 15, 16 |
| 2 | Putative G-protein coupled receptor 42 | O15529 | 17, 18 |
| | (Other isoforms:) | | |
| | Free fatty acid receptor 3 (G-protein coupled receptor | O14843 | 19, 20 |
| | 41) | | |
| 3 | Solute carrier family 2, facilitated glucose | P11166 | 21, 22 |
| | transporter member 1 (Glucose transporter type | | |
| | 1, erythrocyte/brain; HepG2 glucose transporter) | | |
| 4 | Versican core protein [precursor] | P13611 | 23, 24 |
| | (Large fibroblast proteoglycan, chondroitin sulfate | | |
| | proteoglycan core protein 2, PG-M, glial | | |
| | hyaluronate-binding protein, GHAP) | | |
| 5 | CEACAM3 | Q6UY47 | 25, 26 |
| | (Other isoforms:) | | |
| | Carcinoembryonic antigen-related cell | Q3KPI0 | 27, 28 |
| | adhesion molecule 21 | | |
| | R29124_1 | O75296 | 29, 30 |
| 6 | Fibromodulin (Fibromodulin, isoform CRA_a) | Q8IV47 | 31, 32 |
| 7 | Peroxidasin homolog [fragment] (Melanoma- | Q92626 | 33, 34 |
| | associated antigen MG 50) | | |
| 8 | Probable G-protein coupled receptor 37 | O15354 | 35, 36 |
| | [precursor] (endothelin B receptor-like protein | | |
| | 1, ETBR-LP-1, Parkin-associated endothelin | | |
| | receptor-like receptor, PAELR) | | |
| 9 | Protein sidekick-1 [precursor] (formerly | Q8TEN9 | 37, 38 |
| | identified as FLJ00154 protein) | | |
| 10 | Alpha1A-voltage-dependent calcium channel | Q9NS88 | 39, 40 |
| 11 | EMILIN2 protein [fragment] | Q8N5L1 | 41, 42 |
| 12 | Down syndrome critical region protein 8 | Q96T75 | 43, 44 |
| | (Malignant melanoma-associated protein 1, MMA- | | |
| | 1, MTAG2 protein) | | |
| | (Other isoforms:) | | |
| | Malignant melanoma-associated protein | Q6EXA9 | 45 |
| | (Down syndrome critical region gene 8, isoform | | |
| | CRA_b) | | |
| | Malignant melanoma-associated protein | Q684H4 | 46 |
| | MTAG6 | Q96T75-2 | 47 |
| | MMA-1a | Q96T75-3 | 48 |
| | MMA-1b | Q96T75-4 | 49 |
| 13 | Probable G-protein coupled receptor 113 | Q8IZF5 | 50, 51 |
| | [precursor] (G-protein coupled receptor PGR23) | | |
| 14 | ANXA4 protein [fragment] (Hypothetical protein | Q6LES2 | 52, 53 |
| | ANXA4, proliferation-inducing protein 28) | | |
| | (Other isoforms:) | | |
| | Annexin A4 (Annexin IV, Lipocortin IV, | P09525 | 54, 55 |
| | Endonexin I, Chromobindin-4, Protein II, P32.5, | | |
| | Placental anticoagulant protein II, PAP-II, PP4-X, | | |
| | 35-beta calcimedin, carbohydrate-binding protein | | |
| | P33/P41, P33/41) | | |
| 15 | Uromodulin-like 1 [precursor] (Olfactorin) | Q5DID0 | 56, 57 |
| | (Other isoforms:) | | |
| | Q5DID0-2 | Q5DID0-2 | 58 |
| | UMOLD1S | Q5DID0-3 | 59 |
| | UMOLD1L | Q5DID0-4 | 60 |
| 16 | Scavenger receptor class F member 2 | Q96GP6 | 61, 62 |
| | [precursor] (Scavenger receptor expressed by | | |
| | endothelial cells 2 protein, SREC-II, SRECRP-1) | | |
| 17 | Sushi domain-containing protein 2 [precursor] | Q9UGT4 | 63, 64 |
| | (formerly identified as BK65A6.2) | | |
| 18 | Tumor protein, translationally controlled 1 | Q5W0H4 | 65, 66 |
| 19 | Putative G-protein coupled receptor Q8TDU0 | Q8TDU0 | 67, 68 |
| | (HCG2044627) | | |
| 20 | Hypothetical protein DKFZp686K0275 | Q7Z3A1 | 69, 70 |
| | [fragment] | | |
| 21 | Transmembrane protein TMEM55A | Q8N4L2 | 71, 72 |
| | (hypothetical protein TMEM55A, EC 3.1.3-type II | | |
| | phosphatidylinositol-4,5-biphosphate 4- | | |
| | phosphatase, PtdIns-4,5-P2 4-Ptase II) | | |
| 22 | Hypothetical protein Q8WYY4 | Q8WYY4 | 73, 74 |
| 23 | Family with sequence similaritiy 116, member | Q8IWF6 | 75, 76 |
| | A (Hypothetical protein FLJ34969) | | |
| 24 | UPF0240 protein C6orf66 | Q9P032 | 77, 78 |
| 25 | CDNA FLJ45811 fis, clone NT2RP7014778 | Q6ZS59 | 79, 80 |
| | (formerly identified as hypothetical protein | | |
| | FLJ45811) | | |
| 26 | Hypothetical protein DKFZp779O1248 | Q6AHZ8 | 81, 82 |
| 27 | Beta-ureidopropionase (EC 3.5.1.6 beta-alanine | Q9UBR1 | 83, 84 |
| | synthase, N-carbamoyl-beta-alanine | | |
| | amidohydrolase, BUP-1) | | |
| 28 | Hypothetical protein DKFZp434F1919 | Q9GZU6 | 85, 86 |
| | (Hypothetical protein MDS025, Coiled-coil domain | | |
| | containing 90B, CUA003) | | |
| | (Other isoform:) | | |
| | MDS011 (Coiled-coil domain containing 90B, | Q9GZT6 | 87, 88 |
| | MDS025) | | |
| 29 | Cysteine-rich with EGF-like domain protein 2 | Q6UXH1 | 89, 90 |
| | [precursor] (CRELD2 protein) (Q9BU47, | | |
| | Q86UC0 and Q6UXH1 were formerly separate | | |
| | entries in Swiss Prot.) | | |
| | (Other isoforms:) | | |
| | Alpha | Q6UXH1-2 | 91 |
| | Beta | Q6UXH1-3 | 92 |
| | Gamma | Q6UXH1-4 | 93 |
| | Epsilon | Q6UXH1-5 | 94 |
| | Zeta | Q6UXH1-6 | 95 |
| 30 | UPF0378 family protein KIAA0100 [precursor] | Q5H9T4 | 96, 97 |
| | (breast cancer overexpressed gene 1 protein, | | |
| | antigen MIaa-22) (*formerly identified as | | |
| | hypothetical protein DKFZp686M0843) | | |
| 31 | Potassium voltage-gated channel subfamily H | O95259 | 98, 99 |
| | member 1 (Voltage-gated potassium channel | | |
| | subunit Kv10.1, Ether-a-go-go potassium channel | | |
| | 1, hEAG1. h-eag) (*formerly identified as | | |
| | Potassium voltage-gated channel subfamily H | | |
| | member 1 (KCNH1)) | | |
| | (Other isoforms:) | | |
| | hEAG | O95259-2 | 100 |

*previously identified under this name in the priority document
**AA, NA = amino acid sequence, nucleic acid sequence

The terms “[fragment]” and “[precursor]” in the context of the above listed proteins are part of their actual name in the database entry of the respective protein and are not to be considered as limiting in any way to the scope of the invention.

Furthermore, it is common knowledge in the art that database entries sometimes contain minor sequencing errors and may be subject to revisions and changes. In addition, proteins can be subject to posttranslational modifications and differential splicing. Therefore, it is preferred that any reference herein to any of the above thirty-one vascular tumor marker proteins also refers to any sequence fragments, splice variants, posttranslationally modified variants and/or sequences thereof containing further extensions as well as other synonyms of the above listed proteins. More preferred herein, reference to the above vascular tumor marker proteins encompass variants thereof that can be identified by mass spectrometric analysis because they contain the peptide sequences identified in the table at the end of the examples in bold letters.

The above new vascular polypeptide targets were identified by an ex vivo vascular perfusion of surgically removed kidneys with a biotinylation reagent that labels vascular accessible primary amine-containing structures with biotin. The isolation, characterization and subsequent comparison of the many biotin-labelled amine structures in the vasculature of kidneys with and without tumors eventually led to the identification of the above vascular tumor targets. For details of the identification procedure see the example 1 below.

The above vascular targets now allow for the preparation of vascular target-specific ligands. Ligands for use according to the present invention include antibodies, antibody fragments or functional derivatives thereof as well as antibody-like binding molecules, peptides, small organic molecules, aptamers and other binding molecules as described below having a binding affinity to one of the above-listed proteins in table 1.

These vascular target-specific ligands are useful for the methods and uses of the present invention.

In a first aspect the present invention relates to a method for identifying neovascular structures in mammalian tissue, wherein said neovascular structures are identified by the detection of at least one protein in said tissue, the at least one protein being selected from the proteins identified in Table 1 above. Preferably, the mammalian tissue is mature mammalian tissue, more preferably human mature tissue, most preferably kidney tissue.

The term “mature tissue”, as it is used herein, is understood to mean fully differentiated tissue from born mammals, preferably adult mammals and specifically excludes prenatal tissue.

Another aspect of the present invention provides for a method for identifying a disease or condition in a mammal selected from the group consisting of tumors, macular degeneration, arthritis and/or atherosclerosis, wherein said disease or condition is identified by the detection of at least one protein within and/or in the close proximity of mammalian tissue of interest, said at least one protein being selected from the proteins identified in Table 1 above. Preferably, said disease is a tumor, more preferably a human tumor, most preferably a human kidney tumor.

The present invention also encompasses a method for targeting and/or imaging neovascular structures in mammalian tissue, wherein said neovascular structures are targeted and/or imaged by a ligand having specific binding affinity to at least one protein in said neovascular structure, the at least one protein being selected from the proteins identified in Table 1 above. Preferably, said mammalian tissue is mature mammalian tissue, more preferably human mature tissue, most preferably kidney tissue.

A further aspect of the invention is directed to a method for targeting and/or imaging a tissue affected by a disease or condition in a mammal selected from the group consisting of tumors, macular degeneration, arthritis and/or atherosclerosis, wherein said disease or condition is targeted and/or imaged by a ligand having a specific binding affinity to at least one protein within and/or in the close proximity of the mammalian tissue of interest, said at least one protein being selected from the proteins identified in Table 1 above. Preferably, said disease is a tumor, preferably a kidney tumor, more preferably a human kidney tumor.

While monoclonal antibodies and their derivatives are still the preferred binding molecules/ligands for pharmaceutical biotechnological applications, other classes of binding molecules/ligands with antibody-like binding properties have increasingly been used as alternatives to antibodies for many applications. Such functional analogues include aptamers (Brody E N, Gold L., Aptamers as therapeutic and diagnostic agents. J. Biotechnol. 2000 Mar., 74(1):5-13. Review), small globular proteins engineered (e.g., by mutagenesis of loops) to recognize cognate antigens (e.g., anticalins, affibodies, ankyrin repeats, etc. [Binz H K, Amstutz P, Pluckthun A; Engineering novel binding proteins from nonimmunoglobulin domains. Nat. Biotechnol. 2005 Oct., 23(10):1257-68. Review.]). Globular proteins having antibody-like proteins can be derived from large libraries of mutants, e.g. be panned from large phage display libraries and can be isolated in analogy to regular antibodies. Also, antibody-like binding proteins can be obtained by combinatorial mutagenesis of surfaces-exposed residues in globular proteins. Moreover, low molecular weight synthetic organic molecules can be used as vascular tumor targeting agents, provided that they have sufficient binding affinity and specificity for the antigen as well as suitable pharmacokinetic properties.

Therefore, in another aspect, the present invention relates to the use of at least one ligand, preferably at least one antibody, fragment or functional derivative thereof, having specific binding affinity to a protein selected from Table 1 for identifying neovascular structures, preferably for identifying tumors, in mammalian tissue.

In a preferred embodiment the at least one ligand, preferably an antibody, fragment or functional derivative thereof, has specific binding affinity to a protein selected from: 1A, 1B, 1D, 1E, 2, 5, 7-13, 15-17, 19-23, 25-30.

The proteins in table 1 above and the preferred proteins listed directly above were specifically identified in neo-vasculature structures of human tumor tissue.

Therefore, in a more preferred embodiment the present invention relates to the use according to the present invention for identifying tumors in human tissue.

The proteins of table 1 were identified in neo-vasculature structures of human kidney tumor tissue. Therefore, in a further more preferred embodiment, the present invention relates to the use according to the invention for identifying neovascular structures, in particular for identifying tumors, in mammalian kidney tissue, preferably human kidney tissue.

Most preferred the proteins for identifying neo-vasculature structures in mammalian kidney tissue, preferably human kidney tissue, are selected from the group consisting of: 1, 2, 4-13, 15-31.

All of the proteins in table 1 were specifically identified in neo-vasculature structures of human kidney tumors. They represent specific targets in kidneys that are accessible from the blood stream. Therefore, in a most preferred embodiment the present invention relates to the use of at least one ligand, preferably at least one antibody, fragment or functional derivative thereof, having specific binding affinity to a protein selected from Table 1 for identifying neovascular structures, in particular tumors, in mammalian vascular kidney tissue, preferably human vascular kidney tissue.

The above uses according to the present invention provide for tumor diagnostic methods in vitro and in vivo. For example, ligands such as antibodies having specific binding affinity to at least one of the neo-vasculature tumor targets of table 1 may be contacted with cells, tissue and/or organs under conditions that allow for the binding of said ligands, preferably antibodies, to their corresponding target protein. Ligand-bound, preferably antibody-bound cells, tissue and/or organs are then identified as tumor or tumor-associated cells, tissue and/or organs. The identification of the bound ligands/antibodies may be performed by any of the many routine techniques available to the skilled person that have become routine in the art such as, e.g. secondary antibodies or the identification of markers conjugated to the ligands/antibodies such as radiolabels and chemical labels. The step of contacting the ligands/antibodies and/or the identification of ligand/antibody-bound tumor cells, tissue and/or organs may be performed in vivo, e.g. by a radio-imaging method. However, the contacting step may also be performed in vivo in a mammal, subsequently isolating cells, tissue and/or the organ of interest and identifying antibody-bound tumor cells in vitro/ex vivo.

Preferably, said ligands/antibodies are used to identify tumor cells in vitro only. The term “in vitro” is meant to indicate that the use of said ligands/antibodies according to the invention is limited to methods that are not practiced on the human or animal body and therefore do not violate Art. 52(4) EPC.

In another aspect the present invention is also directed to a ligand, preferably an antibody, fragment or functional derivative thereof, having specific binding affinity to a protein selected from the group consisting of the proteins in table 1 above.

Preferably, the ligand, preferably an antibody, fragment or functional derivative according to the invention has a specific binding affinity to a protein selected from the group consisting of:

(1) Periostin splice variants A, B, D, E, (5) CEACAM3 including isoforms thereof, (7) Peroxidasin homolog [fragment], (9) Protein sidekick-1 [precursor], (12) Down syndrome critical region protein 8 including isoforms thereof, (13) probable G-protein coupled receptor 113 [precursor], (15) uromodulin-like 1 [precursor] including isoforms thereof, (16) scavenger receptor class F member 2 [precursor], (17) Sushi domain-containing protein 2 [precursor], (19) putative G-protein coupled receptor Q8TDUO, (20) hypothetical protein DKFZp686K0275 [fragment], (21) Transmembrane protein TMEM55A, (22) hypothetical protein Q8WYY4, (23) Family with sequence similarity 116, member A, (25) cDNA FLJ45811 fis, clone NT2RP7014778, (28) hypothetical protein DKFZp434F1919 including isoforms thereof, (30) UPF0378 family protein KIAA0100 [precursor].

The term “specific binding affinity” as it is used herein is to be understood to mean that the ligand/antibody specifically binds to the target protein with significant affinity and not to other proteins with significant affinity that are also located in the same environment, i.e. assay system, diagnostic or therapeutic setting in vivo or in vitro, in an organ, e.g. kidney, and under the same conditions, e.g. pH, temperature, buffer, etc. In general, a binding specificity is tested by performing a binding assay with a specific target molecule and with a large number of non-related substances. Furthermore, functional tests, immunohistochemistry and other procedures can be used to assess the binding specificity of a certain ligand (e.g. an antibody).

For many bioassays (e.g. ELISA) based on ligands, e.g. antibodies or globular proteins, capable of specific binding, a dissociation constant of 1 micromolar or lower is required to yield detectable binding signals which are often associated with a specific binding mode. Preferably, the ligands/antibodies for use in the present invention have a specific binding affinity corresponding to a dissociation constant of less than about 5, preferably about 1 or less micromolar (μM), more preferably about 0.1 μM or less, most preferably about 1 nM or less or even 1 μM or less.

Ligands such as antibodies and fragments according to the invention are routinely available by hybridoma technology (Kohler, G. and Milstein, C. Nature 256, 495-497, 1975), antibody phage display (Winter et al., Annu. Rev. Immunol. 12, 433-455, 1994), ribosome display (Schaffitzel et al., J. Immunol. Methods, 231, 119-135, 1999) and iterative colony filter screening (Giovannoni et al., Nucleic Acids Res. 29, E27, 2001) once the target antigen is available. Typical proteases for fragmenting antibodies into functional products are well-known. Other fragmentation techniques can be used as well as long as the resulting fragment has a specific high affinity and, preferably, a dissociation constant in the micromolar to picomolar range.

The vascular tumor targeting performance of antibody fragments in scFv format has been shown to crucially depend (at least for a micromolar to picomolar dissociation constant) on the affinity of the antibody to the target. For example, the high affinity antibody fragment scFv(L19), specific to the EDB domain of fibronectin, a marker of angiogenesis, was shown to target tumor neo-vasculature more efficiently than the parental antibody fragment scFv(E1), with a lower affinity for the antigen [Viti F, Tarli L, Giovannoni L, Zardi L, Neri D.; Increased binding affinity and valence of recombinant antibody fragments lead to improved targeting of tumoral angiogenesis. Cancer Res. 1999 Jan. 15; 59(2):347-52.]. In certain cases, binding avidity (e.g., associated with certain homobivalent antibody formats) can compensate for a moderate monomeric binding affinity [Nielsen U B, Adams G P, Weiner L M, Marks J D; Targeting of bivalent anti-ErbB2 diabody antibody fragments to tumor cells is independent of the intrinsic antibody affinity. Cancer Res. 2000 Nov. 15, 60(22):6434-40.].

A very convenient antibody fragment for targeting applications is the single-chain Fv fragment, in which a variable heavy and a variable light domain are joined together by a polypeptide linker. Other antibody fragments for vascular targeting applications include Fab fragments, Fab₂ fragments, miniantibodies (also called small immune proteins), tandem scFv-scFv fusions, as well as scFv fusions with suitable domains (e.g. with the Fc portion of an immunoglobulin). For a review on certain antibody formats, please see Holliger P, Hudson P J.; Engineered antibody fragments and the rise of single domains. Nat. Biotechnol. 2005 Sep., 23(9):1126-36. Review.

The term “functional derivative” of an antibody for use in the present invention is meant to include any antibody or fragment thereof that has been chemically modified in its amino acid sequence, e.g. by addition, substitution and/or deletion of amino acid residue(s) and/or has been chemically modified in at least one of its atoms and/or functional chemical groups, e.g. by additions, deletions, rearrangement, oxidation, reduction, etc. as long as the derivative has substantially the same binding affinity to the corresponding antigen from table 1 and, preferably, has a dissociation constant in the micro-, nano- or picomolar range. A most preferred derivative of the antibodies for use in the present invention is an antibody fusion protein that will be defined in more detail below.

In a preferred embodiment, the antibody, fragment or functional derivative thereof according to the invention is one that is selected from the group consisting of polyclonal antibodies, monoclonal antibodies, chimeric antibodies, humanized antibodies, CDR-grafted antibodies, Fv-fragments, Fab-fragments and Fab₂-fragments and antibody-like binding proteins.

Next to said ligands, preferably antibodies, fragments and derivatives a further aspect of the present invention is directed to fusion proteins comprising a ligand, preferably an antibody, fragment or functional derivative thereof, according to the present invention.

The term “fusion protein” as it is used in the context of the present invention is meant to encompass all conjugates, wherein a ligand/antibody, fragment or functional derivative according to the present invention is somehow bound to any further component such as, e.g. polypeptide, signal factor, e.g. interleukin, protein, sugar moiety, nucleotide, small biologically active molecule, toxin, label, radiolabel, etc. by, e.g. covalent and/or non-covalent, e.g. ionic bonds.

Preferably, the fusion protein according to the invention additionally comprises a component having anti-tumor activity. This will greatly facilitate the selectivity as well as the specificity of the anti-tumor compound, thus, allowing for reducing the effective amount thereof to be administered to a patient in need thereof as well as for reducing the toxic side effects associated with said compound.

Intact monoclonal antibodies represent a well-established class of pharmaceuticals with a broad therapeutic potential for various indications. The constant portion of the antibody often contributes to the therapeutic potential and glycosylation can influence bioactivity (Li H, Sethuraman N, Stadheim T A, Zha D, Prinz B, Ballew N, Bobrowicz P, Choi B K, Cook W J, Cukan M, Houston-Cummings N R, Davidson R, Gong B, Hamilton S R, Hoopes J P, Jiang Y, Kim N, Mansfield R, Nett J H, Rios S, Strawbridge R, Wildt S, Gerngross T U; Optimization of humanized IgGs in glycoengineered Pichia pastoris. Nat. Biotechnol. January 2006). Furthermore, a number of vascular targeting antibody derivatives can be considered for pharmaceutical intervention. They include antibody conjugates with radionuclides, photosensitizers, liposomes and drugs, as well as antibody-based fusion proteins with pro-coagulant agents, cytokines, chemokines, toxins, Fc fusions, as well as bispecific antibodies.

More preferably, the fusion protein according to the invention comprises a component having anti-tumor activity that is selected from the group consisting of intact antibodies, Fc-containing antibody fragments or Fc-functional derivatives thereof, radionucleotides, photosensitizers, liposomes, drugs, pro-coagulatory agents, cytokines, chemokines, toxins as well as bispecific antibodies.

It is well established that ligands such as antibody derivatives can contribute to the diagnosis and/or molecular imaging of a disease. The most established avenues for the macroscopic imaging of ligand/antibody localization in vivo include the use of radiolabeled ligands/antibodies (e.g., for PET or SPECT applications) and the use of ligands/antibodies labeled with infrared fluorophores (e.g., for superficial fluorescence imaging, for endoscopic imaging, for diffuse optical tomography, etc.). Moreover, ligand/antibody-microbubble conjugates (to be used as contrast agents in ultrasound-based imaging procedures; Joseph S, Olbrich C, Kirsch J, Hasbach M, Briel A, Schirner M.; A real-time in vitro assay for studying functional characteristics of target-specific ultrasound contrast agents. Pharm Res. 2004 Jun., 21(6):920-6.) and/or ligand/antibody-conjugates for enhancing MRI imaging (Kiessling F, Heilmann M, Lammers T, Ulbrich K, Subr V, Peschke P, Waengler B, Mier W, Schrenk H H, Bock M, Schad L, Semmler W. Synthesis and characterization of HE-24.8: a polymeric contrast agent for magnetic resonance angiography. Bioconjug Chem. 2006 January-February; 17(1):42-51.) can also be used.

In another more preferred embodiment, the fusion protein according to the present invention comprises a component having diagnostic activity, i.e. allowing for the selective identification of the antibody component in vivo and/or ex vivo.

Preferably, the component having diagnostic activity is selected from the group consisting of radiolabels, fluorophores, biotin, chelated metals or metal compounds, and microbubbles.

The fusion protein of the present invention having an anti-tumor component is useful for preparing a medicament that is efficiently targeted to the vasculature of tumors, preferably to kidney tumors. Therefore, a further aspect of the present invention relates to the use of a fusion protein according to the invention for preparing a medicament for the treatment of cancer in a mammal, preferably in a human.

Preferably, said medicament is for the treatment of kidney cancer, preferably human kidney cancer.

The fusion protein of the present invention comprising a component having diagnostic activity is useful for preparing a medicament that is efficiently targeted to the vasculature of tumors, preferably to kidney tumors. Therefore, a further aspect of the present invention relates to the use of a fusion protein according to the invention for preparing a diagnostic composition for the identification of tumors in a mammal, preferably a human.

Preferably, said diagnostic composition is for the identification of tumors in a mammalian kidney, preferably a human kidney.

Another aspect of the present invention relates to a pharmaceutical composition, comprising a ligand, preferably an antibody, fragment or derivative thereof, or a fusion protein according to the present invention and a pharmaceutically acceptable carrier and/or diluent.

A further aspect of the present invention relates to a diagnostic composition, comprising a ligand, preferably an antibody, fragment or derivative thereof, or a fusion protein according to the invention.

Another aspect of the present invention is directed to a method for identifying tumors in mammalian tissue, preferably human tissue, comprising:
(i) contacting a ligand, preferably an antibody, fragment or functional derivative thereof and/or a fusion protein comprising a ligand, preferably an antibody, fragment or functional derivative thereof, having specific binding affinity to at least one protein selected from the group listed above in table 1.
   with mammalian tissue of interest, preferably human tissue of interest, in vivo and/or ex vivo under conditions that allow for the specific binding of said ligand and/or fusion protein to at least one of said proteins 1 to 31, and
(ii) identifying specifically bound ligand and/or fusion protein.

Preferably, the at least one tissue protein is selected from the group consisting of: (All numbers according to table 1) 1A, 1B, 1D, 1E, 2, 5, 7-13, 15-17, 19-23, 25-30.

More preferably, the at least one tissue protein is selected from the group consisting of: 1-2, 4-13, 15-31, and the mammalian tissue of interest is kidney tissue.

Also, it is preferred that the mammalian tissue of interest is vascular kidney tissue, preferably human vascular kidney tissue.

More preferably, said steps (i) and/or (ii) of the method according to the invention are performed ex vivo, i.e. in vitro.

Most preferred, the method according to the present invention is a method of imaging neovascular structures, in particular a method of imaging tumor cells, preferably kidney tumors, more preferably vascular kidney tumors in vivo.

The present invention also identifies a number of novel proteins that have utility as novel tumor markers, novel kidney-specific tumor markers, novel and specific markers of the vasculature of kidney tumors, and that can be used as antigens for providing selective and high affinity antibodies as diagnostic and therapeutic means.

### Four Novel Splice Variants of Periostin

Periostin is a 90 kDa protein initially identified as osteoblast-specific factor-2 (OSF-2, also called PN), secreted by osteoblasts (Takeshita et al., Biochem J, 294 (Pt 1), 271-8, 1993). Tai and colleagues produced a monoclonal anti-periostin antibody by hybridoma technology and detected, by Western blotting, expression of the human periostin protein in the adrenal glands, lung, thyroid, uterus, vagina, ovary, testis, prostate, and in the gastrointestinal tract, with a preferential expression in the stomach and colorectum, while lower levels were noted in the small intestine and esophagus (Tai et al., Carcinogenesis, 26, 908-15, 2005).

There have been observations of periostin being associated in a number of cancers. However, Periostin has not been associated with kidney tumors in the prior art.

For human periostin, Takeshita and colleagues (Takeshita, Kikuno et al., Biochem J. 294 (Pt 1), 271-8, 1993) have reported that five alternative spliced transcripts can be produced, and that all splicing events of periostin occur within the C-terminal region. The same group has found in the mouse four possible isoforms of periostin generated by a combination of six different cassettes (Horiuchi, Amizuka et al., J. Bone Miner Res., 14, 123949, 1999). The function of the isoforms have not yet been elucidated. Litvin and colleagues identified another isoform of mouse periostin and termed it periostin-like-factor (PLF) (Litvin et al., J. Cell Biochem., 92, 1044-61, 2004). A sequence analysis of the full-length PLF cDNA and predicted aa sequence showed that it most resembles Horuichi's isoform 3 of mouse periostin (Litvin, Selim et al., J Cell Biochem, 92, 1044-61, 2004).

The present invention demonstrates for the first time that periostin is a protein overexpressed in kidney cancer. Therefore, it can be used as an excellent kidney tumor marker readily accessible from the bloodstream and, thus, is also a useful target for ligand-based tumor targeting strategies.

An immunohistochemical analysis with an anti-periostin antibody further proved that periostin was highly overexpressed in the tumor stroma of renal clear cell carcinoma compared to normal kidney tissue.

A PCR amplification of the C-terminal region of periostin revealed at least eight different splice variants. In public protein databases (Expasy and NCBI), only four different isoforms of human periostin are described (the full-length form and the three splice isoforms Q5VSY8, Q5VSY7 and Q5VSY6). Next to all published isoforms, four new isoforms, termed isoforms A, B, D and E (see SEQ ID NO: 9, 11, 13, 15, respectively) were identified. The corresponding analysis showed a different distribution of isoform transcripts in the various tissues. It was found that the transcripts of periostin are only weakly expressed (or barely detectable) in normal adult kidney cDNA, but could be amplified from the clear cell carcinoma specimen in isoforms of different length. This finding is compatible with the identification of periostin in a proteomic analysis in the tumor tissue only. Fetal kidney was also positive for periostin, but the distribution of the isoforms was different from that registered in all the tumor tissues examined. Expression of periostin transcripts can also be seen in normal adult brain and liver. However, the distribution of isoforms of periostin in brain and liver cDNA libraries showed differences between tumor, fetal and normal adult specimens. The smallest detected transcript of periostin was predominantly expressed in tumor specimens, being present in at least four different kidney and liver tumors, but undetectable in normal specimens and only barely detectable in normal adult brain and fetal kidney.

Surprisingly, a mass spectrometric analysis revealed three peptides EIPVTVYKPIIKK, EIPVTVYRPTLTK and IITGPEIK, which are isoform-specific, because they encompass junctions of two exons which only exist in certain isoforms.

It is expected that ligands, preferably antibodies directed/raised against “junction peptides” existing only in the novel periostin isoforms specifically expressed in tumors (or even a particular type of tumor) will provide a very powerful tool for selective targeting and destruction of these tumors.

In one aspect, the present invention is directed to a periostin splice variant protein, fragment or functional derivative, comprising a peptide having the amino acid EIPVTVYGPEIK.

Preferably, this aspect of the present invention relates to a periostin splice variant protein A, B, D or E having an amino acid sequence selected from SEQ ID NOs: 9, 11, 13, 15, respectively, a fragment or a functional derivative thereof, wherein the amino acid sequence of the fragment or functional derivative thereof comprises at least 20, preferably at least 30, more preferably at least 50 amino acids, and most preferably at least 75 amino acids, and

a) wherein the fragment or functional derivative of SEQ ID NO: 9 has an amino acid sequence reflecting deletions in SEQ ID NO: 1 in positions 670-756, or in positions 670-756 and 783-810;
b) wherein the fragment or functional derivative of SEQ ID NO: 11 has an amino acid sequence reflecting deletions in SEQ ID NO: 1 in positions 670-726 and 784-810 or in positions 784-810; preferably, this splice variant comprises a peptide having the amino acid sequence EIPVTVYGPEIK.
c) wherein the fragment or functional derivative of SEQ ID NO: 13 has an amino acid sequence reflecting a deletion in SEQ ID NO: 1 in positions 670-756;
d) wherein the fragment or functional derivative of SEQ ID NO: 15 has an amino acid sequence comprising the amino acid valine in position 421 in SEQ ID NO: 15 and an amino acid sequence reflecting a deletion in SEQ ID NO: 1 in positions 671-697.

A preferred embodiment relates to a periostin splice variant according to the invention having at least 80, preferably 85, more preferably 90, most preferably at least 95 or 98% amino acid sequence identity to the above protein, fragment or functional derivative according to the invention, wherein the sequence is not a sequence of any one of SEQ ID NO: 1, 3, 5 and 7.

Furthermore, the present invention relates to a polynucleotide encoding an above described protein, fragment or functional derivative of the present invention of any one of the 4 novel splice variants.

A functional derivative of a protein according to the invention is meant to encompass any amino acid sequence- and/or chemical derivative, that has substantially sufficient accessible amino acid residues to establish the same binding affinity to an antibody that has an affinity to the original protein. Preferably, the functional derivative is one that has deletions (including N-terminal or C-terminal truncations), additions and/or substitutions, more preferably conservative amino acid substitutions.

A fragment or a functional derivative according to the present invention has at least 10, at least 20, at least 30, at least 40, at least 50, at least 75 or at least 100 amino acids of the original full length protein.

For determining the sequence identity among polypeptides, the skilled person can revert to a number of standard algorithms known to those of skill in the art.

Preferably, the BLAST programs at http://www.expasy.org/tools/blast/ and http://www.ncbi.nlm.nih.gov/BLAST/Blast.cgi?CMD=Web&LAYOUT=TwoWindow s&AUTO_FORMAT=Semiauto&ALIGNMENTS=250&ALIGNMENT_VIEW=Pairwi se&CDD_SEARCH=on&CLIENT=web&DATABASE=nr&DESCRIPTIONS=500& ENTREZ_QUERY=%28none %29&EXPECT=10&FILTER=L&FORMAT_OBJECT=Alignment&FORMAT_TYPE=HTML&I_THRESH=0.005&MATRIX_NAME=BLO SUM62&NCBI_GI=on&PAGE=Proteins&PROGRAM=blastp&SERVICE=plain&S ET_DEFAULTS.x=41 &SET_DEFAULTS.y=5&SHOW_OVERVIEW=on&END_OF_HTTPGET=Yes&SHOW_LINKOUT=yes&GET_SEQUENCE=yes, more preferably with the default settings, are used to identify the amino acid sequence identity of a protein, protein fragment or protein derivative of the present invention.

In some instances the present invention also provides novel polynucleotides encoding the proteins, fragments or functional derivatives thereof of the present invention characterized in that they have the ability to hybridize to a specifically referenced nucleic acid sequence under stringent conditions. Next to common and/or standard protocols in the prior art for determining the ability to hybridize to a specifically referenced nucleic acid sequence under stringent conditions, it is preferred to analyse and determine the ability to hybridize to a specifically referenced nucleic acid sequence under stringent conditions by comparing the nucleotide sequences of the two proteins, which may be found in gene databases (e.g. http://www.ncbi.nlm.nih.gov/entrez/query.fcgi?db=nucleotide) with alignment tools (e.g., http://www.ncbi.nim.nih.gov/blast/Blast.cgi?CMD=Web&LAYOUT=TwoWindows&AUTO_FORMAT=Semiauto&PAGE=Nucleotides&NCBI_GI=yes &FILTER=L&HITLIST SIZE=100&SHOW_OVERVIEW=yes&AUTO_FORMAT=y es&SHOW_LINKOUT=yes).

The term “polynucleotide encoding a protein” as it is used in the context of the present invention is meant to include allelic variations and redundancies in the genetic code.

Furthermore, the present invention provides new proteins, fragments and derivatives thereof, as well as nucleotides encoding them in accordance with any of the claims 40 to 93.

More specifically the present invention provides proteins 5 (3×), 7, 9, 12 (6×), 13, 15 (4×), 16, 19-23, 25, 28 (2×), 30, 31 (2×) having an amino acid sequence as indicated in the corresponding SEQ ID NO: (see Table 1 for assignment), fragments or functional derivatives thereof.

Furthermore, in a preferred embodiment the present invention relates to proteins, fragments or functional derivatives thereof having at least 70, preferably 80, more preferably 90, most preferably at least 95% amino acid sequence identity to the above proteins 5 (3×), 7, 9, 12 (6×), 13, 15 (4×), 16, 19-23, 25, 28 (2×), 30, 31 (2×), fragments or functional derivatives thereof.

Moreover, the present invention is directed to polynucleotides encoding any one of the above proteins 5 (3×), 7, 9, 12 (6×), 13, 15 (4×), 16, 19-23, 25, 28 (2×), 30, 31 (2×), fragments or functional derivatives thereof, mentioned above in accordance with the present invention, having the ability to hybridize to the corresponding nucleic acid sequence (see Table 1 for assignment) encoding the complete protein under stringent conditions.

Also, the present invention encompasses vectors comprising the polynucleotides encoding proteins, fragments and functional derivatives according to the present invention as well as host cells comprising said proteins, fragments and functional derivatives and/or vectors of the present invention.

Last but not least, a further aspect of the present invention is directed to methods for recombinantly producing proteins, fragments and functional derivatives of the present invention employing polynucleotides, vectors and/or host cells according to the present invention.

In the following the target proteins that have demonstrated their utility as tumor targets, in particular vascular kidney tumor targets according to the present invention are briefly discussed.

### Protein #2

The identified peptide can be derived from one or both of two protein isoforms (Table 1: 2): Free fatty acid receptor 3 (O14843) and/or putative G-protein coupled receptor 42 (O15529), respectively.

Within family A of the G protein-coupled receptor gene superfamily (also classified as family 1), there is a phylogenetically related group of −90 receptors that respond to an unusually wide variety of ligand types, considering the relatively close similarity of their primary sequences (Bockaert and Pin, Embo J, 18, 1723-9, 1999).

Free fatty acid receptor 3 was detected in adipose in all three published studies on this receptor (Brown et al., J. Biol. Chem., 278, 11312-9, 2003; Le Poul et al., J. Biol. Chem., 278, 25481-9, 2003; Xiong et al., Proc. Natl. Acad. Sci. USA, 101, 1045-50, 2004). So far, an antibody for discriminating between GPR41 and GPR42 is unknown.

An expression or even overexpression of either of these proteins in tumors has not yet been reported.

### Protein #3

Solute carrier family 2, facilitated glucose transporter member 1 (SLC2A1) (=Glucose transporter type 1, erythrocyte/brain (GLUT1)) (P11166)

Increased glucose uptake is one of the major metabolic changes found in malignant tissue. This uptake is mediated by glucose transporter (Glut) proteins, which are membrane proteins responsible for the transport of glucose across cellular membranes. These human glucose transporters have a distinct tissue distribution and contribute to the disposal of glucose under various conditions (Pessin and Bell, Annu Rev Physiol, 54, 911-30, 1992). A family of seven glucose transporters has been cloned. Among these, Glut1 (which is also called solute carrier family 2, facilitated glucose transporter member 1 (SLC2A1)) is expressed in erythrocytes, the blood-brain barrier, the perineurium of peripheral nerves and the placenta (Froehner et al., J. Neurocytol., 17, 173-8, 1988; Pardridge et al., J Biol Chem, 265, 18035-40, 1990; Pessin and Bell, Annu. Rev. Physiol., 54, 911-30, 1992; Takata et al., Cell Tissue Res, 267, 407-12, 1992). Glut1 has been associated with a number of tumors in the prior art. Glut1 was also shown by immunohistochemistry to be expressed in kidney cancer (Nagase et al., J Urol, 153, 798-801, 1995; North et al., Clin Neuropathol, 19, 131-7, 2000).

The present invention demonstrates for the first time, that Glut1 is a protein readily accessible from the bloodstream, indicating this tumor marker to be a useful target for ligand-based tumor targeting strategies.

### Protein #4

Versican core protein [precursor] (13611) is a large extracellular matrix proteoglycan that is present in a variety of tissues and plays a role in the regulation of cell adhesion and survival, cell proliferation, cell migration and extracellular matrix assembly (Wight, Curr Opin Cell Biol, 14, 617-23, 2002). In addition, there is evidence that versican is overexpressed in angiogenesis and in tumors.

The present invention surprisingly demonstrates the expression of the versican core protein in kidney cancer, indicating this tumor marker to be a useful target for ligand-based tumor targeting strategies.

### Protein #5

Surprisingly, the peptide SDPLKLTVK was identified in tumor only but not in normal kidney samples. This peptide is part of three different sequence entries in the SwissProt database: CEACAM3 with 292 amino acid residues (Q6UY47), carcinoembryonic antigen-related cell adhesion molecule (gene name: CEACAM21) with 293 amino acid residues (Q3 KPI0), and R29124_{—}1 with 235 amino acid residues (075296). The existence of these proteins was postulated based on DNA sequence analysis, but was never experimentally proven. The three sequences share more than 98% identity with a maximum of 3 mismatches. These data indicates that the three sequences either belong to the same protein and the differences are due to sequencing errors, or that the sequences correspond to different isoforms of the same protein.

The sequences exhibit significant similarity to other proteins of the CEACAM family, a subgroup of the human carcinoembryonic Ag (CEA) protein family (Beauchemin et al., Exp Cell Res, 252, 243-9, 1999).

While a number of CEACAM have been studied on the protein level, this seems not to be the case for the proteins identified in this invention. The highest similarity to a CEACAM which has actually been studied on the protein level is to biliary glycoprotein precursor (CEACAM1) and is only maximal 44%. Furthermore, the CEACAM1 sequence does not contain the identified peptide.

The present invention surprisingly identifies the peptide SDPLKLTVK and, thus, proves the existence of a protein with a sequence predicted in the database entries Q6UY47, Q3 KPI0, and O75296. So far, there are no antibodies available which specifically recognize this protein.

In addition, the present invention identifies the above-mentioned peptide in tumors but not in normal kidney, thus, indicating said protein as a novel marker overexpressed in human tumors, more preferably as a human kidney tumor marker, most preferably as a human kidney tumor marker readily accessible from the bloodstream and, thus, useful as a target for ligand-based tumor targeting applications.

### Protein #6

In the present invention the peptide YLPFVPSR was identified as part of the protein fibromodulin (Q81V47). Fibromodulin was first described as a 59-kDa protein (Heinegard et al., J Biol Chem, 261, 13866-72, 1986) that interacts with collagen types 1 and 11 (Hedbom and Heinegard, J Biol Chem, 264, 6898-905, 1989) and is present on collagen fibers in cartilage (Hedlund et al., Matrix Biol., 14, 227-32, 1994). Fibromodulin is thought to play an important role in collagen fiber formation as shown by the observation that FM null mice form abnormal collagen fibrils in tendons (Svensson et al., J. Biol. Chem., 274, 9636-47, 1999). The protein has been associated with a number of tumors.

Surprisingly, this invention reveals overexpression of the fibromodulin protein in kidney cancer, thus indicating said protein as a novel human kidney tumor marker, more preferably as a human kidney tumor marker readily accessible from the bloodstream and, thus, useful as a target for ligand-based tumor targeting applications.

### Protein #7

The peroxidasin homolog [fragment] (also designated melanoma-associated antigen MG50) (Q92626) was originally identified by a cDNA subtraction approach, in which cDNA clones were isolated with a subtracted melanoma cDNA probe (melanoma cell line minus lung carcinoma cell line) after screening a melanoma expression library by in situ plaque hybridization (Hutchins et al., Cancer Res, 51, 1418-25, 1991).

Surprisingly, the present invention demonstrates that peroxidasin homolog [fragment] was only identified in tumor specimen but not in normal tissue of kidney and indicates a use of this protein as a tumor marker, more preferably as a kidney tumor marker, more preferably as a tumor marker readily accessible from the bloodstream.

It is noted that the peroxidasin homolog [fragment] has so far only been postulated from the demonstration of certain mRNA sequences but the existence of the protein has not yet been demonstrated in nature.

### Protein #8

The peptide MRAPGALLAR, surprisingly identified in kidney tumors only, matches the protein sequence of Probable G-protein coupled receptor 37 [precursor] (O15354).

The orphan G protein-coupled receptor GPR37 and related genes encode a subfamily of putative G protein-coupled receptors that are highly expressed in the mammalian central nervous system.

Toyota and coworkers found that GPR37 is one of the genes exhibiting hypermethylation of promoter-associated CpG-rich regions, termed CpG islands, in acute myeloid leukemia (AML) (Toyota et al., Blood, 97, 2823-9, 2001). Such hypermethylation can result in gene silencing that is clonally propagated through mitosis by the action of DNA-methyltransferase enzymes. Such methylation-associated silencing plays a pathological role in silencing tumor-suppressor genes in neoplasia.

Surprisingly, the present invention identifies GPR37 in tumor but not in normal kidney and demonstrates an overexpression of this protein in tumors, thus indicating this protein as a novel marker overexpressed in human tumors, more preferably as a human kidney tumor marker, most preferably as a human kidney tumor marker readily accessible from the bloodstream and, thus, useful as a target for ligand-based tumor targeting applications.

### Protein #9

The existence of the protein sidekick-1 [precursor] (Q8TEN9) has been postulated based on sequencing full-length cDNAs (Nagase, T et al., Kazusa DNA Research Institute, direct submission to the NCBI database), but was never experimentally proven.

Surprisingly, it was demonstrated that such a protein exists (by the identification of the peptide AELTDLK, which is specific for this protein), and that it is over-expressed in and/or around tumor neo-vasculature structures, thus opening vascular targeting biomedical applications.

### Protein #10

The protein Alpha1A-voltage-dependent calcium channel was shown to be mutated in a disease called spinocerebellar ataxia type 6 (SCA6), which is a autosomal dominant neurodegenerative disease (Toru, S et al., J. Biol. Chem. 275, 10893-8, 2000). Until now, no data concerning expression in normal tissue or tumor tissue is available.

Surprisingly, by the identification of the tryptic peptide RGALVGAPR the present invention demonstrates the over-expression in and/or around tumor neo-vasculature structures, thus indicating vascular targeting biomedical applications.

### Protein #11

The EMILIN2 protein [fragment] (elastin microfibril interfase located Protein 2, Q8N5L1) is an elastic fiber-associated glycoprotein. The mRNA expression of EMILIN2 protein has been shown by the group of Colombatti (Doliana, R et al., J Biol. Chem. 276, 12003-11, 2001). The protein has an expression pattern restricted to spinal cord, peripheral leukocytes, lung, placenta and fetal heart. In addition, the group presented an immuno-histochemistry of human leiomyosarcoma cells showing the partial co-localization of EMILIN1 and EMILIN2. The group of Forrest (Amma, L L et al., Mol Cell Neurosci. 23, 460-72, 2003) confirmed with a northern blot analysis the restricted expression pattern of the EMILIN2 protein to heart, lung and cochlea.

By the identification of the protein specific tryptic peptide RGALVGAPR the over-expression in and/or around tumor neo-vasculature structures was surprisingly demonstrated, thus, indicating vascular targeting biomedical applications.

### Protein #12

The Down syndrome critical region protein 8 (also designated malignant melanoma-associated protein 1) (Q96T75) has 6 different splice-isoforms. The tryptic peptide LFMPRPK is specific for 5 of these 6 splice-isoforms (see table 1).

Surprisingly, the over-expression of one or more of the 5 splice-isoforms Q96T75, Q6EXA9, Q684H4, Q96T75-2, and Q96T75-3 of the Down syndrome critical region protein 8 (of which in total 6 isoforms are published), in and/or around tumor neo-vasculature structures was demonstrated, thus indicating vascular targeting biomedical applications.

### Protein #13

The protein probable G-protein coupled receptor 113 [precursor] (Q81ZF5) was identified in the course of a large scale BLAST study focused on novel human G-protein coupled receptors (Fredriksson, R et al., FEBS Lett. 531, 407-14, 2002).

Surprisingly, this invention identifies the probable G-protein coupled receptor 113 [precursor] in tumors. By the identification of the protein specific tryptic peptide NKISYFR the over-expression in and/or around tumor neo-vasculature structures was demonstrated, thus indicating vascular targeting biomedical applications.

### Proteins #14

The protein database entries annexin A4 (P09525) and protein ANXA4 [fragment] (Q6LES2) have the same amino acid sequence except for the first 2 amino acids of Q6LES2. Thus, the difference in these two database entries may either be a consequence of a sequencing error or there are two isoforms of this protein existing. Zimmermann et al. (Zimmermann, U et al., Cancer Lett. 209, 111-8, 2004) showed in their paper concerning clear cell renal cell carcinoma that in normal cells annexin A4 is concentrated around the nucleus, whereas the protein is localized to the basolateral membrane in tumor cells. This suggests that the subcellular distribution of annexin IV correlates with the nature of the attachment of the cell to its neighborhood. These results indicate the possibility that annexin IV plays an important role in the morphological diversification and dissemination of the clear cell renal cell carcinoma.

Surprisingly, the present invention demonstrates the over-expression in and/or around tumor neo-vasculature structures by the identification of the protein specific tryptic peptide ISQTYQQQYGR, thus indicating vascular targeting biomedical applications.

### Protein # 15 (4×)

The uromodulin-like 1 [precursor] (also designated olfactorin) (Q5DIDO, Q5DIDO-2, Q5DIDO-3, and Q5DIDO-3) was identified as a novel membrane bound protein specifically expressed by olfactory and vomeronasal sensory neurons (Di Schiavi, E. et al., Eur. J. Neurosci. 21, 3291-300, 2005). The group transfected HEK-cells with olfactorin fused to a flag-tag and identified this fusion-protein with an anti-flag antibody.

Surprisingly, the present invention demonstrates the over-expression in and/or around tumor neo-vasculature structures by the identification of the protein-specific tryptic peptide IVNHNLTEKLLNR, thus indicating vascular targeting biomedical applications. In the prior art the protein olfactorin is also known as uromodulin-like protein, also having four different splice isoforms (see table 1).

### Protein #16

The protein scavenger receptor class F member 2 [precursor] (Q96GP6) has been demonstrated to be expressed in the mouse during embryogenesis in the hair follicle, skin and nasal epithelium as well as in the tongue and oral epithelia, rib bone undergoing ossification and in the medullar region of thymus (Hwang, M et al., Gene Expr Patterns. 5, 801-8, 2005).

Surprisingly, the over-expression in and/or around tumor neo-vasculature structures was demonstrated by the identification of the protein specific tryptic peptide GAGPARRR, thus indicating vascular targeting biomedical applications.

### Protein #17

The sushi domain-containing protein 2 [precursor] (Q9UGT4) was unambiguously identified by 2D-PAGE and MALDI mass spectrometry by the group of Lubec (Lubec, G. et al., J. Chem. Neuroanat. 26, 171-8, 2003) in the human cortical neuronal cell line HCN-2.

Surprisingly, the over-expression in and/or around tumor neo-vasculature structures was demonstrated by the identification of the protein specific tryptic peptide VAHQLHQR, thus indicating vascular targeting biomedical applications.

### Protein #18

The Tumor protein, translationally controlled 1 (TCTP) has been known for more than 20 years. In the review of Bommer and Thiele (Int. J. Biochem. Cell Biol. 36, 379-85, 2004) the importance of TCTP for cell growth and its anti-apoptotic activity are highlighted.

Surprisingly, the over-expression in and/or around tumor neo-vasculature structures was demonstrated by the identification of the protein specific tryptic peptide KWVKINNVK, thus indicating vascular targeting biomedical applications.

### Protein #19

The existence of the putative G-protein coupled receptor (Q8TDU0) was postulated based on sequence homology searches in the human genome (Takeda, S et al., FEBS Lett. 520, 97-101, 2002), but was never experimentally proven.

The present invention surprisingly demonstrates that such a protein actually exists (by the identification of the peptide LSVVEAPCR, which is specific for this protein), and also shows that it is over-expressed in and/or around tumor neo-vasculature structures, thus indicating vascular targeting biomedical applications.

### Protein #20

The existence of the hypothetical protein DKFZp686KO275 (Q7Z3A1) was postulated based on sequencing of full-length cDNAs (Wiemann, S et al., Molecular Genome Analysis, German Cancer Research Center (DKFZ), direct submission to the NCBI homepage), but was never experimentally proven.

The present invention surprisingly demonstrates that such a protein actually exists (by the identification of the peptide AGQGFGLR, which is specific for this protein), and also demonstrates that it is over-expressed in and/or around tumor neo-vasculature structures, thus indicating vascular targeting biomedical applications.

### Protein #21

The existence of the transmembrane protein TMEM55A (Q8N4L2) was postulated based on sequencing of full-length cDNAs (Strausberg, R L. et al., Proc. Natl. Acad. Sci. USA. 99, 16899-903, 2002), but was never experimentally proven.

The present invention surprisingly demonstrates that such a protein actually exists (by the identification of the peptide KISSVGSALPR, which is specific for this protein), and also demonstrates that it is over-expressed in and/or around tumor neo-vasculature structures, thus indicating vascular targeting biomedical applications.

### Protein #22

The existence of the hypothetical protein (Q8WYY4) was postulated based on sequencing of full-length cDNAs (Gu, J R. et al., National Laboratory For Oncogenes & Related Genes, Shanghai Cancer Institute, direct submission to the NCBI homepage), but was never experimentally proven.

The present invention surprisingly demonstrates that such a protein actually exists (by the identification of the peptide VLTAMVGK, which is specific for this protein), also demonstrates that it is over-expressed in and/or around tumor neo-vasculature structures, thus indicating vascular targeting biomedical applications.

### Protein #23

The existence of the family with sequence similarity 116, member A (Q8IWF6) was postulated based on sequencing of full-length cDNAs (Strausberg, R L. et al., Proc. Natl. Acad. Sci. USA. 99, 16899-903, 2002), but was never experimentally proven.

The present invention surprisingly demonstrates that such a protein actually exists (by the identification of the peptide GPAGLGPGSR, which is specific for this protein), and also demonstrates that it is over-expressed in and/or around tumor neo-vasculature structures, thus indicating vascular targeting biomedical applications.

### Protein #24

The protein UPF0260 protein C6orf66 (Q9P032), which is also known as HRPAP20 (hormone-regulated proliferation-associated protein 20 kDa), had been demonstrated to have an increased proliferation in the absence of hormone stimulation and augmented survival in the absence of serum in stable transfected MCF-7 (human breast carcinoma) cells. Karp et al. (Karp, C M et al., Cancer Res. 64, 1016-25, 2004) conclude that HRPAP20 is a phospho-protein that is required for proliferation and survival of hormone dependent tumor cells.

The present invention demonstrates for the first time the over-expression of the protein (by identification of the protein specific tryptic peptide MGALVIR) in a human tumor and more preferably in human tumor neo-vasculature structures, thus indicating vascular targeting biomedical applications.

### Protein #25

The existence of the protein cDNA FLJ45811 fis, clone NT2RP7014778 (Q6ZS59) was postulated based on sequencing of full-length cDNAs (Isogai, T et al., NEDO human cDNA sequencing project, direct submission to the NCBI homepage), but was never experimentally proven.

The present invention surprisingly demonstrates that such a protein actually exists (by the identification of the peptide QFWLGGVAR, which is specific for this protein), and also demonstrates that it is over-expressed in and/or around tumor neo-vasculature structures, thus indicating vascular targeting biomedical applications.

### Proteins #26 & 27

The identified peptide (EAFEAASR) does not allow for distinguishing between the two proteins hypothetical proteins DKFZp77901248 (Q6AHZ8) and beta-ureidopropionase (Q9UBR1). The RZPD homepage (http://www.rzpd.de) links the hypothetical protein DKFZp77901248 to beta-ureidopropionase. Beta-ureidopropionase is a protein whose deficiency leads to inborn errors of the pyrimidine degradation pathway (van Kuilenburg, A B et al., Hum Mol. Genet. 13, 2793-801, 2004). There are no data published implicating beta-ureidopropionase and cancer. Surprisingly, the present invention demonstrates the over-expression in and/or around tumor neo-vasculature structures, thus indicating vascular targeting biomedical applications.

### Protein #28

The existence of the hypothetical protein DKFZp434F1919 (Q9GZU6) and its isoform MDS011 (Q9GZT6) (the two proteins have 99.6% amino acid sequence identity) was postulated based on sequencing of full-length cDNAs (Ota, T et al., Nature Genetics 36, 40-45, 2004), but was never experimentally proven.

The present invention surprisingly demonstrates that such a protein actually exists (by the identification of the peptide IDAEIASLK, which is specific for this protein), and also demonstrates that it is over-expressed in and/or around tumor neo-vasculature structures, thus indicating vascular targeting biomedical applications.

### Protein #29

The expression of the protein cysteine-rich with EGF-like domain protein 2 [precursor] (six isoforms, see table 1) has been studied for different normal human tissues (northern blot) and brain (immuno-histochemistry) (Ortiz, J A et al., J. Neurochem. 95, 1585-96, 2005), but n6 data about tumor tissue is available.

Surprisingly, the present invention demonstrates the over-expression in and/or around tumor neo-vasculature structures, thus indicating vascular targeting biomedical applications.

### Protein #30

The existence of the UPF0378 family protein KIAA0100 [precursor] (Q5H9T4) was postulated based on sequencing cDNAs (Ottenwaelder, B. et al., The German cDNA Consortium, direct submission to the NCBI database), but was never experimentally proven.

The present invention surprisingly demonstrates that such a protein actually exists (by the identification of the peptide KLQAELK, which is specific for this protein), and also demonstrate that it is over-expressed in and/or around tumor neo-vasculature structures, thus indicating vascular targeting biomedical applications.

### Protein #31

In the present invention the peptide SPILAEVK was identified as part of the protein potassium voltage-gated channel subfamily H member 1 (O95259). Two isoforms of this protein exist, both of which contain this peptide (O95259 & O95259-2 (hEAG)).

Pardo and colleagues (Pardo et al., EMBO J., 18, 5540-5547, 1999) showed that the inhibition of this protein expression causes a significant reduction of cell proliferation. They showed expression of KCNH1 in breast and brain tumor cells. In addition, expression was detected by immunohistochemistry in cervix cancer (Farias et al., Cancer Res., 64, 6996-7001, 2004).

Surprisingly, this invention reveals overexpression of the protein potassium voltage-gated channel subfamily H member 1 in kidney cancer, thus, indicating said protein as a novel human kidney tumor marker, more preferably as a human kidney tumor marker readily accessible from the bloodstream and, thus, useful as a target for ligand-based tumor targeting applications.

### BRIEF DESCRIPTION OF THE FIGURES

FIG. 1. (A) is a schematic representation of the ex vivo kidney perfusion procedure employed for identifying tumor markers in the kidney's vasculature. Within two minutes after nephrectomy, tumor bearing kidney is perfused with a reactive ester derivative of biotin, thus washing away blood components and biotinylating accessible proteins. Biotinylated tissue specimens can be cut, processed separately for the purification of biotinylated proteins, yielding tryptic peptides which are separated by nano-HPLC and analyzed by matrix-assisted laser desorption ionization-tandem time-of-flight (MALDI-TOF/TOF) mass spectrometry. (B) Tumor-bearing kidney, cut in half, after ex vivo perfusion. Biotinylated structures in tissue sections are detected in the tumor portion (C) and in the normal kidney portion (D) using avidin-horseradish peroxidase-based staining protocols. Vascular structures are preferentially biotinylated in the neoplastic mass.

FIG. 2 shows the target validation by semi-quantitative PCR analysis of cDNA libraries [clear cell carcinoma, lane 1; granular cell carcinoma, lane 2; transitional cell carcinoma, lane 3; normal fetal kidney, lane 4; normal adult kidney, lane 5]. (*) Unlike other proteins, the confidence of the assignment of CEACAM3 with the MASCOT software was below 95% for the best peptide ion and is not unambiguous even after visual inspection of the MS-MS spectra.

FIG. 3 is the result of the immunohistochemical analysis of normal kidney and tumor sections with antibodies specific to periostin (A) and versican (B). Staining with anti-periostin antibody exhibited a low background staining in the normal kidney samples, but revealed a strong over-expression in 8 out of 8 tumors investigated. Versican was strongly over-expressed in 6 out of 8 tumors, but did not stain normal kidneys and other normal tissues. The staining reactions were absent in negative control experiments, omitting the primary antibodies. Scale bars=XXX.

FIG. 4 (A) shows the alignment of protein sequences of the various isoforms of periostin limited to the carboxy-terminal region of the protein where the alternative splicing occurs.

FIG. 4 (B) is a graphic representation of the different combinations of exons that correspond to the regions shown in Panel A. The isoforms identified in this study are named <<A >> to <<E >>. The pale blue rectangle in corresponds to the isoform-specific peptide EIPVTVYKPIIKK we identified (see FIG. 5).

FIG. 5 Identification of isoform-specific peptides of periostin. (A) MALDI-TOF spectrum of an HPLC fraction containing a peptide with a mass-to-charge ratio of 1527.98. (B) The sequence of this peptide (EIPVTVYKPIIKK) was determined by MALDI-TOF/TOF. In the table of theoretical (C) peptide fragment ions and (D) internal fragment ions, the identified ions are marked in bold. The peptide EIPVTVYKPIIKK (pale blue rectangle in Figure S1, panel B) encompasses the junction between two exons which are only present in isoforms Q15063-2 and <<B >>.

FIG. 6. (A) Polyacrylamide gel electrophoresis (SDS-PAGE) analysis of the purified recombinant fragment (47 kDa) of periostin that was used as antigen in the antibody phage display selections. (B) Graphic representation of the ELISA results with selected scFv clones after 2 rounds panning against FAS2-FAS4 or FAS4. Positive ELISA signals show binding affinity of the particular antibody clone to the antigen. The best clones were selected for further characterization. (C) Graphic representation of the results of the ELISA screening after the first round of affinity maturation of clone C2 against FAS2-FAS4.

FIG. 7. Immunohistochemical staining on tissue sections of a human kidney tumor. (A) Staining with the selected human monoclonal scFv antibody (clone C2) against periostin domains FAS2-FAS4 showed strong positive staining of extracellular structures mainly around tumor blood vessels (white arrow points to positive staining around a tumor blood vessel). (B) A negative control using the same staining protocol but omitting the scFv resulted in no specific staining.

FIG. 8. Immunohistochemical defection of periostin in several patients with renal clear cell carcinoma. Immunohistochemical staining revealed a strong over-expression of periostin in 8/8 tumors investigated. Black arrows point to selected areas with positive staining. Scale bars, 100 μm.

FIG. 9. Immunohistochemical detection of versican in several patients with renal clear cell carcinoma. Immunohistochemical staining revealed a strong over-expression of versican in 6/8 tumors investigated. Staining is located in the extracellular matrix, also around tumor blood vessels. Black arrows point to selected areas with positive staining. Scale bars, 25 μm.

FIG. 10. Polyacrylamide gel electrophoresis (SDS-PAGE) analysis of the purified recombinant fragment (26 kDa) of CEACAM3.

FIG. 11. Polyacrylamide gel electrophoresis (SDS-PAGE) analysis of the purified recombinant fragment (27 kDa) of fibromodulin.

FIG. 12. (A) Polyacrylamide gel electrophoresis (SDS-PAGE) analysis of the purified recombinant fragment (12 kDa) of melanoma-associated antigen MG50 that was used as antigen in the antibody phage display selections. (B) Graphic representation of the ELISA results with selected scFv clones after 3 rounds of panning against against the melanoma-associated antigen MG50 fragment. Positive ELISA signals show binding affinity of the particular antibody clone to the antigen. The best clones were selected for further characterization.

FIG. 13. Immunohistochemical staining on tissue sections of a human kidney tumor. (A) Staining with the selected human monoclonal scFv antibody (clone F6) against the melanoma-associated antigen MG50 domains fragment showed strong positive staining mainly around tumor blood vessels (black arrows point to some of the spots of positive staining around tumor blood vessels). (B) A negative control using the same staining protocol but omitting the scFv resulted in no specific staining.

FIG. 14. (A) Polyacrylamide gel electrophoresis (SDS-PAGE) analysis of the purified recombinant fragment (24 kDa) of Protein sidekick-1 that was used as antigen in the antibody phage display selections. (B) Graphic representation of the ELISA results with selected scFv clones after 2 rounds of panning against against the Protein sidekick-fragment. Positive ELISA signals show binding affinity of the particular antibody clone to the antigen. The best clones were selected for further characterization.

FIG. 15. (A) Polyacrylamide gel electrophoresis (SDS-PAGE) analysis of the purified recombinant fragment (37 kDa) of ANXA4 protein that was used as antigen in the antibody phage display selections. (B) Graphic representation of the ELISA results with selected scFv clones after 2 rounds panning against the recombinant ANXA4 protein. Positive ELISA signals show binding affinity of the particular antibody clone to the antigen. The best clones were selected for further characterization.

FIG. 16. Immunohistochemical staining on tissue sections of a human kidney tumor. (A) Staining with the selected human monoclonal scFv antibody (clone E11) against recombinant ANXA4 protein showed strong positive staining of tumor cells, including those around tumor blood vessels (white arrows point to some of the spots with positive staining; in fact, most of the tissue is positive). (B) A negative control using the same staining protocol but omitting the scFv resulted in only few areas with weak staining. Scale bars, 100 μm.

FIG. 17. Polyacrylamide gel electrophoresis (SDS-PAGE) analysis of the purified recombinant fragment (27 kDa) of UPF0378 family protein KIAA0100.

FIG. 18. (A) Schematic representation of the Potassium voltage-gated channel subfamily H member 1. The peptide used as antigen in the phage display selections corresponded to a the second extracellular loop of this membrane protein (see red arrow). (B) Graphic representation of the results of the ELISA screening after the first round of affinity maturation of clone H9 against the Potassium voltage-gated channel subfamily H member 1 peptide. Positive ELISA signals show binding affinity of the particular antibody clone to the antigen.

FIG. 19. Immunohistochemical staining on tissue sections of human tumors. Staining with the selected human monoclonal scFv antibody (clone H9) against Potassium voltage-gated channel subfamily H member 1 showed strong positive staining of tumor cell membranes in tissue section of human kidney tumors (A) and of human lung tumors (C) (black arrows point to positive staining of cell membranes). Negative controls using the same staining protocol but omitting the scFv resulted in no specific staining both in the kidney tumor tissue (B) and the lung tumor tissue (D).

FIG. 20. FACS experiment with HeLa cells. Treatment of the cells with scFv(H9) selected against Potassium voltage-gated channel subfamily H member 1 as primary antibody, mouse anti-c-myc (clone 9E10) as secondary and FITC-labeled anti-mouse Ig as tertiary antibody revealed a shift in the FACS (left panel) compared to cells treated with the same procedure only omitting the scFv (right panel), indicating a successful binding of scFv(H9) to the HeLa cells.

FIG. 21. Immunocytochemistry experiment with HeLa cells. Staining of cultured HeLa cells with the selected human monoclonal scFv antibody (clone H9) against Potassium voltage-gated channel subfamily H member 1 showed strong positive staining of tumor cell membranes (A) (the white arrow points to positive staining of cell membranes). Negative controls using the same staining protocol but omitting the scFv resulted in no specific staining (B).

The following examples are provided to better illustrate the present invention in more detail. They are not to be construed as limiting to the scope of the claims in any way.

In the specification and the appended claims, other than in the operating examples, or where otherwise indicated, all numbers expressing quantities of ingredients, reaction conditions, percentage identity, numbers of amino acids, numbers of nucleotides, etc. are to be understood as being modified in all instances by the term “about”. Furthermore, all numerical ranges cited herein are to be understood to specifically encompass all conceivable subranges thereof.

Example 1 below demonstrates the overexpression, of the marker proteins identified according to the invention in neovascular structures, in particular their overexpression in tumors, more specifically in kidney tumors.

Furthermore, examples 2 to 10 below demonstrate the recombinant production of selected vascular marker proteins (or fragments thereof) of the present invention and their utility as antigens for producing antibodies against these vascular marker proteins. Such antibodies (or other ligands with the same selective affinity) are useful for further characterizations and biomedical applications of these marker proteins. Furthermore, some of the examples prove the practical utility of selected marker proteins of the present invention for identifying neovascular structures, in particular neovascular structures in tumors.

### EXAMPLES

### Example 1

### Introduction

A chemical proteomic approach based on the ex vivo perfusion and biotinylation of accessible structures within surgically-resected human kidneys with tumor was used to gain information about accessible and abundant antigens which are over-expressed in human cancer. Biotinylated proteins were purified on streptavidin resin and identified using mass spectrometric methodologies, revealing 637 proteins, of which 184 were found in tumor specimens only and 223 in portions of normal kidneys only. Thirty of the accessible tumor-associated antigens identified with this methodology are suitable targets for antibody-based anti-cancer therapies.

The specific method used for the identification of accessible antigens in normal organs and in tumors is based on the terminal perfusion of tumor-bearing mice with reactive ester derivatives of biotin that was recently published by the present inventors (Rybak et al. 2005 supra). This methodology allows the efficient biotinylation of accessible proteins on the membrane of endothelial cells and on other structures (e.g. extracellular matrix components) which are readily accessible from the bloodstream. The purification of biotinylated proteins from organ lysates on streptavidin resin, followed by a comparative proteomic analysis based on mass spectrometry, allowed the identification of hundreds of accessible proteins, some of which were found to be differentially expressed in organs and in tumors.

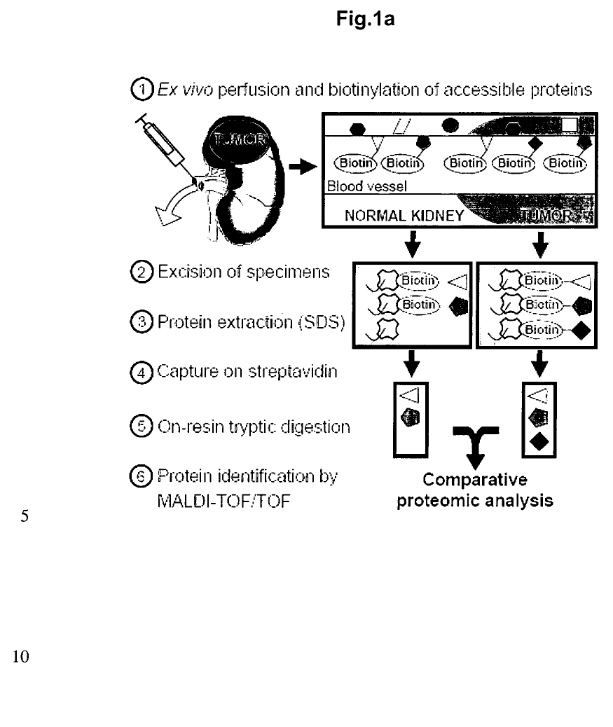

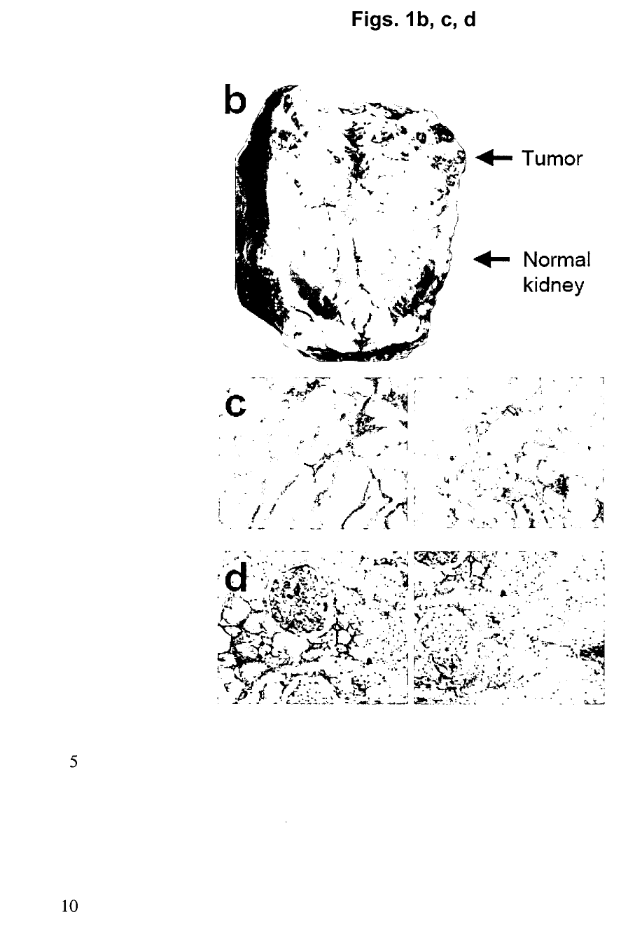

The above biotinylation procedure was applied to the ex vivo perfusion of three surgically-resected kidneys from patients with renal cell carcinoma (FIG. 1 and Table 2). This procedure lasted 7-9 minutes and allowed the efficient and selective labeling of vascular structures in the tumor portions (FIG. 1C), while both vascular and tubular structures were labeled in the normal kidney portions (FIG. 1D).

After biotinylation and quenching of excess biotinylation reagent with a primary amine-containing solution (Tris), specimens were excised, homogenized in the presence of SDS and loaded onto streptavidin resin, thus enriching for biotinylated proteins. A subsequent proteolytic digestion, followed by nanoHPLC peptide separation and mass spectrometric analysis by MALDI-TOF/TOF allowed for the identification of a total of 637 proteins in all specimens.

As expected, abundant proteins observed in both normal and neoplastic specimens included components of the extracellular matrix, such as collagens, laminin, perlecan, lumican, vitronectin, fibronectin, and tenascin. Proteins found exclusively in the normal kidney portions included the kidney-specific cadherin 16, several transporters, apolipoprotein E and uromodulin. A number of proteins were found exclusively in the tumor specimens. Some of these had previously been reported to be overexpressed in certain neoplastic structures [e.g., carbonic anhydrase IX, TEM4, Peroxidasin homolog [fragment], Down syndrome critical region protein 8, integrin alpha-1, ectonucleotide pyrophosphatase/-phosphodiesterase 3]. Only a small portion of the tumor antigens identified in this analysis (e.g., netrin receptor DCC, solute carrier family 2, facilitated glucose transporter member 1, neural cell adhesion molecule 1) have so far been reported in the “Human Protein Atlas”: a genome-wide initiative for the characterization of protein expression patterns in normal tissues and cancer.

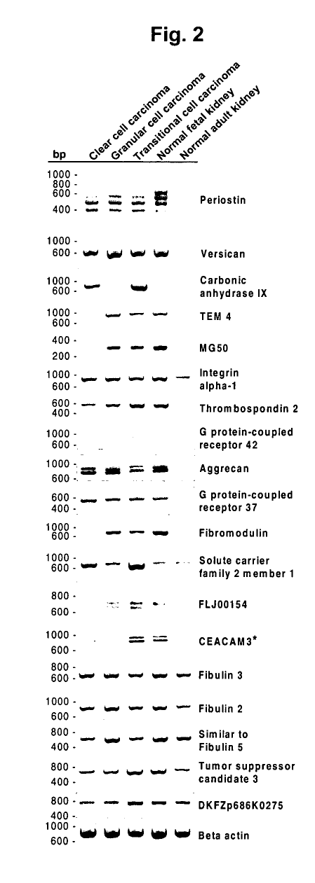

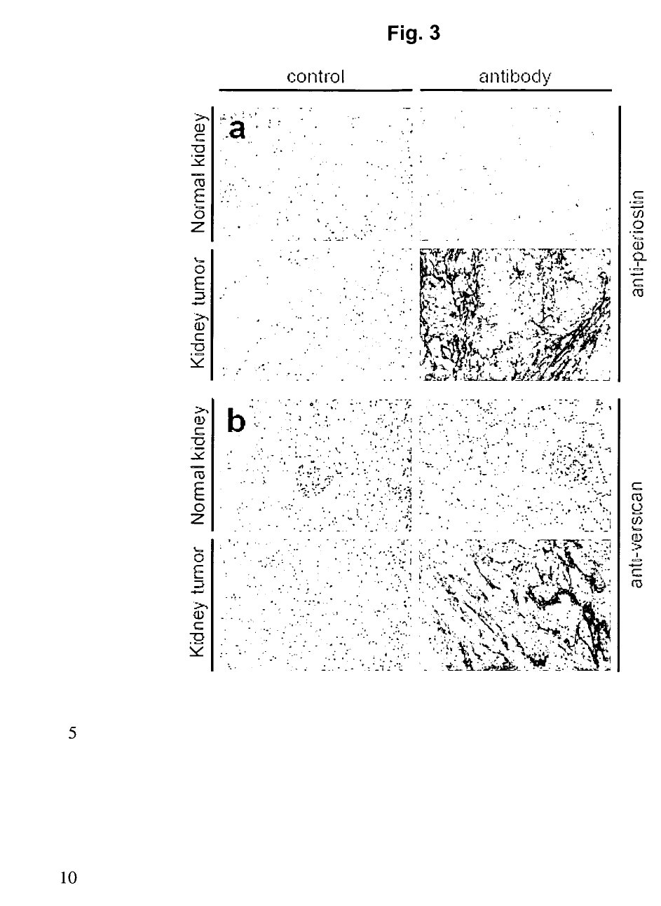

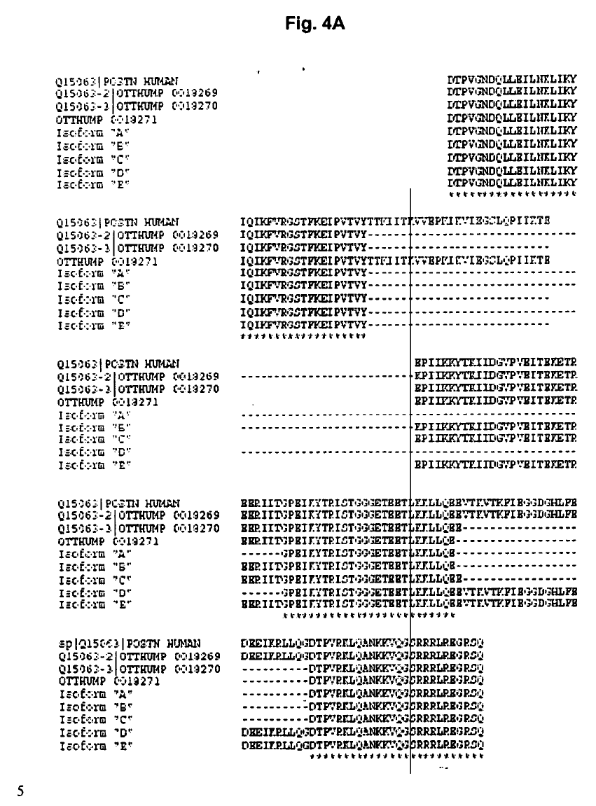

Since the detection of a protein in the tumor specimens might in principle reflect not only a preferential pattern of expression, but also a differential accessibility to the biotinylation reagent, selected protein candidates were characterized both by immunohistochemistry and by PCR analysis of cDNA libraries. Periostin was the most abundant tumor-associated antigen in this analysis and represents a particularly interesting marker, having been found to be up-regulated in epithelial ovarian tumors, breast cancer, at the periphery of lung carcinomas, and in colorectal cancers and their liver metastases. The existence of five different splice isoforms of periostin in human has been reported, but sequences of only three isoforms are published (Swiss-Prot/TrEMBL and NCBI Protein). However, six isoform sequences were identified in the PCR analysis of cDNA libraries, with different relative abundance in normal, fetal and tumor kidney (FIG. 2 and FIG. 4). An immunohistochemical analysis of normal kidney and clear cell carcinoma specimens revealed a striking over-expression of periostin in the tumors, with a prominent vascular and stromal pattern of staining (FIG. 3). Similarly, versican was found to be more abundant in fetal and tumor specimens, both by PCR (FIG. 2) and by immunohistochemical analysis (FIG. 3).

Some of the putative tumor-associated antigens were found to be present also in cDNA libraries of normal kidney (FIG. 2), including fibulins, tumor suppressor candidate 3 (N33 protein) and the hypothetical protein DKFZp686K0275 [fragment] (FIG. 2). By contrast, a number of interesting markers yielded substantially stronger PCR bands in fetal and tumor specimens including carbonic anhydrase IX, TEM4, Peroxidasin homolog [fragment], integrin alpha-1, thrombospondin 2, putative G-protein-coupled receptor 42, aggrecan, probable G-protein-coupled receptor 37 [precursor], fibromodulin, solute carrier family 2, facilitated glucose transporter member 1, and Protein sidekick-1 [precursor] (FIG. 2).

Among the 637 proteins identified in this analysis, approx. 20% corresponded to intracellular proteins. While some more abundant intracellular proteins (e.g. actin, tubulin, keratin, histones) could be recovered either by stickiness to the streptavidin resin or as a consequence of biotinylating necrotic structures ex vivo, some intracellular proteins have been reported to become accessible on the surface of proliferating endothelial cells.

### Materials and Methods

### Patients

This study was started upon approval by the ethical committee of the University Hospital of Liege (Belgium). Criteria adopted for patient selection were as follows: 1) diagnosis of a tumor highly compatible with a clear cell carcinoma of the kidney, as assessed by routine ultrasound and abdomen CT scan; 2) a therapeutic indication for a total nephrectomy; 3) a tumor size and localization that allowed to clearly distinguish healthy portions of the kidney to be used as normal controls. Immunohistochemical procedures compatible with the detection of specific proteins without biotin interference were adopted for the diagnostic histopathological analysis. Patient's informed consent was obtained and serology for negativity to HIV and Hepatitis A, B, and C was performed. For specific information about the patients see Table S1.

### Ex Vivo Vascular Perfusion

Surgery was performed according to a standard procedure, which includes the ligation and section of renal artery, vein, and ureter, and subsequent nephrectomy. The renal artery carried a longer suture for immediate identification in the perfusion step. Within 2 min after nephrectomy, the renal artery was cannulated, the renal vein was opened (by removing the suture) to allow outflow of the perfusate, and perfusion via the renal artery was started. Kidneys were first perfused 7-9 min with 500 ml of a 1 mg/ml solution of sulfo-NHS-LC-biotin in PBS, washing away blood components and labelling accessible primary amine-containing structures with biotin. Immediately afterwards, a second perfusion step with 450 ml PBS containing a 50 mM solution of the primary amine Tris-(hydroxymethyl)-aminomethane (Tris) was performed for 8-9 min to quench unreacted biotinylation reagent. All perfusion solutions contained 10% dextran-40 as a plasma expander and were pre-warmed to 40° C. Both perfusion steps were performed with a pressure of 100-150 mm Hg. Successful perfusion was indicated by the wash out of blood during the first minutes of perfusion and subsequent flow of clear perfusate out of the renal vein. After perfusion, the organs were washed with 50 mM Tris in PBS, dried, rubbed with black ink to allow the later pathologic investigation of surgical margins, and cut in half along the sagittal axis (starting at the external medial edge) with a blade. Successful perfusion resulted in a whitish color of the tissue. Specimens from the tumor and from the normal kidney tissue (unaffected by the tumor) were excised (from the well perfused, whitish parts) and immediately snap-frozen for proteomic and histochemical analyses, or paraformaldehyde-fixed and paraffin-embedded for histochemical analyses. As a negative control, unperfused organs after nephrectomy were cut in half and specimens were taken as described above from the tumor and from normal kidney tissue. For specific information about the examined organs see Table 2.

Histochemical Staining of Tissue Sections with Avidin-Biotinylated Peroxidase Complex

Sections from paraformaldehyde-fixed, paraffin-embbeded tissue specimens were stained with avidin-biotin-peroxidase complex (Vectastain Elite ABC kit (Vector Laboratories, Burlingame, Calif., USA)) according to standard procedures.

### Preparation of Protein Extracts for Proteomic Analysis

Specimens from healthy kidney and clear cell carcinoma tissue of human cancer patients were resuspended in 40 μl per mg tissue of a lysis buffer containing 2% SDS, 50 mM Tris, 10 mM EDTA, CompleteE proteinase inhibitor cocktail (Roche Diagnostics, Mannheim, Germany) in PBS, pH 7.4 and homogenized using an Ultra-Turrax T8 disperser (IKA-Werke, Staufen, Germany) applying six intervals of 2 min full power and 2 min standby at moderate cooling. Homogenates were sonicated (6 intervals of 30 s and 1 min standby at moderate cooling) using a Vibra-cell (Sonics, New Town, Conn., USA), followed by 15 min incubation at 99° C. and 20 min centrifugation at 15000×g. The supernatant was used as total protein extract. Protein concentration was determined using the BCA Protein Assay Reagent Kit (Pierce).

### Purification of Biotinylated Proteins

SA-sepharose slurry (64 μl/mg total protein) was washed three times in buffer A (NP40 1%, SDS 0.1% in PBS), pelleted and the supernatant removed. Fifteen milligrams of total protein extract from the different specimens were mixed to the pellet of SA-Sepharose. Capture of biotinylated proteins was allowed to proceed for 2 h at RT in a revolving mixer. The supernatant was removed and the resin washed three times with buffer A, two times with buffer B (NP40 0.1%, NaCl 1 M in PBS), and once with 50 mM ammonium bicarbonate. Finally, the resin was resuspended in 400 μl of a 50 mM solution of ammonium bicarbonate and 20 μl of sequencing grade modified porcine trypsin (stock solution of 40 ng/μl in 50 mM ammonium bicarbonate) (Promega, Madison, Wis., USA) were added. Protease digestion was carried out overnight at 37° C. under constant agitation. The supernatants were collected and trifluoracetic acid was added to a final concentration of 0.1%. Peptides were desalted, purified and concentrated with C18 microcolumns (ZipTip C18, Millipore, Billerica, Mass., USA). After lyophilization peptides were stored at −20° C.

Nano Capillary-HPLC with Automated Online Fraction Spotting onto MALDI Target Plates

Tryptic peptides were separated by reverse phase high performance liquid chromatography (RP-HPLC) using an UltiMate nanoscale LC system and a FAMOS microautosampler (LC Packings, Amsterdam, The Netherlands) controlled by the Chromeleon software (Dionex, Sunnyvale, Calif., USA). Mobile phase A consisted of 2% acetonitrile and 0.1% trifluoroacetic acid (TFA) in water, mobile phase B was 80% acetonitrile and 0.1% TFA in water. The flow rate was 300 nl/min leading to a pressure of mobile phase A of ˜170 bar. Lyophilized peptides derived from the digestion of biotinylated proteins affinity purified from 1.5 mg of total protein were dissolved in 5 μl of buffer A and loaded on the column (inner diameter: 75 μm, length 15 cm, filled with C18 PepMap 100, 3 μm, 100 Å beads; LC Packings). The peptides were eluted with a gradient of 0-30% B for 7 min, 30-80% B for 67 min, 80-100% B for 3 min and 100% B for 5 min; the column was then equilibrated with 100% A for 20 min before analyzing the next sample. Eluting fractions were mixed with a solution of 3 mg/ml α-cyano-4-hydroxy cinnamic acid, 277 pmol/ml neurotensin (internal standard), 0.1% TFA, and 70% acetonitrile in water and deposed on a 192-well MALDI target plate using an on-line Probot system (Dionex). The flow of the MALDI-matrix solution was set to 1.083 μl/min. Thus, each fraction collected during 20 s contained 361 nl MALDI-matrix solution and 100 nl sample. The end-concentration of neurotensin was 100 fmol per sample well.

### MALDI-TOF/TOF Mass Spectrometry

Matrix-assisted laser desorption ionization tandem time-of-flight (MALDI-TOF/TOF) mass spectrometric analysis was carried out with the 4700 Proteomics Analyzer (Applied Biosystems, Framingham, Mass.). All spectra were acquired with an Nd:YAG laser working at a laser frequency of 200 Hz. For precursor ion selection, all fractions were measured in MS mode before MS/MS was performed. A maximum of 15 precursors per sample spot were selected for subsequent fragmentation by collision induced dissociation. Criteria for precursor selection were a minimum S/N of 60 and shot-to-shot precursor mass tolerance of 120 ppm. Spectra were processed and analyzed by the Global Protein Server Workstation (Applied Biosystems), which uses internal MASCOT (Matrix Science, UK) software for matching MS and MS/MS data against databases of in silico digested proteins. The data obtained were screened against a human database downloaded from the NCBI homepage (http://www.ncbi.nim.nih.gov/). The number of missed cleavages was set to 2. Protein identifications, performed by means of the MASCOT software, were considered to be correct calls within the 95% confidence interval for the best peptide ion. Selected hits within the confidence interval between 90% and 95% were verified by manual inspection of the spectra.

### Immunohistochemical Staining

Sections from paraformaldehyde-fixed, paraffin-embbeded tissue specimens were stained by the immunoperoxidase technique (Vectastain Elite ABC kit (Vector Laboratories, Burlingame, Calif., USA)) according to standard procedures. The immunoaffinity-purified rabbit polyclonal anti-periostin antibody (Biovendor, Heidelberg, Germany) and the monoclonal anti-versican antibody (clone 12C5; Developmental Studies Hybridoma Bank, University of Iowa, Ames, Iowa, USA) were used in a dilution of 1:500.

### PCR Analysis

A human kidney tumor cDNA panel containing cDNAs from clear cell carcinoma, granular cell carcinoma, transitional cell carcinoma, normal adult and fetal kidney were purchased from BioChain (Hayward, Calif., USA). Polymerase chain reaction (PCR) was performed using the Hot Start Taq Polymerase kit (Qiagen, Hilden, Germany). PCR conditions were as follows: denaturation at 95° C. for 15 min, followed by 35 cycles of denaturation at 94° C. for 1 minute, annealing at 54° C. for 1 minute and elongation at 72° C. for 1 minute. A final step of elongation at 72° C. for 10 min was performed. Primer sequences are available upon request. The products of the PCR reaction were analyzed by 2% agarose gel electrophoresis, stained by ethidium bromide, and imaged using the BioDoc-It imaging system (UVP, Upland, Calif., USA). For the analysis of periostin splice isoforms, bands were cut out from the agarose gel and sequenced (Big Dye Terminator v1.1 Cycle Sequencing kit; ABI PRISM 310 Genetic Analyzer; Applied Biosystems, Foster City, Calif., USA).

### Identification of New Splice Variants of Periostin

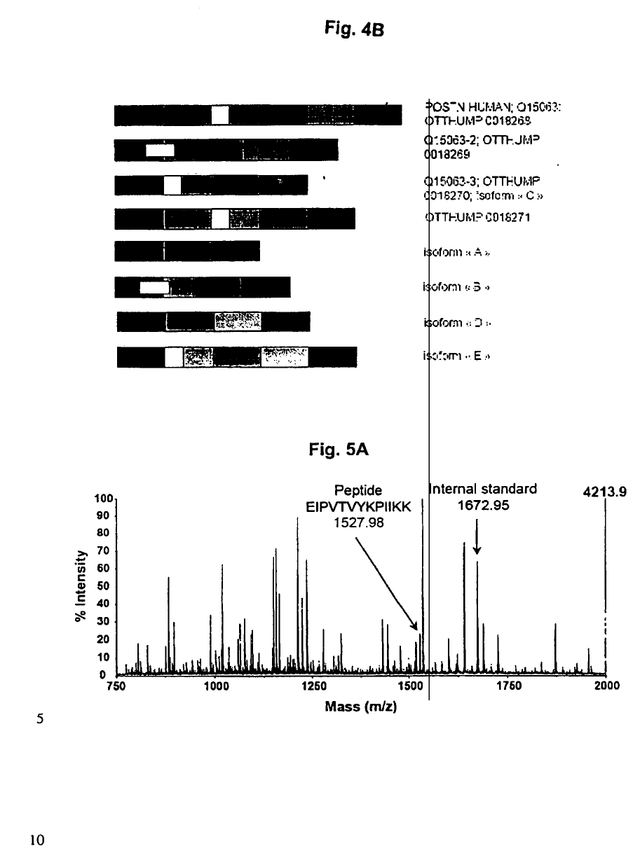

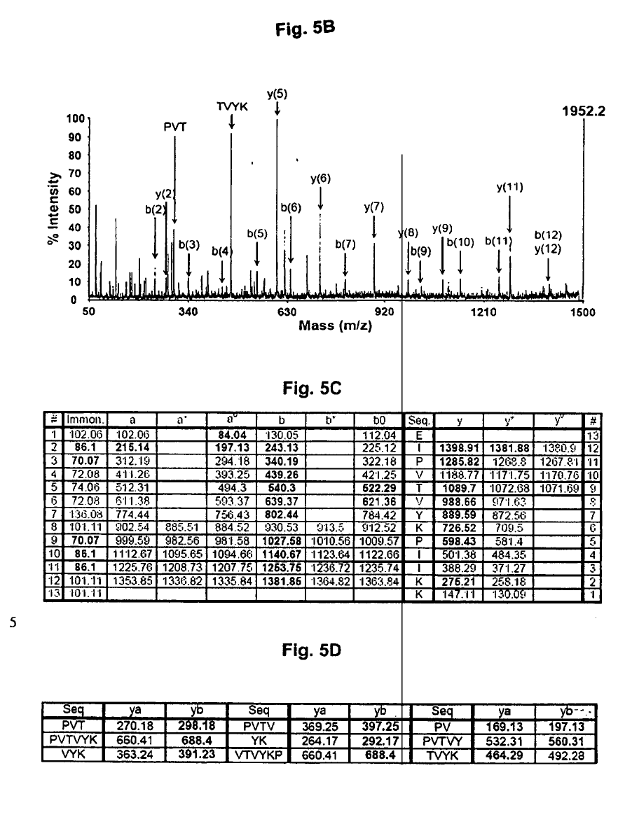

For human periostin, Takeshita and colleagues have reported that five alternative spliced transcripts can be produced, and that all splicing events of periostin occur within the C-terminal region. The possibility that some particular isoform might be selectively expressed in tumors and not expressed in normal tissue (in analogy to the expression pattern of the well-characterized ED-B containing isoform of fibronectin and the C domain-containing large tenascin C isoform) lead to the design of primers for the amplification of the alternative spliced domains by PCR on human cDNA libraries. The PCR amplification of the C-terminal region of periostin (FIG. 5) revealed at least five different splice variants. This would be consistent with the findings of Takeshita and coworkers (supra), who, however, have not published the sequences of the isoforms which they have found. An analysis of the existing databases reveals the existence of three isoforms (SwissProt numbers Q15063, Q15063-2 and Q15063-3) and of an EST clone (OTTHUMP 0018271), corresponding to a fourth different periostin isoform. However, excision of the amplified bands shown in FIG. 2 and their sequencing yielded six different isoforms, four of which did not correspond to any of the known ones. The full length periostin and an isoform corresponding to isoform Q15063-3 were also identified by sequencing in the analysis.

### Identification of Isoform-Specific Peptides of Periostin

It is important to note that the above proteomic analysis was able to identify, peptides which are isoform specific that are specifically present in tumor samples only, meaning that they encompass junctions of exons (or exon portions) which only exist in a particular isoform and not in others. (see FIG. 5 (A) MALDI-TOF spectrum of an HPLC fraction containing a peptide of 1527.5797 m/z. (B and C)) The sequence (EIPVTVYKPIIKK) was determined by collision-induced dissociation in a MALDI-TOF/TOF experiment. The peptide EIPVTVYKPIIKK (pale blue rectangle in FIG. 4, panel B) encompasses the junction between two exons which are only present in isoforms Q15063-2 and <<B >>. Another example of an isoform-specific peptide is represented by the peptide of sequence AELTDLK that encompasses a domain junction only present in the hypothetical protein FLJ00154, a protein specifically detected only in the tumor portion of human kidneys, and which represents a minor transcript of the human homologue of Sidekick-like protein 1 (data not shown). The identification of tumor-specific isoforms of proteins otherwise present in other normal tissues deserves allows for producing isoform-specific antibodies that are selective in that they are able to discriminate among the various isoforms of the same protein, and these antibodies will provide potential targets for tumor therapy.

### Results

TABLE 2
Specification of cases involved in this study. The kidneys of five patients
of different age and sex affected by tumors of the specified size and stage
were analyzed. Three organs were biotinylated ex vivo as described above
whereas the other two unperfused kidneys specimens were collected and
analyzed as negative controls.

| | | | Tumor | | |
|---|---|---|---|---|---|
| | | | size | | |
| # | Age | Sex | (cm) | Stage | Experiment |
| 1 | 68 | male | 10 | pT2 | ex vivo biotinylation |
| 2 | 52 | female | 3 | pT1A | ex vivo biotinylation |
| 3 | 63 | | 12 | pT3A | ex vivo biotinylation |
| 4 | 46 | | 15.5 | pT3B | unperfused negative |
| | | | | | control |
| 5 | 66 | | 3 | pt1 | unperfused negative |
| | | | | | control |

TABLE 3
Selection of putative membrane proteins and extracellular matrix
proteins identified in normal kidney, in the tumor portion, or both.
Numbers indicate in how many of the three patients, whose kidneys
were biotinylated ex vivo, the protein was identified.

| | | # Patients | |
|---|---|---|---|
| | | (total = 3) | |
| | | normal | |
| # | Swiss Prot. Acc. No. | kidney | tumor |
| 1 | Q15063/Q5VSY8/Q5VSY7/Q5VSY6 | 0 | 3 |
| 2 | O15529/O14843 | 0 | 2 |
| 3 | P11166 | 0 | 2 |
| 4 | P13611 | 0 | 2 |
| 5 | Q6UY47/Q3KPI0/O75296 | 0 | 1 |
| 6 | Q8IV47 | 0 | 1 |
| 7 | Q92626 | 0 | 1 |
| 8 | O15354 | 0 | 1 |
| 9 | Q8TEN9 | 0 | 1 |
| 10 | Q9NS88 | 0 | 2 |
| 11 | Q8N5L1 | 0 | 2 |
| 12 | Q96T75/Q6EXA9/Q684H4/Q96T75- | 0 | 1 |
| | 2/Q96T75-3 | | |
| 13 | Q8IZF5 | 0 | 1 |
| 14 | Q6LES2/P09525 | 0 | 1 |
| 15 | Q5DID0/Q5DID0-2/Q5DID0-3/ | 0 | 1 |
| | Q5DID0-4 | | |
| 16 | Q96GP6 | 0 | 1 |
| 17 | Q9UGT4 | 0 | 1 |
| 18 | Q5W0H4 | 0 | 2 |
| 19 | Q8TDU0 | 0 | 2 |
| 20 | Q7Z3A1 | 0 | 1 |
| 21 | Q8N4L2 | 0 | 1 |
| 22 | Q8WYY4 | 0 | 1 |
| 23 | Q8IWF6 | 0 | 2 |
| 24 | Q9P032 | 0 | 1 |
| 25 | Q6ZS59 | 0 | 1 |
| 26 | Q6AHZ8 and/or Q9UBR1 | 0 | 1 |
| and/or | | | |
| 27 | | | |
| 28 | Q9GZU6/Q9GZT6 | 0 | 1 |
| 29 | Q6UXH1/Q6UXH1-2/Q6UXH1-3/ | 0 | 1 |
| | Q6UXH1-4/Q6UXH1-5/Q6UXH1-6 | | |
| 30 | Q5H9T4 | 0 | 1 |
| 31 | O95259/O95259-2 | 0 | 1 |

### Example 2

### Introduction

For assessing periostin's utility as tumor marker recombinant fragments of this protein were cloned and expressed. Human monoclonal antibodies against the recombinant fragments were produced and tested in ELISA and immuno-histochemistry experiments.

### Materials and Methods

Recombinant protein fragments corresponding to the amino acid sequence positions 232-632 (FAS2-FAS4) and 496-632 (FAS4) of periostin (SEQ ID NO: 1) were cloned for use as antigen for biopanning experiments. The fragments were expressed in E. coli strain TG1 using pQE12 vector (Qiagen, Hilden, Germany). Proteins were purified from E. coli lysates using Ni-NTA columns (Qiagen). Antibodies in single chain Fv format (scFv) against the periostin fragments were selected from the ETH-2-Gold phage display library according to the procedure reported in Silacci et al., Proteomics. 2005 June; 5(9):2340-50. ELISA screening for clones expressing scFv antibody binding the antigen and immunohistochemical staining using single chain Fv preparations on sections of freshly frozen tissue samples were performed as described previously in Silacci et al., Proteomics. 2005 June; 5(9):2340-50 and Brack et al., Clin. Cancer Res. 2006 May 15; 12(10):3200-8. Affinity maturation of selected antibodies was done as described in Brack et al., Clin. Cancer Res. 2006 May 15; 12(10):3200-8. Immunohistochemical stainings with the commercial immunoaffinity-purified rabbit polyclonal anti-periostin antibody (Biovendor, Heidelberg, Germany) were performed as described in Example 1.

### Results

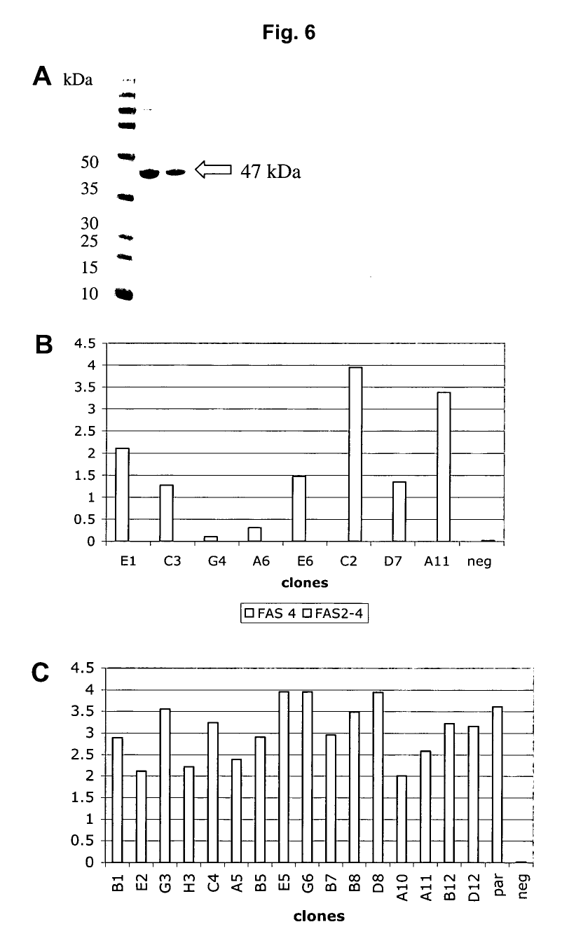

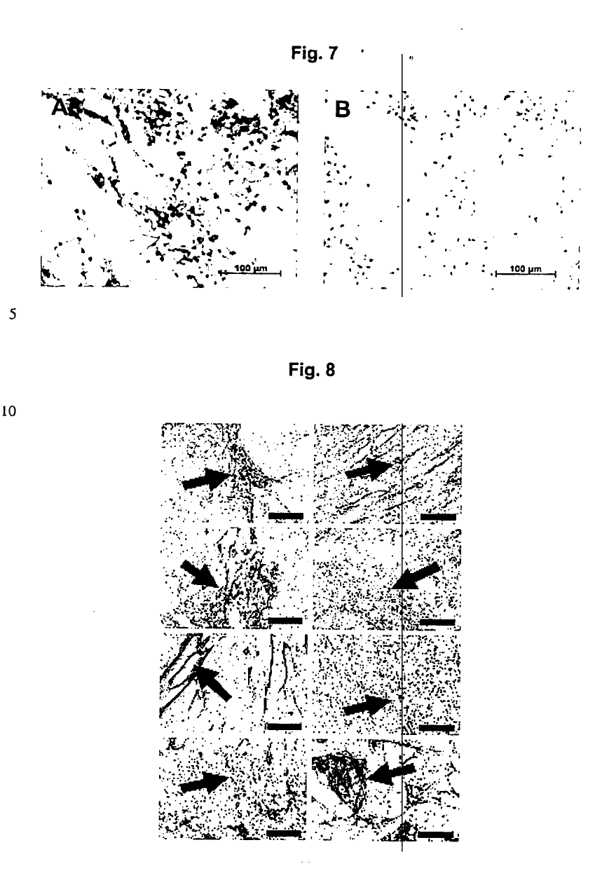

The periostin domains FAS2-FAS4 or the domain FAS4 were cloned and expressed. FIG. 6A shows an SDS-PAGE analysis of the purified recombinant protein FAS2-FAS4 (band at 47 kDa). Phage display selections against this protein resulted in numerous clones expressing scFv antibodies specific to FAS2-4 and to FAS4 as shown by ELISA on the coated recombinant protein (see FIG. 6B). The best clone C2 against FAS24 was further affinity matured to obtain an antibody with higher affinity (see ELISA results in FIG. 6C). The selected anti-periostin antibody C2 was used in an immunohistochemical analysis, which showed positive staining of vascular and extracellular matrix structures on tissue sections of human kidney tumors (see FIG. 7A). The negative control, for which sections from the same tissue were submitted to the same staining protocol only omitting the scFv, showed no positive staining (see FIG. 7B). Furthermore, a commercial antibody against periostin (see also Example 1) was used to evaluate the periostin expression in kidney tumors of different patients (see FIG. 8). Eight out of eight patients tested showed positive anti-periostin staining in the kidney tumor. These results demonstrate that human monoclonal antibodies against periostin can be produced and used for the detection of this antigen in neovascular structures of human cancer tissues. The immunohistochemical results indicate that periostin is expressed at high levels in human kidney tumors of most if not all patients.

### Example 3

### Introduction

To further study the versican antigen, we have performed an immunohistochemical analysis on kidney tumor sections of different patients.

### Materials and Methods

Immunohistochemical stainings with the monoclonal anti-versican antibody (clone 12C5) on paraformaldehyde-fixed, paraffin-embedded sections of human kidney tumors were performed as described in Example 1.

### Results

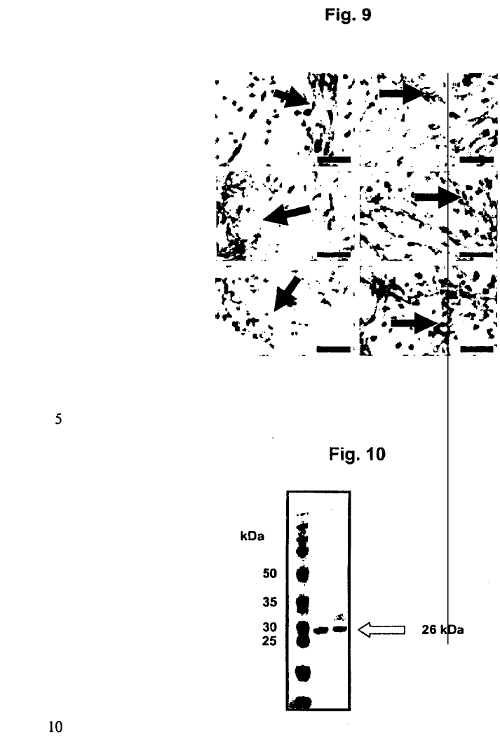

Furthermore, a commercial antibody against versican (see also Example 1) was used to evaluate the periostin expression in kidney tumors of different patients (see FIG. 9). Six out of eight patients tested showed positive anti-versican staining in the kidney tumor, either more diffuse in the extracellular matrix or more restricted to areas around tumor blood vessels. These results indicate that versican is expressed at high levels in human kidney tumors of most patients and is likely to be accessible from the vasculature.

### Example 4

### Introduction

To assess the CEACAM3 antigen a recombinant fragment of this protein was cloned and expressed for use as an antigen for the selection of antibodies by phage display.

### Materials and Methods

A recombinant protein fragment corresponding to the amino acid sequence positions 36-236 of CEACAM3 (SEQ ID NO: 25) was cloned and expressed as described in example 2 for use as antigen for biopanning experiments.

### Results

The CEACAM3 domains corresponding to the amino acid sequence positions 36-236 was cloned and expressed. FIG. 10 shows an SDS-PAGE analysis of the purified recombinant protein (band at 26 kDa). These results indicates that CEACAM3 fragments can be prepared in order to use them as antigen in antibody selection. Such antibodies could be used for a further validation of CEACAM3 as a vascular tumor marker similar to example 2.

### Example 5

### Introduction

To assess the utility of fibromodulin as antigen a recombinant fragment of this protein was cloned and expressed for the selection of antibodies by phage display.

### Materials and Methods

A recombinant protein fragment corresponding to the amino acid sequence positions 94-315 of CEACAM3 (SEQ ID NO: 25) was cloned and expressed as described in example 2 for use as antigen for biopanning experiments.

### Results

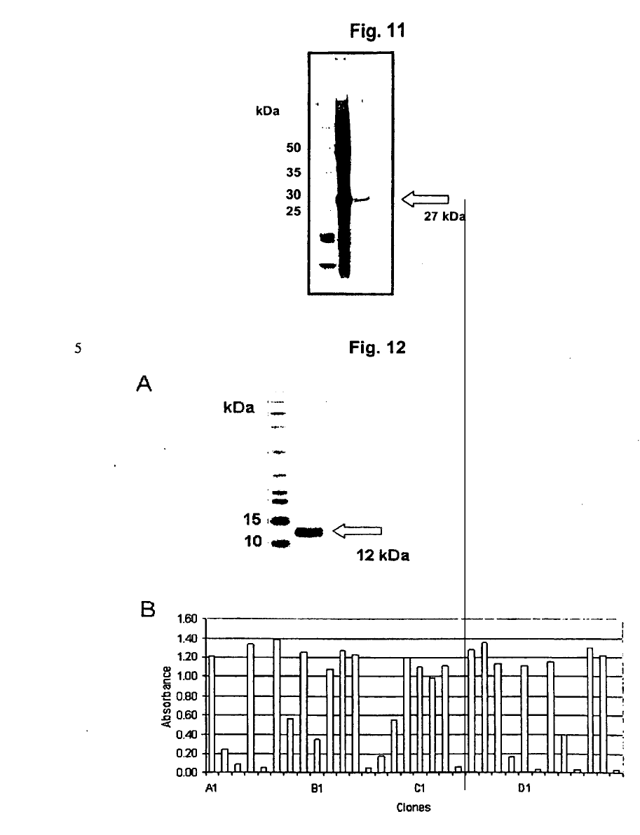

The fibromodulin fragment corresponding to the amino acid sequence positions 94-315 was cloned and expressed. FIG. 11 shows an SDS-PAGE analysis of the purified recombinant protein (band at 27 kDa). This result indicates that fibromodulin fragments can be prepared in order to use them as antigen in antibody selections. Such antibodies could be used for a further validation of fibromodulin as a vascular tumor marker similar to example 2.

### Example 6

### Introduction

For assessing peroxidasin homolog [fragment] for use as tumor marker, a recombinant fragment of this protein was cloned and expressed. Human monoclonal antibodies against the recombinant fragment were produced and tested in ELISA and immunohistochemistry experiments.

### Materials and Methods

A recombinant protein fragment corresponding to the amino acid sequence positions 539-632 of peroxidasin homolog [fragment] (SEQ ID NO: 33) was cloned and expressed, and antibody phage display selections, ELISA screening and immunohistochemistry performed as described in example 2.

### Results

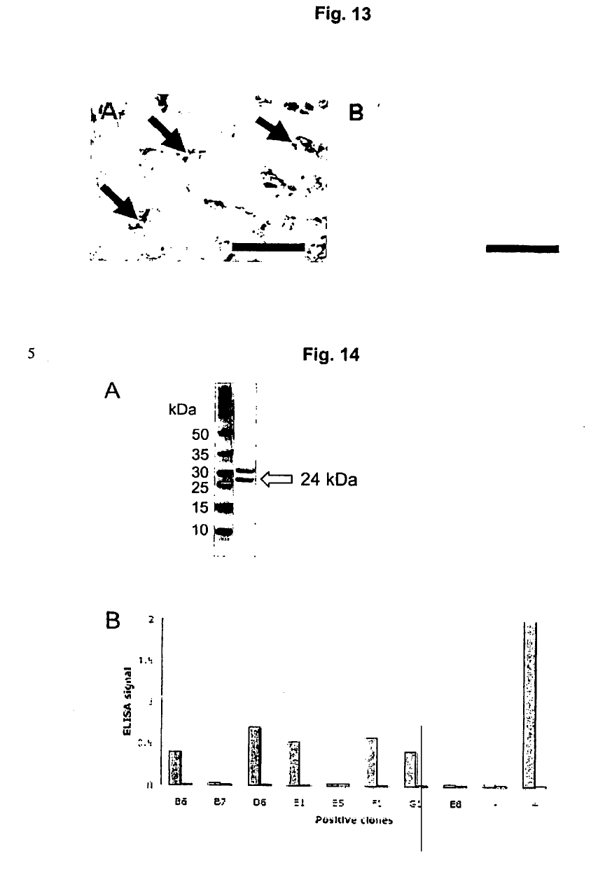

The peroxidasin homolog fragment was cloned and expressed. FIG. 12A shows an SDS-PAGE analysis of the purified recombinant protein fragment (band at 12 kDa). Phage display selections against this protein resulted in numerous clones expressing scFv antibodies specific to the recombinant peroxidasin homolog fragment as shown by ELISA on the coated recombinant protein (see FIG. 12B). A selected anti-peroxidasin homolog [fragment] antibody was used in an immunohistochemical analysis, which showed positive staining mainly around tumor blood vessels on tissue sections of human kidney tumors (see FIG. 13A). The negative control, omitting the scFv, showed no positive staining (see FIG. 13B). These results demonstrate that human monoclonal antibodies against peroxidasin homolog [fragment] can be produced and used for the detection of this antigen in neovascular structures of human cancer tissues. The immunohistochemical results indicate that peroxidasin homolog [fragment] is expressed at high levels in human tumor neovasculature.

### Example 7

### Introduction

For assessing the potential of Protein sidekick-1 as tumor marker a recombinant fragment of this protein was cloned and expressed. Human monoclonal antibodies against the recombinant fragment were produced and tested in ELISA.

### Materials and Methods

A recombinant protein fragment corresponding to the amino acid sequence positions 851-1052 of Protein sidekick-1 (SEQ ID NO: 37) was cloned and expressed, and antibody phage display selections and ELISA screening performed as described in example 2.

### Results

The Protein sidekick-1 fragment was cloned and expressed. FIG. 14A shows an SDS-PAGE analysis of the purified recombinant protein fragment (band at 24 kDa). Phage display selections against this protein resulted in several clones expressing scFv antibodies specific to the recombinant Protein sidekick-1 fragment as shown by ELISA on the coated recombinant protein (see FIG. 14B). These results demonstrate that human monoclonal antibodies against Protein sidekick-1 can be produced against recombinant antigen fragments. These antibodies could be used for a further validation of Protein sidekick-1 as a tumor marker, similar to the process described in example 2.

### Example 8

### Introduction

For assessing the ANXA4 protein for use as tumor marker a recombinant fragment of this protein was cloned and expressed. Human monoclonal antibodies against the recombinant fragments were produced and tested in ELISA and immuno-histochemistry experiments.

### Materials and Methods

A recombinant protein fragment corresponding to the amino acid sequence positions 3-321 of ANXA4 protein (SEQ ID NO: 52) was cloned and expressed, and antibody phage display selections, ELISA screening and immunohistochemistry performed as described in example 2.

### Results

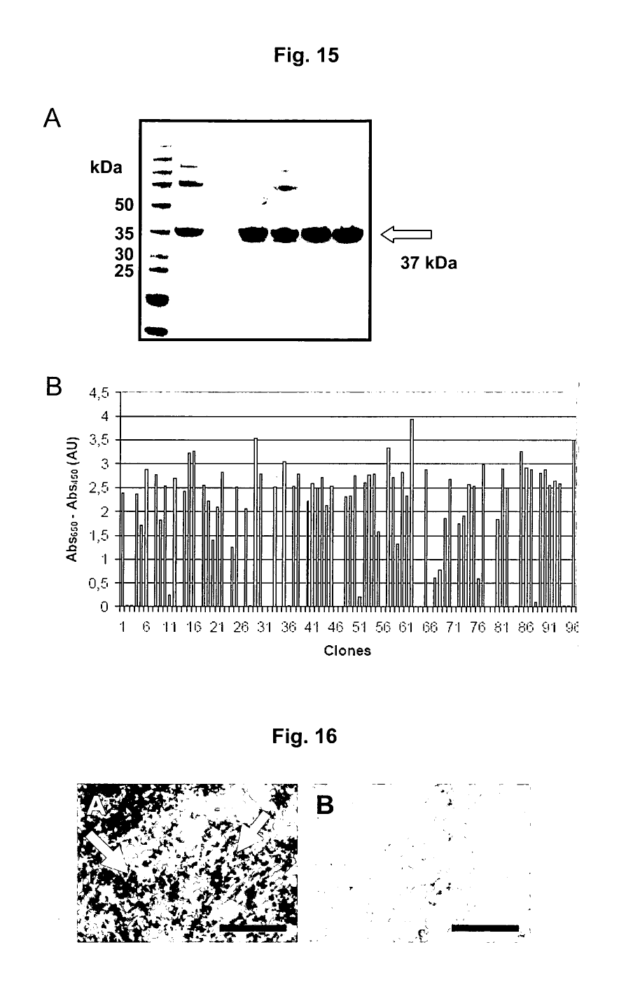

Almost the complete ANXA4 protein was cloned and expressed. FIG. 15A shows an SDS-PAGE analysis of the purified recombinant ANXA4 protein (band at 37 kDa). Phage display selections against this protein resulted in numerous clones expressing scFv antibodies specific to ANXA4 protein as shown by ELISA on the coated recombinant protein (see FIG. 15B). The best clone E11 against ANXA4 protein was used in an immunohistochemical analysis, which showed positive staining of tumor cells (also those cells located around the tumor blood vessels) on tissue sections of human kidney tumors (see FIG. 16A). The negative control, for which sections from the same tissue were submitted to the same staining protocol only omitting the scFv, showed no positive staining (see FIG. 16B). These results demonstrate that human monoclonal antibodies against ANXA4 protein can be produced and used for the detection of this antigen in human cancer tissues. The immunohistochemical results indicate that this antigen is expressed at high levels in human tumors (also around the neovasculature).

### Example 9

### Introduction

To further study the antigen UPF0378 family protein KIAA0100, we have cloned and expressed a recombinant fragment of this protein in order to use it as antigen for the selection of antibodies by phage display.

### Materials and Methods

A recombinant protein fragment corresponding to the amino acid sequence positions 755-968 of UPF0378 family protein KIAA0100 (SEQ ID NO: 96) was cloned and expressed as described in example 2 for use as antigen for biopanning experiments.

### Results and Discussion

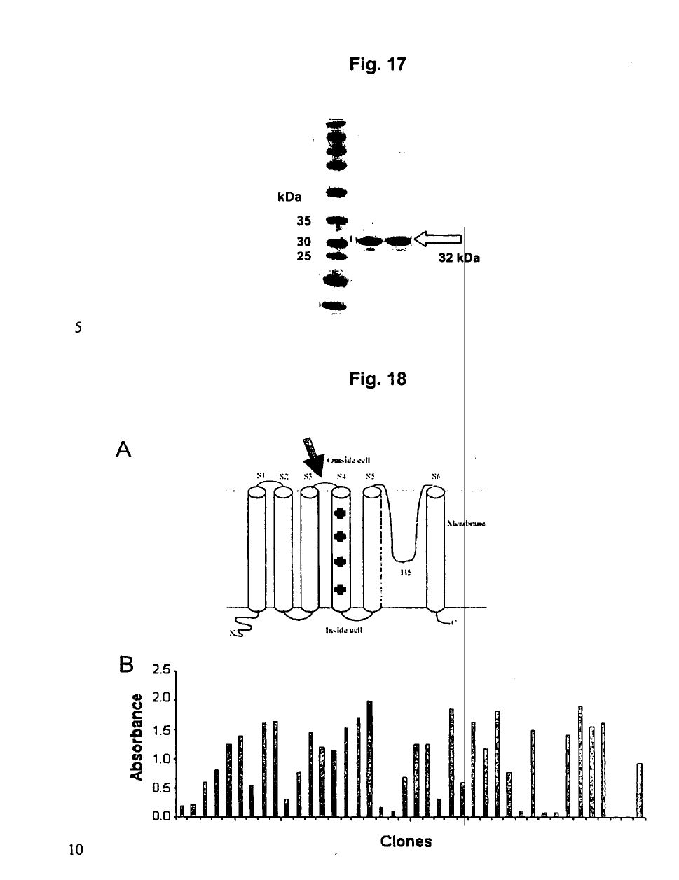

The UPF0378 family protein KIAA0100 fragment corresponding to the amino acid sequence positions 755-968 was cloned and expressed. FIG. 17 shows an SDS-PAGE analysis of the purified recombinant protein (band at 32 kDa). This result indicates that UPF0378 family protein KIAA0100 fragments can be prepared in order to use them as antigen in antibody selections. Such antibodies could be used for a further validation of this antigen as a vascular tumor marker similar to example 2.

### Example 10

### Introduction

For evaluating the usefulness of Potassium voltage-gated channel subfamily H member 1 as tumor marker, human monoclonal antibodies were produced against a synthetic peptide corresponding to the amino acid sequence of an extracellular loop of this membrane protein. These antibodies were tested in ELISA, immunohistochemistry, FACS and immunocytochemistry experiments.

### Materials and Methods

A synthetic peptide corresponding to the second extracellular loop (amino acid sequence positions 316-349; see FIG. 18A for a graphic representation) of Potassium voltage-gated channel subfamily H member 1 (SEQ ID NO: 98) was used as antigen for biopanning experiments. Antibodies in single chain Fv format (scFv) were selected from the ETH-2-Gold phage display library against the biotinylated peptide according to the procedure described in Silacci et al., Proteomics. 2005 June; 5(9):2340-50. ELISA screening, immunohistochemical staining and affinity maturation of selected antibodies were performed as described in Example 2. FACS and immunocytochemical staining of cultured cells were performed according to standard procedures using the selected scFv as primary, mouse anti-c-myc (clone 9E10) as secondary and FITC-labeled anti-mouse Ig as tertiary antibody.

### Results

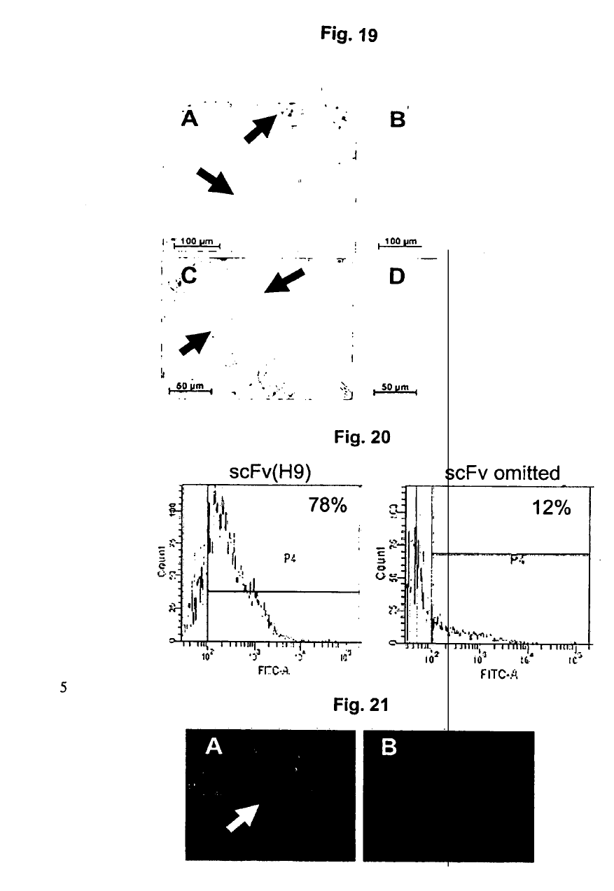

Phage display selections against the biotinylated Potassium voltage-gated channel subfamily H member 1 peptide (see above and FIG. 18A) resulted in numerous clones expressing binding scFv antibodies as tested by ELISA on the coated peptide (data not shown). The best clone H9 against was further affinity matured to obtain an antibody with higher affinity (see ELISA results in FIG. 18B). The selected anti-Potassium voltage-gated channel subfamily H member 1 antibody H9 was used in an immunohistochemical analysis, which showed positive staining of tumor cell membranes on tissue sections of human kidney tumors (see FIG. 19A) and of human lung tumors (see FIG. 19C). The negative controls, for which sections from the same tissues were submitted to the same staining protocol only omitting the scFv, showed no positive staining (see FIGS. 19B and 19D). Further studies on HeLa cells (a human cervix carcinoma cell line) showed that the scFv antibody H9 can recognize these cells in FACS (see FIG. 20) and gives a positive staining of the cell membranes in immunocytochemistry (see FIG. 21). These results demonstrate that human monoclonal antibodies against Potassium voltage-gated channel subfamily H member 1 can be produced and that the antigen is expressed on the tumor cell membrane of human cancer tissues (also those tumor cells which are in close proximity to the neovasculature) and on the membranes of human tumor cell lines. Thus, it is likely that this antigen is a suitable target for vascular tumor targeting approaches.

The following Table is a list of the amino acid sequences of the vascular tumor markers identified according to the invention. The partial amino acid sequences identified in Example 1 are illustrated in bold letters. The SEQ ID NOs therein correspond to the appended sequence listing which is part of the disclosure of the present invention.

| | | |
|---|---|---|
| | | Swiss Prot. |
| # | Name | Acc. No. |

| | | |
|---|---|---|
| 1 | Periostin [precursor] (+ isoforms) | Q15063 |

SEQ ID NO: 1

MIPFLPMFSLLLLLIVNPINANNHYDKILAHSRIRGRDQGPNVCALQQIL
GTKKKYFSTCKNWYKKSICGQKTTVLYECCPGYMRMEGMKGCPAVLPIDH
VYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYP
NGVVTVNCARIIHGNQIATNGVVHVIDRVLTQIGTSIQDFIEAEDDLSSF
RAAAITSDILEALGRDGHFTLFAPTNEAFEKLPRGVLERFMGDKVASEAL
MKYHILNTLQCSESIMGGAVFETLEGNTIEIGCDGDSITVNGIKMVNKKD
IVTNNGVIHLIDQVLIPDSAKQVIELAGKQQTTFTDLVAQLGLASALRPD
GEYTLLAPVNNAFSDDTLSMVQRLLKLILQNHILKVKVGLNELYNGQILE
TIGGKQLRVFVYRTAVCIENSCMEKGSKQGRNGAIHIFREIIKPAEKSLH
EKLKQDKRFSTFLSLLEAADLKELLTQPGDWTLFVPTNDAFKGMTSEEKE
ILIRDKNALQNIILYHLTPGVFIGKGFEPGVTNILKTTQGSKIFLKEVN
DTLLVNELKSKESDIMTTNGVIHVVDKLLYPADTPVGNDQLLEILNKLIK
YIQIKFVRGSTFKEIPVTVYTTKIITKVVEPKIKVIEGSLQPIIKTEGPT
LTKVKIEGEPEFRLIKEGETITEVIHGEPIIKKYTKIIDGVPVEITEKET
REERIITGPEIKYTRISTGGGETEETLKKLLQEEVTKVTKFIEGGDGHLF
EDEEIKRLLQGDTPVRKLQANKKVQGSRRRLREGRSQ

SEQ ID NO: 3

MIPFLPMFSLLLLLIVNPINANNHYDKILAHSRIRGRDQGPNVCALQQIL
GTKKKYFSTCKNWYKKSICGQKTTVLYECCPGYMRMEGMKGCPAVLPIDH
VYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYP
NGVVTVNCARIIHGNQIATNGVVHVIDRVLTQIGTSIQDFIEAEDDLSSF
RAAAITSDILEALGRDGHFTLFAPTNEAFEKLPRGVLERFMGDKVASEAL
MKYHILNTLQCSESIMGGAVFETLEGNTIEIGCDGDSITVNGIKMVNKKD
IVTNNGVIHLIDQVLIPDSAKQVIELAGKQQTTFTDLVAQLGLASALRPD
GEYTLLAPVNNAFSDDTLSMDQRLLKLILQNHILKVKVGLNELYNGQILE
TIGGKQLRVFVYRTAVCIENSCMEKGSKQGRNGAIHIFREIIKPAEKSLH
EKLKQDKRFSTFLSLLEAADLKELLTQPGDWTLFVPTNDAFKGMTSEEKE
ILIRDKNALQNIILYHLTPGVFIGKGFEPGVTNILKTTQGSKIFLKEVND
TLLVNELKSKESDIMTTNGVIHVVDKLLYPADTPVGNDQLLEILNKLIKY
IQIKFVRGSTFKEIPVTVYKPIIKKYTKIIDGVPVEITEKETREERIITG
PEIKYTRISTGGGETEETLKKLLQEEVTKVTKFIEGGDGHLFEDEEIKRL
LQGDTPVRKLQANKKVQGSRRRLREGRSQ

SEQ ID NO: 5

MIPFLPMFSLLLLLIVNPINANNHYDKILAHSRIRGRDQGPNVCALQQIL
GTKKKYFSTCKNWYKKSICGQKTTVLYECCPGYMRMEGMKGCPAVLPIDH
VYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYP
NGVVTVNCARIIHGNQIATNGVVHVIDRVLTQIGTSIQDFIEAEDDLSSF
RAAAITSDILEALGRDGHFTLFAPTNEAFEKLPRGVLERFMGDKVASEAL
MKYHILNTLQCSESIMGGAVFETLEGNTIEIGCDGDSITVNGIKMVNKKD
IVTNNGVIHLIDQVLIPDSAKQVIELAGKQQTTFTDLVAQLGLASALRPD
GEYTLLAPVNNAFSDDTLSMDQRLLKLILQNHILKVKVGLNELYNGQILE
TIGGKQLRVFVYRTAVCIENSCMEKGSKQGRNGAIHIFREIIKPAEKSLH
EKLKQDKRFSTFLSLLEAADLKELLTQPGDWTLFVPTNDAFKGMTSEEKE
ILIRDKNALQNIILYHLTPGVFIGKGFEPGVTNILKTTQGSKIFLKEVND
TLLVNELKSKESDIMTTNGVIHVVDKLLYPADTPVGNDQLLEILNKLIKY
IQIKFVRGSTFKEIPVTVYRPTLTKVKIEGEPEFRLIKEGETITEVIHGE
PTIKKYTKIIDGVPVEITEKETREERIITGPEIKYTRISTGGGETEETLK
KLLQEEVTKVTKFIEGGDGHLFEDEEIKRLLQGDTPVRKLQANKKVQGSR
RRLREGRSQ

SEQ ID NO: 7

MIPFLPMFSLLLLLIVNPINANNHYDKILAHSRIRGRDQGPNVCALQQIL
GTKKKYFSTCKNWYKKSICGQKTTVLYECCPGYMRMEGMKGCPAVLPIDH
VYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYP
NGVVTVNCARIIHGNQIATNGVVHVIDRVLTQIGTSIQDFIEAEDDLSSF
RAAAITSDILEALGRDGHFTLFAPTNEAFEKLPRGVLERFMGDKVASEAL
MKYHILNTLQCSESIMGGAVFETLEGNTIEIGCDGDSITVNGIKMVNKKD
IVTNNGVIHLIDQVLIPDSAKQVIELAGKQQTTFTDLVAQLGLASALRPD
GEYTLLAPVNNAFSDDTLSMDQRLLKLILQNHILKVKVGLNELYNGQILE
TIGGKQLRVFVYRTAVCIENSCMEKGSKQGRNGAIHIFREIIKPAEKSLH
EKLKQDKRFSTFLSLLEAADLKELLTQPGDWTLFVPTNDAFKGMTSEEKE
ILIRDKNALQNIILYHLTPGVFIGKGFEPGVTNILKTTQGSKIFLKEVND
TLLVNELKSKESDIMTTNGVIHVVDKLLYPADTPVGNDQLLEILNKLIKY
IQIKFVRGSTFKEIPVTVYTTKIITKVVEPKIKVIEGSLQPIIKTEGPTL
TKVKIEGEPEFRLIKEGETITEVIHGEPIIKKYTKIIDGVPVEITEKETR
EERIITGPEIKYTRISTGGGETEETLKKLLQEDTPVRKLQANKKVQGSRR
RLREGRSQ

SEQ ID NO: 9

MIPFLPMFSLLLLLIVNPINANNHYDKILAHSRIRGRDQGPNVCALQQIL
GTKKKYFSTCKNWYKKSICGQKTTVLYECCPGYMRMEGMKGCPAVLPIDH
VYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYP
NGVVTVNCARIIHGNQIATNGVVHVIDRVLTQIGTSIQDFIEAEDDLSSF
RAAAITSDILEALGRDGHFTLFAPTNEAFEKLPRGVLERFMGDKVASEAL
MKYHILNTLQCSESIMGGAVFETLEGNTIEIGCDGDSITVNGIKMVNKKD
IVTNNGVIHLIDQVLIPDSAKQVIELAGKQQTTFTDLVAQLGLASALRPD
GEYTLLAPVNNAFSDDTLDMVQRLLKLILQNHILKVKVGLNELYNGQILE
TIGGKQLRVFVYRTAVCIENSCMEKGSKQGRNGAIHIFREIIKPAEKSLH
EKLKQDKRFSTFLSLLEAADLKELLTQPGDWTLFVPTNDAFKGMTSEEKE
ILIRDKNALQNIILYHLTPGVFIGKGFEPGVTNILKTTQGSKIFLKEVND
TLLVNELKSKESDTMTTNGVTHVVDKLLYPADTPVGNDQLLEILNKLIKY
IQIKFVRGSTFKEIPVTVYGPEIKYTRISTGGGETEETLKKLLQEDTPVR
KLQANKKVQGSRRRLREGRSQ

SEQ ID NO: 11

MIPFLPMFSLLLLLIVNPINANNHYDKILAHSRIRGRDQGPNVCALQQIL
GTKKKYFSTCKNWYKKSICGQKTTVLYECCPGYMRMEGMKGCPAVLPIDH
VYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYP
NGVVTVNCARIIHGNQIATNGVVHVIDRVLTQIGTSIQDFIEAEDDLSSF
RAAAITSDILEALGRDGHFTLFAPTNEAFEKLPRGVLERFMGDKVASEAL
MKYHILNTLQCSESIMGGAVFETLEGNTIEIGCDGDSITVNGIKMVNKKD
IVTNNGVIHLIDQVLIPDSAKQVIELAGKQQTTFTDLVAQLGLASALRPD
GEYTLLAPVNNAFSDDTLDMVQRLLKLILQNHILKVKVGLNELYNGQILE
TIGGKQLRVFVYRTAVCIENSCMEKGSKQGRNGAIHIFREIIKPAEKSLH
EKLKQDKRFSTFLSLLEAADLKELLTQPGDWTLFVPTNDAFKGMTSEEKE
ILIRDKNALQNIILYHLTPGVFIGKGFEPGVTNILKTTQGSKIFLKEVND
TLLVNELKSKESDIMTTNGVIHVVDKLLYPADTPVGNDQLLEILNKLIKY
IQIKFVRGSTFKEIPVTVYKPIIKKYTKIIDGVPVEITEKETREERIITG
PEIKYTRISTGGGETEETLKKLLQEEDTPVRKLQANKKVQGSRRRLREGR
SQ

SEQ ID NO: 13

MIPFLPMFSLLLLLIVNPINANNHYDKILAHSRIRGRDQGPNVCALQQIL
GTKKKYFSTCKNWYKKSICGQKTTVLYECCPGYMRMEGMKGCPAVLPIDH
VYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYP
NGVVTVNCARIIHGNQIATNGVVHVIDRVLTQIGTSIQDFIEAEDDLSSF
RAAAITSDILEALGRDGHFTLFAPTNEAFEKLPRGVLERFMGDKVASEAL
MKYHILNTLQCSESIMGGAVFETLEGNTIEIGCDGDSITVNGIKMVNKKD
IVTNNGVIHLIDQVLIPDSAKQVIELAGKQQTTFTDLVAQLGLASALRPD
GEYTLLAPVNNAFSDDTLDMVQRLLKLILQNHILKVKVGLNELYNGQILE
TIGGKQLRVFVYRTAVCIENSCMEKGSKQGRNGAIHIFREIIKPAEKSLH
EKLKQDKRFSTFLSLLEAADLKELLTQPGDWTLFVPTNDAFKGMTSEEKE
ILIRDKNALQNIILYHLTPGVFIGKGFEPGVTNILKTTQGSKIFLKEVND
TLLVNELKSKESDIMTTNGVIHVVDKLLYPADTPVGNDQLLEILNKLIKY
IQIKFVRGSTFKEIPVTVYGPEIKYTRISTGGGETEETLKKLLQEEVTKV
TKFIEGGDGHLFEDEEIKRLLQGDTPVRKLQANKKVQGSRRRLREGRSQ

SEQ ID NO: 15

MIPFLPMFSLLLLLIVNPINANNHYDKILAHSRIRGRDQGPNVCALQQIL
GTKKKYFSTCKNWYKKSICGQKTTVLYECCPGYMRMEGMKGCPAVLPIDH
VYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYP
NGVVTVNCARIIHGNQIATNGVVHVIDRVLTQIGTSIQDFIEAEDDLSSF
RAAAITSDILEALGRDGHFTLFAPTNEAFEKLPRGVLERFMGDKVASEAL
MKYHILNTLQCSESIMGGAVFETLEGNTIEIGCDGDSITVNGIKMVNKKD
IVTNNGVIHLIDQVLIPDSAKQVIELAGKQQTTFTDLVAQLGLASALRPD
GEYTLLAPVNNAFSDDTLDMVQRLLKLILQNHILKVKVGLNELYNGQILE
TIGGKQLRVFVYRTAVCIENSCMEKGSKQGRNGAIHIFREIIKPAEKSLH
EKLKQDKRFSTFLSLLEAADLKELLTQPGDWTLFVPTNDAFKGMTSEEKE
ILIRDKNALQNIILYHLTPGVFIGKGFEPGVTNILKTTQGSKIFLKEVND
TLLVNELKSKESDIMTTNGVIHVVDKLLYPADTPVGNDQLLEILNKLIKY
IQIKFVRGSTFKEIPVTVYRPTLTKVKIEGEPEFRLIKEGETITEVIHGE
PIIKKYTKIIDGVPVEITEKETREERIITGPEIKYTRISTGGGETEETLK
KLLQEEVTKVTKFIEGGDGHLFEDEEIKRLLQGDTPVRKLQANKKVQGSR
RRLREGRSQ

| | | |
|---|---|---|
| | Putative G-protein coupled | |
| 2 | receptor 42 (+ isoforms) | O15529 |

SEQ ID NO: 17

MDTGPDQSYFSGNHWFVFSVYLLTFLVGLPLNLLALVVFVGKLRCRPVAV
DVLLLNLTASDLLLLLFLPFRMVEAANGMHWPLPFILGPLSGFIFFTTIY
LTALFLAAVSIERFLSVAHPLWYKTRPRLGQAGLVSVACWLLASAHCSVV
YVIEFSGDISHSQGTNGTCYLEFWKDQLAILLPVRLEMAVVLFVVPLIIT
SYCYSRLVWILGRGGSHRRQRRVAGLVAATLLNFLVCFGPYNVSHVVGYI
CGESPVWRIYVTLLSTLNSCVDPFVYYFSSSGFQADFHELLRRLCGLWGQ
WQQESSMELKEQKGGEEQRADRPAERKTSEHSQGCGTGGQVACAEN

SEQ ID NO: 19

MDTGPDQSYFSGNHWFVFSVYLLTFLVGLPLNLLALVVFVGKLQRRPVAV
DVLLLNLTASDLLLLLFLPFRMVEAANGMHWPLPFILCPLSGFIFFTTIY
LTALFLAAVSIERFLSVAHPLWYKTRPRLGQAGLVSVACWLLASAHCSVV
YVIEFSGDISHSQGTNGTCYLEFRKDQLAILLPVRLEMAVVLFVVPLIIT
SYCYSRLVWILGRGGSHRRQRRVAGLLAATLLNFLVCFGPYNVSHVVGYI
CGESPAWRIYVTLLSTLNSCVDPFVYYFSSSGFQADFHELLRRLCGLWGQ
WQQESSMELKEQKGGEEQRADRPAERKTSEHSQGCGTGGQVACAES

| | | |
|---|---|---|
| | Solute carrier family 2, | |
| | facilitated glucose transporter | |
| 3 | member 1 | P11166 |

SEQ ID NO: 21

MEPSSKKLTGRLMLAVGGAVLGSLQFGYNTGVINAPQKVIEEFYNQTWVH
RYGESILPTTLTTLWSLSVAIFSVGGMIGSFSVGLFVNRFGRRNSMLMMN
LLAFVSAVLMGFSKLGKSFEMLILGRFIIGVYCGLTTGFVPMYVGEVSPT
AFRGALGTLHQLGIVVGILIAQVFGLDSIMGNKDLWPLLLSIIFIPALLQ
CIVLPFCPESPRFLLINRNEENRAKSVLKKLRGTADVTHDLQEMKEESRQ
MMREKKVTILELFRSPAYRQPILIAVVLQLSQQLSGINAVFYYSTSIFEK
AGVQQPVYATIGSGIVNTAFTVVSLFVVERAGRRTLHLIGLAGMAGCAIL
MTIALALLEQLPWMSYLSIVAIFGFVAFFEVGPGPIPWFIVAELFSQGPR
FAAIAVAGFSNWTSNFIVGMCFQYVEQLCGPYVFIIFTVLLVLFFIFTYF
KVPETKGRTFDEIASGFRQGGASQSDKTPEELFHPLGADSQV

| | | |
|---|---|---|
| 4 | Versican core rotein recursor | P13611 |

SEQ ID NO: 23

MFINIKSILWMCSTLIVTHALHKVKVGKSPPVRGSLSGKVSLPCHFSTMP
TLPPSYNTSEFLRIKWSKIEVDKNGKDLKETTVLVAQNGNIKIGQDYKGR
VSVPTHPEAVGDASLTVVKLLASDAGLYRCDVMYGIEDTQDTVSLTVDGV
VFHYRAATSRYTLNFEAAQKACLDVGAVIATPEQLFAAYEDGFEQCDAGW
LADQTVRYPIRAPRVGCYGDKMGKAGVRTYGFRSPQETYDVYCYVDHLDG
DVFHLTVPSKFTEEEAAKECENQDARLATVGELQAAWRNGFDQCDYGWLS
DASVRHPVTVARAQCGGGLLGVRTLYRFENQTGFPPPDSRFDAYCFKPKE
ATTIDLSILETASPSLSKEPQMVSDRTTPIIPLVDELPVIPTEFPPVGNI
VSFEQKATVQPQAITDSLATKLPTPTGSTKKPWDMDDYSPSASGPLGKLD
ISEIKEEVLQSTTGVSHYATDSWDGVVEDKQTQESVTQIEQIEVGPLVTS
MEILKHIPSKEFPVTETPLVTARNILESKTEKKMVSTVSELVTTGHYGFT
LGEEDDEDRTLTVGSDESTLIFDQIPEVITVSKTSEDTIHTHLEDLESVS
ASTTVSPLIMPDNNGSSMDDWEERQTSGRITEEFLGKYLSTTPFPSQHRT
EIELFPYSGDKILVEGISTVIYPSLQTEMTHRRERTETLIPEMRTDTYTD
EIQEEITKSPFMGKTEEEVFSGMKLSTSLSEPIHVTESSVEMTKSFDFPT
LITKLSAEPTEVRDMEEDFTATPGTTKYDENITTVLLAHGTLSVEAATVS
KWSWDEDNTTSKPLESTEPSASSKLPPALLTTVGMNGKDKDIPSFTEDGA
DEFTLIPDSTQKQLEEVTDEDIAAHGKFTIRFQPTTSTGIAEKSTLRDST
TEEKVPPITSTEGQVYATMEGSALGEVEDVDLSKPVSTVPQFAHTSEVEG
LAFVSYSSTQEPTTYVDSSHTIPLSVIPKTDWGVLVPSVPSEDEVLGEPS
QDILVIDQTRLEATISPETMRTTKITEGTTQEEFPWKEQTAEKPVPALSS
TAWTPKEAVTPLDEQEGDGSAYTVSEDELLTGSERVPVLETTPVGKIDHS
VSYPPGAVTEHKVKTDEVVTLTPRIGPKVSLSPGPEQKYETEGSSTTGFT
SSLSPFSTHITQLMEETTTEKTSLEDIDLGSGLFEKPKATELIEFSTIKV
TVPSDITTAFSSVDRLHTTSAFKPSSAITKKPPLIDREPGEETTSDMVII
GESTSHVPPTTLEDIVAKETETDIDREYFTTSSPPATQPTRPPTVEDKEA
FGPQALSTPQPPASTKFHPDINVYIIEVRENKTGRMSDLSVIGHPIDSES
KEDEPCSEETDPVHDLMAEILPEFPDIIEIDLYHSEENEEEEEECANATD
VTTTPSVQYINGKHLVTTVPKDPEAAEARRGQFESVAPSQNFSDSSESDT
HPFVIAKTELSTAVQPNESTETTESLEVTWKPETYPETSEHFSGGEPDVF
PTVPFHEEFESGTAKKGAESVTERDTEVGHQAHEHTEPVSLFPEESSGEI
AIDQESQKIAFARATEVTFGEEVEKSTSVTYTPTIVPSSASAYVSEEEAV
TLIGNPWPDDLLSTKESWVEATPRQVVELSGSSSIPITEGSGEAEEDEDT
MFTMVTDLSQRNTTDTLITLDTSRIITESFFEVPATTIYPVSEQPSAKVV
PTKFVSETDTSEWISSTTVEEKKRKEEEGTTGTASTFEVYSSTQRSDQLI
LPFELESPNVATSSDSGTRKSFMSLTTPTQSEREMTDSTPVFTETNTLEN
LGAQTTEHSSIHQPGVQEGLTTLPRSPASVFMEQGSGEAAADPETTTVSS
FSLNVEYAIQAEKEVAGTLSPHVETTFSTEPTGLVLSTVMDRVVAENITQ
TSREIVISERLGEPNYGAEIRGFSTGFPLEEDFSGDFREYSTVSHPIAKE
ETVMMEGSGDAAFRDTQTSPSTVPTSVHISHISDSEGPSSTMVSTSAFPW
EEFTSSAEGSGEQLVTVSSSVVPVLPSAVQKFSGTASSIIDEGLGEVGTV
NEIDRRSTILPTAEVEGTKAPVEKEEVKVSGTVSTNFPQTIEPAKLWSRQ
EVNPVRQEIESETTSEEQIQEEKSFESPQNSPATEQTIFDSQTFTETELK
TTDYSVLTTKKTYSDDKEMKEEDTSLVNMSTPDPDANGLESYTTLPEATE
KSHFFLATALVTESIPAEHVVTDSPIKKEESTKHFPKGMRPTIQESDTEL
LFSGLGSGEEVLPTLPTESVNFTEVEQINNTLYPHTSQVESTSSDKIEDF
NRMENVAKEVGPLVSQTDIFEGSGSVTSTTLIEILSDTGAEGPTVAPLPF
STDIGHPQNQTVRWAEETQTSRPQTITEQDSNKNSSTAEINETTTSSTDF
LARAYGFEMAKEFVTSAPKPSDLYYEPSGEGSGEVDIVDSFHTSATTQAT
RQESSTTFVSDGSLEKHPEVPSAKAVTADGFPTVSVMLPLHSEQNKSSPD
PTSTLSNTVSYERSTDGSFQDRFREFEDSTLKFNRKKPTENIIIDLDKED
KDLILTITESTILEILPELTSDKNTTIDIDHTKPVYEDILGMQTDIDTEV
PSEPHDSNDESNDDSTQVQEIYEAAVNLSLTEETFEGSADVLASYTQATH
DESMTYEDRSQLDHMGFHFTTGIPAPSTETELDVLLPTATSLPIPRKSAT
VIPEIEGIKAEAKALDDMFESSTLSDGQAIADQSEIIPTLGQFERTQEEY
EDKKHAGPSFQPEFSSGAEEALVDHTPYLSIATTHLMDQSVTEVPDVMEG
SNPPYYTDTTLAVSTFAKLSSQTPSSPLTIYSGSEASGHTEIPQPSALPG
IDVGSSVMSPQDSFKEIHVNIEATFKPSSEEYLHITEPPSLSPDTKLEPS
EDDGKPELLEEMEASPTELIAVEGTEILQDFQNKTDGQVSGEAIKMFPTI
KTPEAGTVITTADEIELEGATQWPHSTSASATYGVEAGVVPWLSPQTSER
PTLSSSPEINPETQAALIRGQDSTIAASEQQVAARILDSNDQATVNPVEF
NTEVATPPFSLLETSNETDFLIGINEESVEGTAIYLPGPDRCKMNPCLNG
GTCYPTETSYVCTCVPGYSGDQCELDFDECHSNPCRNGATCVDGFNTFRC
LCLPSYVGALCEQDTETCDYGWHKFQGQCYKYFAHRRTWDAAERECRLQG
AHLTSILSHEEQMFVNRVGHDYQWIGLNDKMFEHDFRWTDGSTLQYENWR
PNQPDSFFSAGEDCVVIIWHENGQWNDVPCNYHLTYTCKKGTVACGQPPV
VENAKTFGKMKPRYEINSLIRYHCKDGFIQRHLPTIRCLGNGRWAIPKIT
CMNPSAYQRTYSMKYFKNSSSAKDNSINTSKHDHRWSRRWQESRR

| | | |
|---|---|---|
| 5 | CEACAM3 (+ isoforms) | Q6UY47 |

SEQ ID NO: 25

MGPPSACPHRECIPWQGLLLTASLLTFWNAPTTAWLFIASAPFEVAEGEN
VHLSVVYLPENLYSYGWYKGKTVEPNQLIAAYNIDTHVRTPGPAYSGRET
ISPSGDLHFQNVTLEDTGYYNLQVTYRNSQIEQASHHLRVYESVAQPSIQ
ASSTTVTEKGSVVLTCHTNNTGTSFQWIFNNQRLQVTKRMKLSWFNHVLT
ICPIRQEDAGEYQCEVSNPVSSNRSDPLKLTVKYDNTLGILIGVLVGSLL
VAALVCFLLLRKTGRASDQSDFREQQPPASTPGHGPSDSSIS

SEQ ID NO: 27

MGPPSACPHRECIPWQGLLLTASLLTFWNAPTTAWLFIASAPFEVAEGEN
VHLSVVYLPENLYSYGWYKGKTVEPNQLIAAYNIDTHVRTPGPAYSGRET
ISPSGDLHFQNVTLEDTGYYNLQVTYRNSQIEQASHHLRVYESVAQPSIQ
ASSTTVTEKGSVVLTCHTNNTGTSFQWIFNNQRLQVTKRMKLSWFNHVLT
ICPIRQEDAGEYQCEVSNPVSSNRSDPLKLTVKSDDNTLGILIGVLVGSL
LVAALVCFLLLRKTGRASDQSDFREQQPPASTPGHGPSDSSIS

SEQ ID NO: 29

MGPPSACPHRECIPWQGLLLTASLLTFWNAPTTAWLFIASAPFEVAEGEN
VHLSVVYLPENLYSYGWYKGKTVEPNQLIAAYNIDTHVRTPGPAYSGRET
ISPSGDLHFQNVTLEDTGYYNLQVTYRNSQIEQASHHLRVYESVAQPSIQ
ASSTTVTEKGSVVLTCHTNNTGTSFQWIFNNQRLQVTKRMKLSWFNHVLT
ICPIRQEDAGEYQCEVSNPVSSNRSDPLKLTVKCE

| | | |
|---|---|---|
| 6 | Fibromodulin | Q81V47 |

SEQ ID NO: 31

MQWTSLLLLAGLFSLSQAQYEDDPHWWFHYLRSQQSTYYDPYDPYPYETY
EPYPYGVDEGPAYTYGSPSPPDPRDCPQECDCPPNFPTAMYCDNRNLKYL
PFVPSRMKYVYFQNNQITSIQEGVFDNATGLLWIALHGNQITSDKVGRKV
FSKLRHLERLYLDHNNLTRMPGPLPRSLRELHLDHNQISRVPNNALEGLE
NLTALYLQHNEIQEVGSSMRGLRSLILLDLSYNHLRKVPDGLPSALEQLY
MEHNNVYTVPDSYFRGAPKLLYVRLSHNSLTNNGLASNTFNSSSLLELDL
SYNQLQKIPPVNTNLENLYLQGNRINEFSISSFCTVVDVVNFSKLQVLRL
DGNEIKRSANPADAPLCLRLASLIEI

| | | |
|---|---|---|
| 7 | Peroxidasin homolog [Fragment] | Q92626 |

SEQ ID NO: 33

SRPWWLRASERPSAPSAMAKRSRGPGRRCLLALVLFCAWGTLAVVAQKPG
AGCPSRCLCFRTTVRCMHLLLEAVPAVAPQTSILDLRFNRIREIQPGAFR
RLRNLNTLLLNNNQIKRIPSGAFEDLENLKYLYLYKNEIQSIDRQAFKGL
ASLEQLYLHFNQIETLDPDSFQHLPKLERLFLHNNRITHLVPGTFNHLES
MKRLRLDSNTLHCDCEILWLADLLKTYAESGNAQAAAICEYPRRIQGRSV
ATITPEELNCERPRITSEPQDADVTSGNTVYFTCRAEGNPKPEIIWLRNN
NELSMKTDSRLNLLDDGTLMIQNTQETDQGIYQCMAKNVAGEVKTQEVTL
RYFGSPARPTFVIQPQNTEVLVGESVTLECSATGHPPPRISWTRGDRTPL
PVDPRVNITPSGGLYIQNVVQGDSGEYACSATNNIDSVHATAFIIVQALP
QFTVTPQDRVVIEGQTVDFQCEAKGNPPPVIAWTKGGSQLSVDRRHLVLS
SGTLRISGVALHDQGQYECQAVNIIGSQKVVAHLTVQPRVTPVFASIPSD
TTVEVGANVQLPCSSQGEPEPAITWNKDGVQVTESGKFHISPEGFLTIND
VGPADAGRYECVARNTIGSASVSMVLSVNVPDVSRNGDPFVATSIVEAIA
TVDRAINSTRTHLFDSRPRSPNDLLALFRYPRDPYTVEQARAGEIFERTL
QLIQEHVQHGLMVDLNGTSYHYNDLVSPQYLNLIANLSGCTAHRRVNNCS
DMCFHQKYRTHDGTCNNLQHPMWGASLTAFERLLKSVYENGFNTPRGINP
HRLYNGHALPMPRLVSTTLIGTETVTPDEQFTHMLMQWGQFLDHDLDSTV
VALSQARFSDGQHCSNVCSNDPPCFSVMIPPNDSRARSGARCMFFVRSSP
VCGSGMTSLLMNSVYPREQINQLTSYIDASNVYGSTEHEARSIRDLASHR
GLLRQGIVQRSGKPLLPFATGPPTECMRDENESPIPCFLAGDHRANEQLG
LTSMHTLWFREHNRIATELLKLNPHWDGDTIYYETRKIVGAEIQHITYQH
WLPKILGEVGMRTLGEYHGYDPGINAGIFNAFATAAFRFGHTLVNPLLYR
LDENGQPIAQDHLPLHKAFFSPFRIVNEGGIDPLLRGLFGVAGKMRVPSQ
LLNTELTERLFSMAHTVALDLAAINIQRGRDHGIPPYHDYRVYCNLSAAH
TFEDLKNEIKNPEIREKLKRLYGSTLNIDLFPALVVEDLVPGSRLGPTLM
CLLSTQFKRLRDGDRLWYENPGVFSPAQLTQIKQTSLARILCDNADNITR
VQSDVFRVAEFPHGYGSCDEIPRVDLRVWQDCCEDCRTRGQFNAFSYHFR
GRRSLEFSYQEDKPTKKTRPRKIPSVGRQGEHLSNSTSAFSTRSDASGTN
DFREFVLEMQKTITDLRTQIKKLESRLSTTECVDAGGESHANNTKWKKDA
CTICECKDGQVTCFVEACPPATCAVPVNIPGACCPVCLQKRAEEKP

| | Probable G-protein coupled receptor | |
|---|---|---|
| 8 | 37 [precursor] | O15354 |

SEQ ID NO: 35

NRAPGALLARMSRLLLLLLLKVSASSALGVAPASRNETCLGESCAPTVIQ
RRGRDAWGPGNSARDVLRARAPREEQGAAFLAGPSWDLPAAPGRDPAAGR
GAEASAAGPPGPPTRPPGPWRWKGARGQEPSETLGRGNPTALQLFLQISE
EEEKGPRGAGISGRSQEQSVKTVPGASDLFYWPRRAGKLQGSHHKPLSKT
ANGLAGHEGWTIALPGRALAQNGSLGEGIHEPGGPRRGNSTNRRVRLKNP
FYPLTQESYGAYAVMCLSVVIFGTGIIGNLAVMCIVCHNYYMRSISNSLL
ANLAFWDFLIIFFCLPLVIFHELTKKWLLEDFSCKIVPYIEVASLGVTTF
TLCALCIDRFRAATNVQMYYEMIENCSSTTAKLAVIWVGALLLALPEVVL
RQLSKEDLGFSGRAPAERCIIKISPDLPDTIYVLALTYDSARLWWYFGCY
FCLPTLFTITCSLVTARKIRKAEKACTRGNKRQIQLESQMNCTVVALTIL
YGFCIIPENICNIVTAYMATGVSQQTMDLLNIISQFLLFFKSCVTPVLLF
CLCKPFSRAFMECCCCCCEECIQKSSTVTSDDNDNEYTTELELSPFSTIR
REMSTFASVGTHC

| | | |
|---|---|---|
| 9 | Protein sidekick-1 [precursor] | Q8TEN9 |

SEQ ID NO: 37

PEMTGVTVSGLTPARTYQFRVCAVNEVGRGQYSAETSRLMLPEEPPSAPP
KNIVASGRTNQSIMVQWQPPPETEHNGVLRGYILRYRLAGLPGEYQQRNI
TSPEVNYCLVTDLIIWTQYEIQVAAYNGAGLGVFSRAVTEYTLQGVPTAP
PQNVQTEAVNSTTIQFLWNPPPQQFINGINQGYKLLAWPADAPSAVTVVT
IAPDFHGVHHGHITNLKKFTAYFTSVLCFTTPGDGPPSTPQLVWTQEDKP
GAVGHLSFTEILDTSLKVSWQEPLEKNGIITGYQISWEVYGRNNSRLTHT
LNSTTHEYKIQGLSSLTTYTIDVAAVTAVGTGLVTSSTISSGVPPDLPGA
PSNLVISNISPRSATLQFRPGYDGKTSISRWIVEGQVGAIGDEEEWVTLY
EEENEPDAQMLEIPNLTPYTHYRFRMKQVNIVGPSPYSPSSRVIQTLQAP
PDVAPTSVTVRTASETSLRLRWVPLPDSQYNGNPESVGYRIKYWRSDLQS
SAVAQVVSDRLEREFTIEELEEWMEYELQMQAFNAVGAGPWSEVVRGRTR
ESVPSAAPENVSAEAVSSTQILLTWTSVPEQDQNGLILGYKILFRAKDLD
PEPRSHIVRGNHTQSALLAGLRKFVLYELOVLAFTRIGNGVPSTPLILER
TKDDAPGPPVRLVFPEVRLTSVRIVWQPPEEPNGIILGYQIAYRLASSSP
HTFTTVEVGATVRQFTATDLAPESAYIFRLSAKTRQGWGEPLEATVITTE
KRERPAPPRELLVPQAEVTARSLRLQWVPGSDGASPIRYFTMQVRELPRG
EWQTYSSSISHEATACVVDRLRPFTSYKLRLKATNDIGDSDFSSETEAVT
TLQDVPGEPPGSVSATPHTTSSVLIQWQPPRDESLNGLLQGYRIYYRELE
YEAGSGTEAKTLKNPIALRAELTDLKKYRRYEVIMTAYNIIGESPASAPV
EVFVGEAAPAMAPQNVQVTPLTASQLEVTWDPPPPESQNGNIQGYKIYYW
EADSQNETEKMKVLFLPEPVVRLKNLTSHTKYLVSISAFNAAGDGPKSDP
QQGRTHQAAPGAPSFLAFSEITSTTLNVSWGEPAAANGILQGYRVVYEPL
APVQGVSKVVTVEVRGNWQRWLKVRDLTKGVTYFFRVQARTITYGPELQA
NITAGPAEGSPGSPRDVLVTKSASELTLQWTEGHSGDTPTTGYVIEARPS
DEGLWDMFVKDIPRSATSYTLSLDKLRQGVTYEFRVVAVNEAGYGEPSNP
STAVSAQVEAPFYEEWWFLLVMALSSLIVILLVVFALVLHGQNKKYKNCS
TGKGISTMEESVTLDNGGFAALELSSRHLNVKSTFSKKNGTRSPPRPSPG
GLHYSDEDICNKYNGAVLTESVSLKEKSADASESEATDSDYEDALPKHSF
VNHYMSDPTYYNSWKRRAQGRAPAPHRYEAVAGSEAGAQLHPVITTQSAG
GVYTPAGPGARTPLTGFSSFV

| | | |
|---|---|---|
| | Alpha1A-voltage-dependent calcium | |
| 10 | channel | Q9NS88 |

SEQ ID NO: 39

MARFGDEMPARYGGCGSCAAAGVVVGSGGGRGAGGSRQGGQPGAQRMYKQ
SMAQRARTMALYNPIPVRQNCLTVNRSLFLFSEDNVVRKYAKKITEWPPF
EYMILATIIANCIVLALEQHLPDDDKTPMSERLDDTEPYFIGIFCFEAGI
KIIALGFAFHKGSYLRNGWNVMDFVVVLTGILATVGTEFDLRTLRAVRVL
RPLKLVSGIPSLQVVLKSIMKANIPLLQIGLLLFFAILIFAIIGLEFYMG
KFHTTCFEEGTDDIQGESPAPCGTEEPARTCPNGTKCQPYWEGPNNGITQ
FDNILFAVLTVFQCITMEGWTDLLYNSNDASGNTWNWLYFIPLIIIGSFF
MLNLVLGVLSGEFAKERERVENRRAFLKLRRQQQIERELNGYMEWISKAE
EVILAEDETDGEQRHPFDALRRTTIKKSKTDLLNPEEAEDQLADIASVGS
PFARASIKSAKLENSTFFHKKERRMRFYIRRMVKTQAFYWTVLSLVALNT
LCVAIVHYNQPEWLSDFLYYAEFIFLGLFMSEMFIKMYGLGTRPYFHSSF
NCFDCGVIIGSIFEVIWAVIKPGTSFGISVLRALRLLRIFKVTKYWASLR
NLVVSLLNSMKSIISLLFLLELFIVVFALLGMQLFGGQFNEDEGTPPTNF
DTFPAAIMTVFQILTGEDWNEVMYDGIKSQGGVQGGMVFSIYFIVLTLFG
NYTLLNVFLAIAVDNLANAQELTKDEQEEEEAANQKLALQKAKEVAEVSP
LSAANMSIAVKEQQKNQKPAKSVWEQRTSEMRKQNLLASREALYNEMDPD
ERWKAAYTRHLRPDMKTHLDRPLVVDPQENRNNNTNKSRAAEPTVDQRLG
QQRAEDFLRKQARYHDRARDPSGSAGLDARRPWAGSQEAELSREDPYGRE
SDHHAREGSLEQPGFWEGEAERGKAGDPHRRHVHRQGGSRESRSGSPRTG
ANGEHRRHRAHRSPGEEGPEDKAERRARHREGSRPARGGEGEGEGPDGGE
RRRRHRHGAPATYEGDARREDKERRHRRRKENQGSGVPVSGPNLSTTRPI
QQDLGRQDPPLAEDIDNMKNNKLATAESAAPHGSLGHAGLPQSPAKMGNS
TDPSPMLAIPAMATNPQNAASRRTPNNPGNPSNPGPPKTPENSLIVTNPS
GTQTNSAKTARKPDHTTVDIPPACPPPLNHTVVQVNKNANPDPLPKKEEE
KKEEEEDDRGEDGPKPMPPYSSMFILSTTNPLRRLCHYILNLRYFEMCIL
MVIAMSSIALAAEDPVQPNAPRNNVLRYFDYVFTGVFTFEMVIKMIDLGL
VLHQGAYFRDLWNILDFIVVSGALVAFAFTGNSKGKDINTIKSLRVLRVL
RPLKTIKRLPKLKAVFDCVVNSLKNVFNILIVYMLFMFIFAVVAVQLFKG
KFFHCTDESKEFEKDCRGKYLLYEKNEVKARDREWKKYEFHYDNVLWALL
TLFTVSTGEGWPQVLKHSVDATFENQGPSPGYRMEMSIFYVVYFVVFPFF
FVNIFVALIIITFQEQGDKMMEEYSLEKNERACIDFAISAKPLTRHMPQN
KQSFQYRMWQFVVSPPFEYTIMAMIALNTIVLMMKFYGASVAYENALRVF
NIVFTSLFSLECVLKVMAFGILNYFRDAWNIFDFVTVLGSITDILVTEFG
NNFINLSFLRLFRAARLIKLLRQGYTIRILLWTFVQSFKALPYVCLLIAM
LFFIYAIIGMQVFGNIGIDVEDEDSDEDEFQITEHNNFRTFFQALMLLFR
SATGEAWHNIMLSCLSGKPCDKNSGILTRECGNEFAYFYFVSFIFLCSFL
MLNLFVAVIMDNFEYLTRDSSILGPHHLDEYVRVWAEYDPAAWGRMPYLD
MYQMLRHMSPPLGLGKKCPARVAYKRLLRMDLPVADDNTVHFNSTLMALI
RTALDIKIAKGGADKQQMDAELRKEMMAIWPNLSQKTLDLLVTPHKSTDL
TVGKIYAAMMIMEYYRQSKAKKLQAMREEQDRTPLMFQRNEPPSPTQEGG
PGQNALPSTQLDPGGALMAHESGLKESPSWVTQRAQEMFQKTGTWSPEQG
PPTDMPNSQPNSQSVEMREMGRDGYSDSEHYLPMEGQGRAASMPRLPAEN
QRRRGRPRGNNLSTISDTSPMKRSASVLGPKARRLDDYSLERVPPEENQR
HHQRRRDRSHRASERSLGRYTDVDTGLGTDLSMTTQSGDLPSKERDQERG
RPKDRKHRQHHHHHHHHHHPPPPDKDRYAQERPDHGRARARDQRWSRSPS
EGREHMAHRQGSSSVSGSPAPSTSGTSTPRRGRRQLPQTPSTPRPHVSYS
PVIRKAGGSGPPQQQQQQQQQQQQQAVARPGRAATSGPRRYPGPTAEPLA
GDRPPTGGHSSGRSPRMERRVPGPARSESPRACRHGGARWPASGPHVSEG
PPGPRHHGYYRGSDYDEADGPGSGGGEEAMAGAYDAPPPVRHASSGATGR
SPRTPRASGPACASPSRHGRRLPNGYYPAHGLARPRGPGSRKGLHEPYSE
SDDDWC

| | | |
|---|---|---|
| 11 | EMILIN2 protein [fragment] | Q8N5L1 |

SEQ ID NO: 41

RGALVGAPRSARASCLRGRRPGRRQPCGRCPDPPRSGPGQAGRARCARDV
AAQTALAPRALALGAGAAGPGWRGAVPRRPAARVSRAAQRQEQELVRLHR
EQECELQPEPAPPRPSGPATAEDPGRRPVLPQRPPEERPPQPPGSTGVIA
ETGQAGPPAGAGAPGYPKSPPVASPGAPVPSLVSFASAGLTQKPFPSDGG
VVLFNLVLVNDGDVYNPSTGVFTAPYDGRYLITALTPERDAYVEAVLSVS
NASVAQLHTAGYRREFLEYHRPPGALHTCGGPGAFHLIVHLKAGDAVNVV
VTGGKLAHTDFDEMYSTFSGVFLYPFLSHL

| | | |
|---|---|---|
| | Down syndrome critical region | |
| 12 | protein 8 (+ isoforms) | Q96T75 |

SEQ ID NO: 43

MKEPGPNFVTVRKGLHSFKMAFVKHLLLFLSPRLECSGSITDHCSLHLPV
QEILMSQPPEQLGLQTNLGNQESSGMMKLFMPRPKVLAQYESIQFMP

SEQ ID NO: 45

MHNIMMVKLKKKKSTTNLGNQESSGMMKLFMPRPKVLAQYESIQFMP

SEQ ID NO: 46

MSQPPEQLGLQTNLGNQESSGMMKLFMPRPKVLAQYESIQFMP

SEQ ID NO: 47

MYSYWRLECSGSITDHCSLHLPVQEILMSQPPEQLGLQTNLGNQESSGMM
KLFMPRPKVLAQYESIQFMP

SEQ ID NO: 48

MKEPGPNFVTVRKGLHSFKMAFVKHLLLECSGSITDHCSLHLPVQEILMS
QPPEQLGLQTNLGNQESSGMMKLFMPRPKVLAQYESIQFMP

SEQ ID NO: 49

MKEPGPNFVTVRKGLHSFKMAFVKHLLQTLEIKKVLE

| | | |
|---|---|---|
| | Probable G-protein coupled receptor | |
| 13 | 113 [precursor] | Q8IZF5 |

SEQ ID NO: 50

MVCSAAPLLLLATTLPLLGSPVAQASQPVSETGVRPREGLQRRQWGPLIG
RDKAWNERIDRPFPACPIPLSSSFGRWPKGQTMWAQTSTLTLTEEELGQS
QAGGESGSGQLLDQENGAGESALVSVYVHLDFPDKTWPPELSRTLTLPAA
SASSSPRPLLTGLRLTTEGNVNHKGNFYCACLSGYQWNTSICLHYPPCQS
LHNHQPCGCLVFSHPEPGYCQLLPFGSPVTCLPAVPGILNLNSQLQMPGD
TLSLTLHLSQEATNLSWFLRHPGSPSPILLQPGTQVSVTSSHGQAALSVS
NMSHHWAGEYMSCFEAQGFKWNLYEVVRVPLKATDVARLPYQLSISCATS
PGFQLSCCIPSTNLAYTAAWSPGEGSKASSFNESGSQCFVLAVQRCPMAD
TTYACDLQSLGLAPLRVPISITIIQDGDITCPEDASVLTWNVTKAGHVAQ
APCPESKRGIVRRLCGADGVWGPVHSSCTDARLLALFTRTKLLQAGQGSP
AEEVPQILAQLPGQAAEASSPSDLLTLLSTMKYVAKVVAEARIQLDRRAL
KNLLIATDKVLDMDTRSLWTLAQARKPWAGSTLLLAVETLACSLCPQDHP
FAFSLPNVLLQSQLFGPTFPADYSISFPTRPPLQAQIPRHSLAPLVRNGT
EISITSLVLRDLDHLLPSNYGQGLGDSLYATPGLVLVISIMAGDRAFSQG
EVIMDFGNTDGSPHCVGWDHSLFQGRGGWSKEGCQAQVASASPTAQCLCQ
HLATFSVLMSPHTVPEEPALALLTQVGLGASILALLVCLGVYWLVWRVVV
RNKISYFRHAALLNMVFCLLAADTCFLGAPFLSPGPRSPLCLAAAFLCHF
LYLATFFWMLAQALVLAHQLLFVFHQLAKHRVLPLMVLLGYLCPLGLAGV
TLGLYLPQGQYLREGECWLDGKGGALYTFVGPVLAIIGVNGLVLAMAMLK
LLRPSLSEGPPAEKRQALLGVIKALLILTPIFGLTWGLGLATLLEEVSTV
PHYIFTILNTLQGVFILLFGCLMDRKIQEALRKRFCRAQAPSSTISLVSC
CLQILSCASKSMSEGIPWPSSEDMGTARS

| | | |
|---|---|---|
| | ANXA4 protein [fragment] | |
| 14 | (+ isoforms) | Q6LES2 |

SEQ ID NO: 52

MAMATKGGTVKAASGFNAMEDAQTLRKAMKGLGTDEDAIISVLAYRNTAQ
RQEIRTAYKSTIGRDLIDDLKSELSGNFEQVIVGMMTPTVLYDVQELRRA
MKGAGTDEGCLIEILASRTPEEIRRISQTYQQQYGRSLEDDIRSDTSFMF
QRVLVSLSAGGRDEGNYLDDALVRQDAQDLYEAGEKKWGTDEVKFLTVLC
SRNRNHLLHVFDEYKRISQKDIEQSIKSETSGSFEDALLAIVKCMRNKSA
YFAEKLYKSMKGLGTDDNTLIRVMVSRAEIDMLDIRAHFKRLYGKSLYSF
IKGDTSGDYRKVLLVLCGGDD

SEQ ID NO: 54

ATKGGTVKAASGFNAMEDAQTLRKAMKGLGTDEDAIISVLAYRNTAQRQE
IRTAYKSTIGRDLIDDLKSELSGNFEQVIVGMMTPTVLYDVQELRRAMKG
AGTDEGCLIEILASRTPEEIRRISQTYQQQYGRSLEDDIRSDTSFMFQRV
LVSLSAGGRDEGNYLDDALVRQDAQDLYEAGEKKWGTDEVKFLTVLCSRN
RNHLLHVFDEYKRISQKDIEQSIKSETSGSFEDALLAIVKCMRNKSAYFA
EKLYKSMKGLGTDDNTLIRVMVSRAEIDMLDIRAHFKRLYGKSLYSFIKG
DTSGDYRKVLLVLCGGDD

| | | |
|---|---|---|
| | Uromodulin-like 1 [precursor] | |
| 15 | (+ isoforms) | Q5DIDO |

SEQ ID NO: 56

MLRTSGLALLALVSAVGPSQASGFTEKGLSLLGYQLCSHRVTHTVQKVEA
VQTSYTSYVSCGGWIPWRRCPKMVYRTQYLVVEVPESRNVTDCCEGYEQL
GLYCVLPLNQSGQFTSRPGACPAEGPEPSTSPCSLDIDCPGLEKCCPWSG
GRYCMAPAPQAPERDPVGSWYNVTILVKMDFKELQQVDPRLLNHMRLLHS
LVTSALQPMASTVHHLHSAPGNASTTVSRLLLGLPRPLPVADVSTLLGDI
AKRVYEVISVQVQDVNECFYEELNACSGRELCANLEGSYWCVCHQEAPAT
SPRKLNLEWEDCPPVSDYVVLNVTSDSFQVSWRLNSTQNHTFHVRVYRGM
ELLRSARTQSQALAVAGLEAGVLYRVKTSYQGCGADVSTTLTIKTNAQVF
EVTIKIVNHNLTEKLLNRSSVEYQDFSRQLLHEVESSFPPVVSDLYRSGK
LRMQIVSLQAGSVVVRLKLTVQDPGFPMGISTLAPILQPLLASTVFQIDR
QGTRVQDWDECVDSAEHDCSPAAWCINLEGSYTCQCRTTRDATPSRAGRA
CEGDLVSPMGGGLSAATGVTVPGLGTGTAALGLENFTLSPSPGYPQGTPA
AGQAWTPEPSPRRGGSNVVGYDRNNTGKGVEQELQGNSIMEPPSWPSPTE
DPTGHFLWHATRSTRETLLNPTWLRNEDSGPSGSVDLPLTSTLTALKTPA
CVPVSIGRIMVSNVTSTGFHLAWEADLAMDSTFQLTLTSMWSPAVVLETW
NTSVTLSGLEPGVLHLVEIMAKACGKEGARAHLKVRTAARKLIGKVRIKN
VRYSESFRNASSQEYRDFLELFFRMVRGSLPATMCQHMDAGGVRMEVVSV
TNGSIVVEFHLLIIADVDVQEVSAAFLTAFQTVPLLEVIRGDTFIQDYDE
CERKEDDCVPGTSCRNTLGSFTCSCEGGAPDFPVEYSERPCEGDSPGNET
WATSPERPLTTAGTKAAFVQGTSPTPQGLPQRLNLTGAVRVLCEIEKVVV
AIQKRFLQQESIPESSLYLSHPSCNVSHSNGTHVLLEAGWSECGTLMQSN
MTNTVVRTTLRNDLSQEGIIHHLKILSPIYCAFQNDLLTSSGFTLEWGVY
TIIEDLHGAGNFVTENQLFIGDSPIPQNYSVSASDDVRIEVGLYRQKSNL
KVVLTECWATPSSNARDPITFSFINNSCPVPNTYTNVIENGNSNKAQFKL
RIFSFINDSIVYLHCKLRVCMESPGATCKINCNNFRLLQNSETSATHQMS
WGPLIRSEGEPPHAEAGLGAGYVVLIVVAIFVLVAGTATLLIVRYQRMNG
RYNFKIQSNNFSYQVFYE

SEQ ID NO: 58

MLRTSGLALLALVSAVGPSQASGFTEKGLSLLGYQLCSHRVTHTVQKVEA
VQTSYTSYVSCGGWIPWRRCPKMVYRTQYLVVEVPESRNVTDCCEGYEQL
GLYCVLPLNQSGQFTSRPGACPAEGPEPSTSPCSLDIDCPGLEKCCPWSG
GRYCMAPAPQAPERDPVGSWYNVTILVKMDFKELQQVDPRLLNHMRLLHS
LVTSALQPMASTVHHLHSAPGNASTTVSRLLLGLPRPLPVADVSTLLGDI
AKRVYEVISVQVQDVNECFYEELNACSGRELCANLEGSYWCVCHQEAPAT
SPRKLNLEWEDCPPVSDYVVLNVTSDSFQVSWRLNSTQNHTFHVRVYRGM
ELLRSARTQSQALAVAGLEAGVLYRVKTSYQGCGADVSTTLTIKTNAQVF
EVTIKIVNHNLTEKLLNRSSVEYQDFSRQLLHEVESSFPPVVSDLYRSGK
LRMQIVSLQAGSVVVRLKLTVQDPGFPMGISTLAPILQPLLASTVFQIDR
QGTRVQDWDECVDSAEHDCSPAAWCINLEGSYTCQCRTTRDATPSRAGRA
CEGDLVSPMGGGLSAATGVTVPGLGTGTAALGLENFTLSPSPGYPQGTPA
AGQAWTPEPSFRRGGSNVVGYDRNNTGKGVEQEVPSTAPGLGMDQGSPSQ
VNPSQGSPSQGSLRQESTSQASPSQRSTSQGSPSQVNPSQGSTSHANSSQ
GSPSQGSPSQESPSQGSTSQASPSHRNTIGVIGTTSSPKATGSTHSFPPG
ATDGPLALPGQLQGNSIMEPPSWPSPTEDPTGHFLWHATRSTRETLLNPT
WLRNEDSGPSGSVDLPLTSTLTALKTPACVPVSIGRIMVSNVTSTGFHLA
WEADLAMDSTFQLTLTSMWSPAVVLETWNTSVTLSGLEPGVLHLVEIMAK
ACGKEGARAHLKVRTAARKLIGKVRIKNVRYSESFRNASSQEYRDFLELF
FRMVRGSLPATMCQHMDAGGVRMEVVSVTNGSIVVEFHLLIIADVDVQEV
SAAFLTAFQTVPLLEVIRGDTFIQDYDECERKEDDCVPGTSCRNTLGSFT
CSCEGGAPDFPVEYSERPCEGDSPGNETWATSPERPLTTAGTKAAFVQGT
SPTPQGLPQRLNLTGAVRVLCEIEKVVVAIQKRFLQQESIPESSLYLSHP
SCNVSHSNGTHVLLEAGWSECGTLMQSNMTNTVVRTTLRNDLSQEGIIHH
LKILSPIYCAFQNDLLTSSGFTLEWGVYTIIEDLHGAGNFVTEMQLFIGD
SPIPQNYSVSASDDVRIEVGLYRQKSNLKVVLTECWATPSSNARDPITFS
FINNSCPVPNTYTNVIENGNSNKAQFKLRIFSFINDSIVYLHCKLRVCME
SPGATCKINCNNFRLLQNSETSATHQMSWGPLIRSEGEPPHAEAGLGAGY
VVLIVVAIFVLVAGTATLLIVRYQRMNGRYNFKIQSNNFSYQVFYE

SEQ ID NO: 59

MVYRTQYLVVEVPESRNVTDCCEGYEQLGLYCVLPLNQSGQFTSRPGACP
AEGPEPSTSPCSLDIDCPGLEKCCPWSGGRYCMAPAPQAPERDPVGSWYN
VTILVKMDFKELQQVDPRLLNHMRLLHSLVTSALQPMASTVHHLHSAPGN
ASTTVSRLLLGLPRPLPVADVSTLLGDIAKRVYEVISVQVQDVNECFYEE
LNACSGRELCANLEGSYWCVCHQEAPATSPRKLNLEWEDGPPVSDYVVLN
VTSDSFQVSWRLNSTQNHTFHVRVYRGMELLRSARTQSQALAVAGLEAGV
LYRVKTSYQGCGADVSTTLTIKTNAQVFEVTIKIVNHNLTEKLLNRSSVE
YQDFSRQLLHEVESSFPPVVSDLYRSGKLRMQIVSLQAGSVVVRLKLTVQ
DPGFPMGISTLAPILQPLLASTVFQIDRQGTRVQDWDECVDSAEHDCSPA
AWCINLEGSYTCQCRTTRDATPSRAGRACEGDLVSPMGGGLSAATGVTVP
GLGTGTAALGLENFTLSPSPGYPQGTPAAGQAWTPEPSPRRGGSNVVGYD
RNNTGKGVEQELQGNSIMEPPSWPSPTEDPTGHFLWHATRSTRETLLNPT
WLRNEDSGPSGSVDLPLTSTLTALKTPACVPVSIGRIMVSNVTSTGFHLA
WEADLAMDSTFQLTLTSMWSPAVVLETWNTSVTLSGLEPGVLHLVEIMAK
ACGKEGARAHLKVRTAARKLIGKVRIKNVRYSESFRNASSQEYRDFLELF
FRMVRGSLPATMCQHMDAGGVRMEVVSVTNGSIVVEFHLLIIADVDVQEV
SAAFLTAFQTVPLLEVIRGDTFIQDYDECERKEDDCVPGTSCRNTLGSFT
CSCEGGAPDFPVEYSERPCEGDSPGNETWATSPERPLTTAGTKAAFVQGT
SPTPQGLPQRLNLTGAVRVLCEIEKVVVAIQKRFLQQESIPESSLYLSHP
SCNVSHSNGTBVLLEAGWSECGTLMQSNMTNTVVRTTLRNDLSQEGIIHH
LKILSPIYCAFQNDLLTSSGFTLEWGVYTIIEDLHGAGNFVTEMQLFIGD
SPIPQNYSVSASDDVRIEVGLYRQKSNLKVVLTECWATPSSNARDPITFS
FINNSCPVPNTYTNVIENGNSNKAQFKLRIFSFINDSIVYLHCKLRVCME
SPGATCKINCNNFRLLQNSETSATHQMSWGPLIRSEGEPPHAEAGLGAGY
VVLIVVAIFVLVAGTATLLIVRYQRMNGRYNFKIQSNNFSYQVFYE

SEQ ID NO: 60

MVYRTQYLVVEVPESRNVTDCCEGYEQLGLYCVLPLNQSGQFTSRPGACP
AEGPEPSTSPCSLDIDCPGLEKCCPWSGGRYCMAPAPQAPERDPVGSWYN
VTILVKMDFKELQQVDPRLLNHMRLLHSLVTSALQPMASTVHHLHSAPGN
ASTTVSRLLLGLPRPLPVADVSTLLGDIAKRVYEVISVQVQDVNECFYEE
LNACSGRELCANLEGSYWCVCHQEAPATSPRKLNLEWEDCPPVSDYVVLN
VTSDSFQVSWRLNSTQNHTFHVRVYRGMELLRSARTQSQALAVAGLEAGV
LYRVKTSYQGCGADVSTTLTIKTNAQVFEVTIKIVNHNLTEKLLNRSSVE
YQDFSRQLLHEVESSFPPVVSDLYRSGKLRMQIVSLQAGSVVVRLKLTVQ
DPCFPMCISTLAPILQPLLASTVFQIDRQGTRVQDWDECVDSAEHDCSPA
AWCINLEGSYTCQCRTTRDATPSRAGRACEGDLVSPMGGGLSAATGVTVP
GLGTGTAALGLENFTLSPSPGYPQGTPAAGQAWTPEPSPRRGGSNVVGYD
RNNTGKGVEQEVPSTAPGLGMDQGSPSQVNPSQGSPSQGSLRQESTSQAS
PSQRSTSQGSPSQVNPSQGSTSHANSSQGSPSQGSPSQESPSQGSTSQAS
PSHRNTIGVIGTTSSPKATGSTHSFPPGATDGPLALPGQLQGNSIMEPPS
WPSPTEDPTGHFLWHATRSTRETLLNPTWLRNEDSGPSGSVDLPLTSTLT
ALKTPACVPVSIGRIMVSNVTSTGFHLAWEADLAMDSTFQLTLTSMWSPA
VVLETWNTSVTLSGLEPGVLHLVEIMAKACGKEGARAHLKVRTAARKLIG
KVRIKNVRYSESFRNASSQEYRDFLELFFRMVRGSLPATMCQHMDAGGVR
MEVVSVTNGSIVVEFHLLIIADVDVQEVSAAFLTAFQTVPLLEVIRGDTF
IQDYDECERKEDDCVPGTSCRNTLGSFTCSCEGGAPDFPVEYSERPCEGD
SPGNETWATSPERPLTTAGTKAAFVQGTSPTPQGLPQRLNLTGAVRVLCE
IEKVVVAIQKRFLQQESIPESSLYLSHPSCNVSHSNGTHVLLEAGWSECG
TLMQSNMTNTVVRTTLRNDLSQEGIIHHLKILSPIYCAFQNDLLTSSGFT
LEWGVYTIIEDLHGAGNFVTEMQLFIGDSPIPQNYSVSASDDVRIEVGLY
TQKSNLKVVLTECWATPSSNARDPITFSFINNSCPVPNTYTNVIENGNSN
KAQFKLRIFSFINDSIVYLHCKLRVCMESPGATCKINCNNFRLLQNSETS
ATHQMSWGPLIRSEGEPPHAEAGLGAGYVVLIVVAIFVLVAGTATLLIVR
YQRMNGRYNFKIQSNNFSYQVFYE

| | | |
|---|---|---|
| | Scavenger receptor class F member 2 | |
| 16 | [precursor] | Q96GP6 |

SEQ ID NO: 61

MEGAGPRGAGPARRRGAGGPPSPLLPSLLLLLLLWMLPDTVAPQELNPRG
RNVCRAPGSQVPTCCAGWRQQGDECGIAVCEGNSTCSENEVCVRPGECRC
RHGYFGANCDTKCPRQFWGPDCKELCSCHPHGQCEDVTGQCTCHARRWGA
RCEHACQCQHGTCHPRSGACRCEPGWWGAQCASACYCSATSRCDPQTGAC
LCHAGWWGRSCNNQCACNSSPCEQQSGRCQCRERTFGARCDRYCQCFRGR
CHPVDGTCACEPGYRGKYCREPCPAGFYGLGCRRRCGQCKGQQPCTVAEG
RCLTCEPGWNGTKCDQPCATGFYGEGCSHRCPPCRDGHACNHVTGKCTRC
NAGWIGQRCETKCSNGTYGEDCAFVCADCGSGHCDFQSGRCLCSPGVHGP
HCNVTCPPGLHGADCAQACSCHEDTCDPVTGACHLETNQRKGVMGAGALL
VLLVCLLLSLLGCCCACRGKDPTRRELSLGRKKAPHRLCGRFSRISMKLP
RIPLRRQKLPKVVVAHHDLDNTLNCSFLEPPSGLEQPSPSWSSRASFSSF
DTTDEGPVYCVPHEEAPAESRDPEVPTVPAEAPAPSPVPLTTPASAEEAI
PLPASSDSERSASSVEGPGGALYARVARREARPARARGEIGGLSLSPSPE
RRKPPPPDPATKPKVSWIHGKHSAAAAGRAPSPPPPGSEAAPSPSKRKRT
PSDKSAHTVEHGSPRTRDPTPRPPGLPEEATALAAPSPPRARARGRGPGL
LEPTDAGGPPRSAPEAASMLAAELRGKTRSLGRAEVALGAQGPREKPAPP
QKAKRSVPPASPARAPPATETPGPEKAATDLPAPETPRKKTPIQKPPRKK
SREAAGELGRAGAPTL

| | | |
|---|---|---|
| | Sushi domain-containing protein 2 | |
| 17 | precursor | Q9UGT4 |

SEQ ID NO: 63

MKPALLPWALLLLATALGPGPGPTADAQESCSMRCGALDGPCSCHPTCSG
LGTCCLDFRDFCLEILPYSGSMMGGKDFVVRHFKMSSPTDASVICRFKDS
IQTLGHVDSSGQVHCVSPLLYESGRIPFTVSLDNGHSFPRAGTWLAVHPN
KVSMMEKSELVNETRWQYYGTANTSGNLSLTWHVKSLPTQTITIELWGYE
ETGMPYSQEWTAKWSYLYPLATHYPNSGSFTFTPKPAPPSYQRWRVGALR
TIDSKNYAGQKDVQALWTNDHALAWHLSODFREDPVAWARTQCQAWEELE
DQLPNFLEELPDCPCTLTQARADSGRFFTDYGCDMEQGSVCTYHPGAVHC
VRSVQASLRYGSGQQCCYTADGTQLLTADSSGGSTPDRGHDWGAPPFRTP
PRVPSMSHWLYDVLSFYYCCLWAPDCPRYMQRRPSNDCRNYRPPRLASAF
GDPHFVTFDGTNFTFNGRGEYVLLEAALTDLRVQARAQPGTMSNGTETRG
TGLTAVAVQEGNSDVVEVRLANRTGGLEVLLNQEVLSFTEQSWMDLKGMF
LSVAAGDRVSIMLASGAGLEVSVQGPFLSVSVLLPEKFLTHTHGLLGTLN
NDPTDDFTLHSGRVLPPGTSPQELFLFGANWTVHNASSLLTYDSWFLVHN
FLYQPKHDPTFEPLFPSETTLNPSLAQEAAKLCGDDHFCNFDVAATGSLS
TGTATRVAHQLHQRRMQSLQPVVSCGWLAPPPNGQKEGNRYLAGSTIYFH
CDNGYSLAGAETSTCQADGTWSSPTPKCQPGRSYAVLLGIIFGGLAVVAA
VALVYVLLRRRKGNTHVWGAQP

| | | |
|---|---|---|
| | Tumor protein, translationally | |
| 18 | controlled 1 | Q5W0H4 |

SEQ ID NO: 65

MIIYRDLISHDEMFSDIYKIREIADGLCLEVEGKMVSRTEGNIDDSLIGG
NASAEGPEGEGTESTVITGVDIVMNHHLQETSFTKEAYKKYIKDYMKSIK
GKLEEQRPERVKPFMTGAAEQIKHILANFKNYQFFIGENMNPDGMVALLD
YREDGVTPYMIFFKDGLEMEKCVSTRKWVKINNVKKTF

| | | |
|---|---|---|
| 19 | Putative G-protein coupled receptor | Q8TDUO |

SEQ ID NO: 67

MVLCCRGSLLWMVLCCGWFSVIEGLCCGGSLLWMVLCCGWVSVVGGSLLW
RVLCCGGFSVVEGSLLWRVSVGDGSLLWRLSVVEVLCCGGSLLWMVLCCG
GFCVVEGSLLWRVSVVDGSLSWRLSVVEAPCRGGSLSWRVSVVGGSLLWR
VSVVDGSLLWRVSVVEGLCCGGFSVVEGLCCGGFSVVEGCLLWRVLCCGA
SLLWRVSVVEGSLLWRVCCGGFSVVESLCCGWFSVVEGSLLWRVLCCGWF
SVMEGSLLWRVLSCGWFSVVDGSLLWRVSVVEGLCCGESVVDGSLLWRVS
VVEGLCCGWFFVVGGSLLWMVLCCGWFSVVNGSLLWHGLCCGGFSVVDGS
LLWMGLCCGGSLLWMVLCFGWVSVVEVLCCGSSLLYLLQICMIRCLGAST
H

| | Hypothetical protein DKFZp686KQ275 | Q7Z3AI |
|---|---|---|
| 20 | [fragment] | |

SEQ ID NO: 69

DRSRWRGRAGQGFGLRRREMAAGGRMEDGSLDITQSIEDDPLLDAQLLPH
HSLQAHFRPRFHPLPTVIIVNLLWFIHLVFVVLAFLTGVLCSYPNPNEDK
CPGNYTNPLKVQAVIILGKVILWILHLLLECYIQYHHSKIRNRGYNLIYR
STRHLKRLALMIQSSGNTVLLLILCMQHSFPKPGRLYLDLILAILALELI
CSLICLLIYTVKIRRFNKAKPEPDILEEEKIYAYPSNITSETGFRTISSL
EEIVEKQGDTIEYLKRHNALLSKRLLALTSSDLGCQPSRT

| | | |
|---|---|---|
| 21 | Transmembrane protein TMEM55A | Q8N4L2 |

SEQ ID NO: 71

MAADGVDERSPLLSASHSGNVTPTAPPYLQESSPRAELPPPYTAIASPDA
SGIPVINCRVCQSLINLDGKLHQHVVKCTVCNEATPIKNPPTGKKYVRCP
CNCLLICKDTSRRIGCPRPNCRRIINLGPVMLISEEQPAQPALPIQPEGT
RVVCGHCGNTFLWMELRFNTLAKCPHCKKISSVGSALPERRRCCAYITIG
MICIFIGVGLTVGTPDFARRFRATYVSWAIAYLLGLICLIRACYWGAIRV
SYPEHSFA

| | | |
|---|---|---|
| 22 | Hypothetical protein Q8WYY4 | Q8WYY4 |

SEQ ID NO: 73

MVLTAMVGKIHRKRLTYTNAGRIKKLTQTNVADVVKLHKGDMHGCAWWLV
PVILALGGAGAGGSLEARSSRPAWPTWRSPVSTKNTRVGQAWWSMPVISA
TWETEVGGSLGPRRQRVQ

| | | |
|---|---|---|
| | Family with sequence similarity | |
| 23 | 116, member A | Q8IWF6 |

SEQ ID NO: 75

MALRGPAGLGPGSRRPLDEAVAGAEGREAPALVAAGGAPEDDEEDDGRGR
GLLRWDSFSAWLHCVCVVGFDLELGQAVEVIYPQHSKLTDREKTNICYLS
FPDSNSGCLGDTQFCFRFRQSSGRRVSLHCLLDQFDKDLPVYLKKDPAYF
YGYVYFRQVRDKTLKRGYFQKSLVLISKLPYIHFFHTVLKQIAPEYFEKN
EPYLEAACNDVDRWPAPVPGKTLHLPIMGVVMKVRIPTCHDKPGTTQIVQ
LTQQVDTNISVILPTVHEVDIFRCFCPVFLHSQMLWELVLLGEPLVVMAP
SPSESSETVLALVNCISPLKYFSDFRPYFTIHDSEFKEYTTRTQAPPSVI
LGVTNPFFAKTLQHWPHIIRIGDLKPTGEIPKQVKVKKLKNLKTLKSKPG
VYTSYKPYLNRDEEIIKQLQKGVQQKRPSEAQSVILRRYFLELTQSFIIP
LERYVASLMPLQKSISPWKSPPQLRQFLPEEFMKTLEKTGPQLTSRIKGD
WIGLYRHFLKSPNFDGWFKTRRKEMTQKLEALHLEALCEEDLLLWIQKHT
EVETVDLVLKLKNKLLQADREHLPVKPDTMEKLRTHIDAIILALPEDLQG
ILLKTGMT

| | | |
|---|---|---|
| 24 | UPF0240 protein C6orf66 | Q9P032 |

SEQ ID NO: 77

MGALVIRGIRNFNLENRAEREISKMKPSVAPRHPSTNSLLREQISLYPEV
KGEIARKDEKLLSFLKDVYVDSKDPVSSLQVKAAETCQEPKEFRLPKDHH
FDMINIKSIPKGKISIVEALTLLNNHKLFPETWTAEKIMQEYQLEQKDVN
SLLKYFVTFEVEIFPPEDKKAIRSK

| | | |
|---|---|---|
| | CDNA FLJ45811 fis, clone | |
| 25 | NT2RP7014778 | Q6ZS59 |

SEQ ID NO: 79

MVFYCMHLKYYSEKAPKGPQGKNNYDPIGLGTQYPKVWHFGMLSALNQRR
LEGLRRKVSLNLSYSPVSDFFFPYKGSHRNQNSSSVYYHEIIPFCPVTFL
QGCPFFMECKHKNRQFWLGGVARACNPSTLGG

| | | |
|---|---|---|
| 26 | Hypothetical protein DKFZp779O1248 | Q6AHZ8 |

SEQ ID NO: 81

MAGAEWKSLEECLEKHLPLPDLQEVKRVLYGKELRKLDLPREAFEAASRE
DFELQGYAFEAAEEQLRRPRIVHVGLVQNRIPLPANAPVAEQVSALHRRI
KAIVAVAAMCGVNIICFQEAWILRPHHQEPRPPCCYAPSCCLIPSVFPHR
SESIRSSPSLYHHLVQAPGSSPLAFVIPSSWSPVPS

| | | |
|---|---|---|
| 27 | Beta-ureidopropionase | Q9UBR1 |

SEQ ID NO: 83

MAGAEWKSLEECLEKHLPLPDLQEVKRVLYGKELRKLDLPREAFEAASRE
DFELQGYAFEAAEEQLRRPRIVHVGLVQNRIPLPANAPVAEQVSALHRRI
KAIVEVAAMCGVNIICFQEAWTMPFAFCTPEKLPWTEFAESAEDGPTTRF
CQKLAKNHDMVVVSPILERDSEHGDVLWNTAVVISNSGAVLGKTRKNHIP
RVGDFNESTYYMEGNLGHPVFQTQFGRIAVNICYGRHHPLNWLMYSINGA
EIIFNPSATIGALSESLWPIEARNAAIANHCFTCAINRVGTEHFPNEFTS
GDGKKAHQDFGYGYGSSYVAAPDSSRTPGLSRSEDGLLVAKLDLNLCQQV
NDVWNFKMTGRYEMYARELAEAVKSNYSPTIVKE

| | | |
|---|---|---|
| | Hypothetical protein DKFZp434F1919 | |
| 28 | (+ isoforms) | Q9GZU6 |

SEQ ID NO: 85

MNSRQAWRLFLSQGRGDRWVSRPRGHFSPALRREFFTTTTKEGYDRRFVD
ITPLEQRKLTFDTHALVQDLETHGFDKTQAETIVSALTALSNVSLDTIYK
EMVTQAQQEITVQQLMAHLDAIRKDMVILEKSEFANLRAENEKMKIELDQ
VKQQLMHETSRIRADNKLDINLERSRVTDMFTDQEKQLMETTTEFTKKDT
QTKSIISETSNKIDAEIASLKTLMESNKLETIRYLAASVFTCLAIALGFY
RFWK

SEQ ID NO: 87

MNSRQAWRLLLSQGRGDRWVSRPRGHFSPALRREFFTTTTKEGYDRRFVD
ITPLEQRKLTFDTHALVQDLETHGFDKTQAETIVSALTALSNVSLDTIYK
EMVTQAQQEITVQQLMAHLDAIRKDMVILEKSEFANLRAENEKMKIELDQ
VKQQLMHETSRIRADNKLDINLERSRVTDMFTDQEKQLMETTTEFTKKDT
QTKSIISETSNKIDAEIASLKTLMESNKLETIRYLAASVFTCLAIALGFY
RFWK

| | | |
|---|---|---|
| | Cysteine-rich with EGE-like domain | |
| | protein 2 [precursor] | |
| 29 | (+ isoforms) | Q6UXHI |

SEQ ID NO: 89

MRLPRRAALGLLPLLLLLPPAPEAAKKPTPCHRCRGLVDKFNQGMVDTAK
KNFGGGNTAWEEKTLSKYESSEIRLLEILEGLCESSDFECNQMLEAQEEH
LEAWWLQLKSEYPDLFEWFCVKTLKVCCSPGTYGPDCLACQGGSQRPCSG
NGHCSGDGSRQGDGSCRCHMGYQGPLCTDCMDGYFSSLRNETHSICTACD
ESCKTCSGLTNRDCGECEVGWVLDEGACVDVDECAAEPPPCSAAQFCKNA
NGSYTCEECDSSCVGCTGEGPGNCKECISGYAREHGQCADVDECSLAEKT
CVRKNENCYNTPGSYVCVCPDGFEETEDACVPPAEAEATEGESPTQLPSR
EDL

SEQ ID NO: 91

MRLPRRAALGLLPLLLLLPPAPEAAKKPTPCHRCRGLVDKFNQGMVDTAK
KNFGGGNTAWEEKTLSKYESSEIRLLEILEGLCESSDFECNQMLEAQEEH
LEAWWLQLKSEYPDLFEWFCVKTLKVCCSPGTYGPDCLACQGGSQRPCSG
NGHCSGDGSRQGDGSCRCHMGYQGPLCTDCMDGYFSSLRNETHSICTACD
ESCKTCSGLTNRDCGECEVGWVLDEGACVDVDECAAEPPPCSAAQFCRNA
NGSYTCEDVDECSLAEKTCVRKNENCYNTPGSYVCVCPDGFEETEDACVP
PAEAEATEGESPTQLPSREDL

SEQ ID NO: 92

MRLPRRAALGLLPLLLLLPPAPEAAKKPTPCHRCRGLVDKFNQGMVDTAK
KNFGGGNTAWEEKTLSKYESSEIRLLEILEGLCESSDFECNQMLEAQEEH
LEAWWLQLKSEYPDLFEWFCVKTLKVCCSPGTYGPDCLACQGGSQRPCSG
NGHCSGDGSRQGDGSCRCHMGYQGPLCTDCMDGYFSSLRNETHSICTACD
ESCKTCSGLTNRDCGECEVGWVLDEGACVDVDECAAEPPPCSAAQFCKNA
NGSYTCEGGPGGRVCTPGPACFRCCLCQHSFMAS

SEQ ID NO: 93

MRLPRRAALGLLPLLLLLPPAPEAAKKPTPCHRCRGLVDKFNQGMVDTAK
KNFGGGNTAWEEKTLSKYESSEIRLLEILEGLCESSDEECNQMLEAQEEH
LEAWWLQLKSEYPDLFEWFCVKTLKVCCSPGTYGPDCLACQGGSQRPCSG
NGHCSGDGSRQGDGSCRCHMGYQGPLCTDCMDGYFSSLRNETHSICTACD
ESCKTCSGLTNRDCGECEVGWVLDEGACVECDSSCVGCTGEGPGNCKECI
SCYAREHGQCADVDECSLAEKTCVRKNENCYNTPGSYVCVCPDGFEETED
ACVPPAEAEATEGESPTQLPSREDL

SEQ ID NO: 94

MRLPRRAALGLLPLLLLLPPAPEAAKKPTPCHRCRGLVDKFNQGMVDTAK
KNFGGGNTAWEEKTLSKYESSEIRLLEILEGLCESSDFECNQMLEAQEEH
LEAWWLQLKSEYPDLFEWFCVKTLKVCCSPGTYCPDCLACQGGSQRPCSG
NGHCSGDGSRQGDGSCRCHMGYQGPLCTDCMDGYFSSLRNETHSICTVRT
GLSDSYPPCCLSLGCWRGVGHAWIRGRNTHTQPGYSSRVWIAAFSPACDE
SCKTCSGLTNRDCGECEVGWVLDEGACVDVDECAAEPPPCSAAQFCKNAN
GSYTCEECDSSCVGCTGEGPGNCKECISGYAREHGQCADVDECSLAEKTC
VRKNENCYNTPGSYVCVCPDGFEETEDACVPPAEAEATEGESPTQLPSRE
DL

SEQ ID NO: 95

MRLPRRAALGLLPLLLLLPPAPEAAKKPTPCHRCRGLVDKFNQGMVDTAK
KNFGGGNTAWEEKTLSKYESSEIRLLEILEGLCESSDFECNQMLEAQEEH
LEAWWLQLKSEYPDLFEWFCVKTLKVCCSPGTYGPDCLACQCGSQRPCSG
NGHCSGDGSRQCDGSCRCHMCYQGPLCTDCMDGYFSSLRNETHSICTACD
ESCKTCSGLTNRDCGECEVGWVLDEGACVDVDECAAEPPPCSAAQFCKNA
NGSYTCEECDSSCVGCTGEGPGNCKECISGYAREHGQCADVDECSLAEKT
CVRKNENCYNTPGSYVCVCPDGFEETEDACVPPAEAGEWHGCPPHRLPSP
GPQGLHVDWLLGLKSTQMVALRW

| | | |
|---|---|---|
| | UPF0378 family protein KIAA0100 | |
| 30 | [precursor] | Q5H9T4 |

SEQ ID NO: 96

MPLFFSALLVLLLVALSALFLGRWLVVRLATKWCQRKLQAELKIGSFRFF
WIQNVSLKFQQHQQTVEIDNLWISSKPLSHDLPHYVALCFGEVRIRTDLQ
KVSDLSAPFSQSAGVDQKELSFSPSLLKIFCQLFSIHVDAINIMVLKVDT
SESLWHIQISRSRFLLDSDGKRLICEVSLCKINSKVLKSGQLEDTCLVEL
SLALDLCLKVGISSRHLTAITVDVWTLHAELHEGLFQSQLLCQGPSLASK
PVPCSEVTENLVEPTLPGLFLLQQLPDQVKVKMENTSVVLSMNSQKRHLT
WTLKLLQFLYHRDEDQLPLRSFTANSDMAQMSTELLLEDGLLLSQSRQRI
VCLNSLKASVQVTTIDLSASLVLNTCIIHYRHQEFSHWLHLLALETQGSS
SPVLKQRKKRTFPQILAPIIFSTSISNVNISIQLGDTPPFALGFNSISLD
YQHLRPQSIHQRGVLTVDHLCWRVGSDSHIQRAPHPPNMHVWGEALVLDS
FTLQGSYNQPLVLSSTQSDTLFLDCTIRGLQVEASDTCAQCLSRILSLMG
PQSGKSAVSRHSSFGESVSLLWKVDLKVEDMNLFTLSALVGASEVRLDTL
AILGSAETSTVGIQGLVLALVKSVTEKMQPCCKAPDIPTPVLSLSMLSIT
YHSSIRSLEVQCGAGLTLLWSPPDHMYLYQHVLATLQCRDLLRATVFPET
VPSLALETSGTTSELEGRAPEPLPPKRLLNLTLEVSTAKLTAFVAEDKFI
TLAAESVSLSRHGGSLQAYCPELAAGFDGNSIFNFKEVEVQLLPELEFPY
QYDFSRTLDEAVGVQKWLKGLHQGTRAWASPSPVPLPPDLLLKVEHFSWV
FLDDVFEVKLHDNYELMKDESKESAKRLQLLDAKVAALRKQHGELLPARK
IEELYASLERKNIEIYIQRSRRLYGNTPMRRALLTWSLAGLELVALADAS
FHGPEHVVEQVQELDPGSPFPPEGLDLVIQWCRMLKCNVKSFLVRIRDYP
RYLFEIRDWRLMGRLVGTEQSGQPCSRRRQILHLGLPWGNVAVERNMPPL
KFYHDFHSEIFQYTVVWGPCWDPAWTLIGQCVDLLTKPSADPSPPLPWWD
KSRLLFHGDWHMDIEQANLHQLATEDPYNTTENMHWEWSHLSFHWKPGQF
VFKGDLDINVRTASKYDDCCFLHLPDLCMTLDLQWLCHGNPHDHHSVTLR
APEFLPEVPLGQLHDSYRAFRSENLNLSIKMDLARHSGTISQPRILLYSS
TLRWMQNFWATWTSVTRPICRGKLFNNLKPSKKKLGQHYKQLSYTALFPQ
LQVHYWASFAQQRGIQIECSQGHVFTRGTQRLIPQAGTVMRRLISDWSVT
QMVSDLSQVTVHLMASPTEENADHCLDPLVTKTHLLSLSSLTYQRHSNRT
AEEELSARDGFPTFHTHQLHLVDLRISWTTTNRDIAFGLYDGYKKAAVLK
RNLSTEALKGLKIDPQMPAKKPKRGVPTSASAPPRVNTPSFSGQPDKGSS
GGAYMLQKLIEETDRLVVFTEEESGMSDQLCGIAACQTDDIYNRNCLIEL
VNCQMVLRGAETEGCVIVSAAKAQLLQCQHHPAWYGDTLKQKTSWTCLLD
GMQYFATTESSPTEQDGRQLWLEVKNIEEHRQRSLDSVQELMESGQAVGG
MVTTTTDWNQPAEAQQAQQVQRIISRCNCRMYYISYSHDIDPELATQIKP
PEVLENQEKEDKKLLQEGAVDTFTLIHHELEISNPAQYAMILDIVNNLLL
HVEPKRKEHSEKKQRVRFQLEISSNPEEQRSSILHLQEAVRQHVAQIRQL
EKQMYSTMKSLQDDSKNENLLDLNQKLQLQLNQEKANLQLESEELNILIR
CFKDFQLQRANKMELRKQQEDVSVVRRTEFYFAQARWRLTEEDGQLGIAE
LELQRFLYSKVNKSDDTAEHLLELGWFTMNNLLPNAVYKVVLRPQSSCQS
GRQLALRLFSKVRPPVGGISVKEHFEVNVVPLTIQLTHQFFHRMMGFFFP
GRSVEDDEVGDEEDKSKLVTTGIPVVKPRQLIATDDAVPLGPGKGVAQGL
TRSSGVRRSFRKSPEHPVDDIDKMKERAAMNNSFIYIKIPQVPLCVSYKG
EKNSVDWGDLNLVLPCLEYHNNTWTWLDFANAVKRDSRKALVAQVIKEKL
RLKSATGSEVRGKLETKSDLNMQQQEEEEKARLLIGLSVGDKNPGKKSIF
GRRK

| | | |
|---|---|---|
| | Potassium voltage-gated channel | |
| | subfamily H member 1 | |
| 31 | (+isoforms) | O95259 |

SEQ ID NO: 98

MTMAGGRRGLVAPQNTFLENIVRRSNDTNFVLGNAQIVDWPIVYSNDGFC
KLSGYHRAEVMQKSSTCSFMYGELTDKDTIEKVRQTFENYEMNSFEILMY
KKNRTPVWFFVKIAPIRNEQDKVVLFLCTFSDITAFKQPIEDDSCKGWGK
FARLTRALTSSRGVLQQLAPSVQKGENVHKHSRLAEVLQLGSDILPQYKQ
EAPKTPPHIILHYCVFKTTWDWIILILTFYTAILVPYNVSFKTRQNNVAW
LVVSDIVDVIFLVDIVLNFHTTFVGPAGEVISDPKLIRMNYLKTWFVIDL
LSCLPYDVINAFENVDEVSAFMGDPGKIGFADQIPPPLEGRESQGISSLF
SSLKVVRLLRLGRVARKLDHYIEYGAAVLVLLVCVFGLAAHWMACIWYSI
GDYEIFDEDTKTIRNNSWLYQLAMDIGTPYQFNGSGSGKWEGGPSKNSVY
ISSLYFTMTSLTSVGFGNIAPSTDIEKIFAVAIMMIGSLLYATIFGNVTT
IFQQMYANTNRYHEMLNSVRDFLFLYQVPKGLSERVMDYIVSTWSMSRGI
DTEKVLQICPKDMRADICVHLNRKVFKEHPAFRLASDGCLRALAMEFQTV
HCAPGDLIYHAGESVDSLCFVVSGSLEVIQDDEVVAILGKGDVFGDVFWK
EATLAQSCANVRALTYCDLHVIKRDALQKVLEFYTAFSHSFSRNLILTYN
LRKRIVFRKISDVKREEEERMKRKNEAPLILPPDHPVRRLFQRFRQQKEA
RLAAERGGRDLDDLDVEKGNVLTEHASANHSLVKASVVTVRESPATPVSF
QAASTSGVPDHAKLQAPGSECLGPKGGGGDCAKRKSWARFKDACGKSEDW
NKVSKAESMETLPERTKASGEATLKKTDSCDSGITKSDLRLDNVGEARSP
QDRSPILAEVKHSFYPIPEQTLQATVLEVRHELKEDIKALNAKMTNIEKQ
LSEILRILTSRRSSQSPQELFEISRPQSPESERDIFGAS

SEQ ID NO: 100

MTMAGGRRGLVAPQNTFLENIVRRSNDTNFVLGNAQIVDWPIVYSNDGFC
KLSGYHPAEVMQKSSTCSFMYGELTDKDTIEKVRQTFENYEMNSFEILMY
KKNRTPVWFFVKIAPIRNEQDKVVLFLCTFSDITAFKQPIEDDSCKGWGK
FARLTRALTSSRGVLQQLAPSVQKGENVHKHSRLAEVLQLGSDILPQYKQ
EAPKTPPHIILHYCVFKTTWDWIILILTFYTAILVPYNVSFKTRQNNVAW
LVVDSIVDVIFLVDIVLNFHTTFVGPAGEVISDPKLIRMNYLKTWFVIDL
LSCLPYDVINAFENVDEGISSLFSSLKVVRLLRLGRVARKLDHYIEYGAA
VLVLLVCVFGLAAHWMACIWYSIGDYEIFDEDTKTIRNNSWLYQLAMDIG
TPYQFNGSGSGKWEGGPSKNSVYISSLYFTMTSLTSVGFGNIAPSTDIEK
IFAVAIMMIGSLLYATIFGNVTTIFQQMYANTNRYHEMLNSVRDFLKLYQ
VPKGLSERVMDYIVSTWSMSRGIDTEKVLQICPKDMRADICVHLNRKVFK
EHPAFRLASDGCLRALAMEFQTVHCAPGDLIYHAGESVDSLCFVVSGSLE
VIQDDEVVAILGKGDVFGDVFWKEATLAQSCANVRALTYCDLHVIKRDAL
QKVLEFYTAFSHSFSRNLILTYNLRKRIVFRKISDVKREEEERMKRKNEA
PLILPPDHPVRRLFQRFRQQKEARLAAERGGRDLDDLDVEKGNVLTEHAS
ANHSLVKASVVTVRESPATPVSFQAASTSGVPDHAKLQAPGSECLGPKGG
GGDCAKRKSWARFKDACGKSEDWNKVSKAESMETLPERTKASGEATLKKT
DSCDSGITKSDLRLDNVGEARSPQDRSPILAEVKHSFYPIPEQTLQATVL
EVRHELKEDIKALNAKMTNIEKQLSEILRILTSRRSSQSPQELFEISRPQ
SPESERDIFGAS

1-95. (canceled)

96. A protein designated Peroxidasin homolog [fragment] having an amino acid sequence as indicated in SEQ ID NO: 33, a fragment or a functional derivative thereof.

97. The protein, fragment or functional derivative according to claim 96 having at least 70, preferably 80, more preferably 90, most preferably at least 95% amino acid sequence identity to the protein of SEQ ID NO: 33.

98. A polynucleotide encoding the protein of SEQ ID NO: 33, a fragment or a functional derivative thereof having the ability to hybridize to the nucleic acid sequence of SEQ ID NO: 34 under stringent conditions.

99. A vector comprising polynucleotides encoding proteins, fragments and functional derivatives thereof according to claim 96.

100. A host cell comprising a recombinant protein, nucleic acid and/or vector according to claim 96.

101. A ligand, preferably an antibody, fragment or functional derivative thereof having specific binding affinity to a protein according to claim 96.

102. The antibody, fragment or functional derivative thereof according to claim 101, wherein the antibody, fragment or functional derivative thereof is selected from the group consisting of polyclonal antibodies, monoclonal antibodies, chimeric antibodies, humanized antibodies, CDR-grafted antibodies, Fv-fragments, Fab-fragments, Fab₂-fragments and antibody-like binding proteins.

103. A fusion protein comprising a ligand, preferably an antibody, fragment or functional derivative thereof according to claim 102.

104. The fusion protein according to claim 103, comprising a component having anti-tumor activity or having diagnostic activity, i.e. allowing for selective identification in vivo and/or ex vivo.

105. Use of a fusion protein according to claim 104 for preparing a medicament for the treatment of cancer, preferably kidney cancer, in a mammal.

106. Use of a fusion protein according to claim 105 for preparing a diagnostic composition for the identification of tumors, preferably kidney tumors, in a mammal.

107. A pharmaceutical composition, comprising a ligand, preferably an antibody, fragment or derivative thereof, according to a ligand, preferably an antibody, fragment or functional derivative thereof having specific binding affinity to a protein designated Peroxidasin homolog [fragment] having an amino acid sequence as indicated in SEQ ID NO: 33, a fragment or a functional derivative thereof, and/or a fusion protein according to claim 103 and a pharmaceutically acceptable carrier or diluent.

108. A diagnostic composition, comprising a ligand, preferably an antibody, fragment or derivative thereof, according to a ligand, preferably an antibody, fragment or functional derivative thereof having specific binding affinity to a protein designated Peroxidasin homolog [fragment] having an amino acid sequence as indicated in SEQ ID NO: 33, a fragment or a functional derivative thereof, and/or a fusion protein according to claim 104.

109. A method for identifying tumors in mammalian tissue, preferably vascular kidney tissue, comprising:
(i) contacting a ligand, preferably an antibody, fragment or functional derivative thereof and/or a fusion protein comprising a ligand, preferably an antibody, fragment or functional derivative thereof, having specific binding affinity to the protein according to claim 1 (Peroxidasin homolog [fragment]) with mammalian tissue of interest, under conditions that allow for the specific binding of said ligand and/or fusion protein to said protein of claim 1 and
(ii) identifying specifically bound ligand and/or fusion protein, wherein the steps (i) and (ii) are performed ex vivo.

110. A method for identifying neovascular structures in mammalian tissue, preferably in mature mammalian tissue, more preferably in mature human tissue, most preferably in kidney tissue, wherein said neovascular structures are identified by the detection of the protein according to claim 96(Peroxidasin homolog [fragment]) in said tissue in vitro.

111. The method of claim 109, wherein step (i) is performed in vivo and the method is a method of imaging tumors.

112. The method of claim 110, wherein said neovascular structures are identified in vivo.

113. A method for identifying a disease or condition in a mammal selected from the group consisting of tumors, preferably human kidney tumors, macular degeneration, arthritis and/or atherosclerosis, wherein said disease or condition is identified by the detection of the protein according to claim 96 (Peroxidasin homolog [fragment]) within and/or in the close proximity of mammalian tissue of interest.

114. A method for targeting and/or imaging neovascular structures in mammalian tissue, preferably mature mammalian tissue, more preferably kidney tissue, wherein said neovascular structures are targeted and/or imaged by a ligand having specific binding affinity to the protein according to claim 96 (Peroxidasin homolog [fragment]) in said neovascular structure.

115. A method for targeting and/or imaging a tissue affected by a disease or condition in a mammal selected from the group consisting of tumors, preferably kidney tumors, macular degeneration, arthritis and/or atherosclerosis, wherein said disease or condition is targeted and/or imaged by a ligand having a specific binding affinity to the protein according to claim 96 (Peroxidasin homolog [fragment]) within and/or in the close proximity of the mammalian tissue of interest.
